SEQUENCE LISTING (45 sequences)

SEQ ID NO: 1 - protein, 8 aa, Artificial Sequence
  Preferred Intervening Spacer Peptide (Linker 1)
GGGSGGGG

SEQ ID NO: 2 - protein, 6 aa, Artificial Sequence
  Preferred Cysteine-Containing Spacer Peptide (Linker 2)
GGCGGG

SEQ ID NO: 3 - protein, 5 aa, Artificial Sequence
  Alternative Spacer Peptide (Linker 2)
ASTKG

SEQ ID NO: 4 - protein, 28 aa, Artificial Sequence
  “E-coil” Heterodimer-Promoting Domain
EVAALEKEVAALEKEVAALEKEVAALEK

SEQ ID NO: 5 - protein, 28 aa, Artificial Sequence
  “K-coil” Heterodimer-Promoting Domain
KVAALKEKVAALKEKVAALKEKVAALKE

SEQ ID NO: 6 - protein, 28 aa, Artificial Sequence
  Cysteine-Containing “E-coil” Heterodimer- Promoting Domain
EVAACEKEVAALEKEVAALEKEVAALEK

SEQ ID NO: 7 - protein, 28 aa, Artificial Sequence
  Cysteine-Containing “K-coil” Heterodimer- Promoting Domain
KVAACKEKVAALKEKVAALKEKVAALKE

SEQ ID NO: 8 - protein, 217 aa, Artificial Sequence
  IgG1 CH2 and CH3 Domains Having L234A/L235A Substitutions
  MISC_FEATURE (217)..(217): XAA is Lysine (K) or Is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 9 - protein, 217 aa, Artificial Sequence
  IgG1 CH2 and CH3 Domains Having L234A/L235A and M252Y/S254T/T256E Substitutions
  MISC_FEATURE (217)..(217): XAA is Lysine (K) or Is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 10 - protein, 217 aa, Artificial Sequence
  “Knob” Bearing IgG1 CH2 and CH3 Domains Having L234A/L235A Substitutions
  MISC_FEATURE (217)..(217): XAA is Lysine (K) or Is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 11 - protein, 217 aa, Artificial Sequence
  “Hole” Bearing IgG1 CH2 and CH3 Domains Having L234A/L235A Substitutions
  MISC_FEATURE (1)..(217): XAA is Lysine (K) or Is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 12 - protein, 4 aa, Artificial Sequence
  Peptide Linker
GGGS

SEQ ID NO: 13 - protein, 6 aa, Artificial Sequence
  Peptide Linker
GGCGGG

SEQ ID NO: 14 - protein, 5 aa, Artificial Sequence
  Peptide Linker
ASTKG

SEQ ID NO: 15 - protein, 5 aa, Artificial Sequence
  Peptide Linker
APSSS

SEQ ID NO: 16 - protein, 8 aa, Artificial Sequence
  Peptide Linker
APSSSPME

SEQ ID NO: 17 - protein, 6 aa, Artificial Sequence
  Peptide Linker
LEPKSS

SEQ ID NO: 18 - protein, 10 aa, Artificial Sequence
  Peptide Linker
DKTHTCPPCP

SEQ ID NO: 19 - protein, 13 aa, Artificial Sequence
  Peptide Linker
GGGDKTHTCPPCP

SEQ ID NO: 20 - protein, 16 aa, Artificial Sequence
  Peptide Linker
LEPKSSDKTHTCPPCP

SEQ ID NO: 21 - protein, 15 aa, Homo sapiens
  MISC_FEATURE (1)..(15): Exemplary Human IgG1 Hinge Domain
EPKSCDKTHTCPPCP

SEQ ID NO: 22 - protein, 12 aa, Homo sapiens
  MISC_FEATURE (1)..(12): Exemplary Human IgG2 Hinge Domain
ERKCCVECPPCP

SEQ ID NO: 23 - protein, 62 aa, Homo sapiens
  MISC_FEATURE (1)..(62): Exemplary Human IgG3 Hinge Domain
ELKTPLGDTTHTCPRCPEPKSCDTPPPCPRCPEPKSCDTPPPCPRCPEPKSCDTPPPCPRCP

SEQ ID NO: 24 - protein, 12 aa, Homo sapiens
  MISC_FEATURE (1)..(12): Exemplary Human IgG4 Hinge Domain
ESKYGPPCPSCP

SEQ ID NO: 25 - protein, 12 aa, Artificial Sequence
  Exemplary Human IgG4 Hinge Domain Comprising Stabilizing S228P Substitution
ESKYGPPCPPCP

SEQ ID NO: 26 - protein, 110 aa, Artificial Sequence
  Variable Light Chain Domain of Humanized Anti- Human CD3 Antibody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 27 - protein, 14 aa, Artificial Sequence
  CDR1 of Variable Light Chain Domain of Humanized Anti-Human CD3 Antibody
RSSTGAVTTSNYAN

SEQ ID NO: 28 - protein, 7 aa, Artificial Sequence
  CDR2 of Variable Light Chain Domain of Humanized Anti-Human CD3 Antibody
GTNKRAP

SEQ ID NO: 29 - protein, 9 aa, Artificial Sequence
  CDR3 of Variable Light Chain Domain of Humanized Anti-Human CD3 Antibody
ALWYSNLWV

SEQ ID NO: 30 - protein, 120 aa, Artificial Sequence
  Variable Heavy Chain Domain of Humanized Anti- Human CD123 Antibody
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 31 - protein, 5 aa, Artificial Sequence
  CDR1 of Variable Heavy Chain Domain of Humanized Anti-Human CD123 Antibody
DYYMK

SEQ ID NO: 32 - protein, 17 aa, Artificial Sequence
  CDR2 of Variable Heavy Chain Domain of Humanized Anti-Human CD123 Antibody
DIIPSNGATFYNQKFKG

SEQ ID NO: 33 - protein, 10 aa, Artificial Sequence
  CDR3 of Variable Heavy Chain Domain of Humanized Anti-Human CD123 Antibody
SHLLRASFAY

SEQ ID NO: 34 - protein, 113 aa, Artificial Sequence
  Variable Light Chain Domain of Humanized Anti- Human CD123 Antibody
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 35 - protein, 17 aa, Artificial Sequence
  CDR1 of Variable Light Chain Domain of Humanized Anti-Human CD123 Antibody
KSSQSLLNSGNQKNYLT

SEQ ID NO: 36 - protein, 7 aa, Artificial Sequence
  CDR2 of Variable Light Chain Domain of Humanized Anti-Human CD123 Antibody
WASTRES

SEQ ID NO: 37 - protein, 9 aa, Artificial Sequence
  CDR3 of Variable Light Chain Domain of Humanized Anti-Human CD123 Antibody
QNDYSYPYT

SEQ ID NO: 38 - protein, 125 aa, Artificial Sequence
  Variable Heavy Chain Domain of Humanized Anti- Human CD3 Antibody
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 39 - protein, 5 aa, Artificial Sequence
  CDR1 of Variable Heavy Chain Domain of Humanized Anti-Human CD3 Antibody
TYAMN

SEQ ID NO: 40 - protein, 19 aa, Artificial Sequence
  CDR2 of Variable Heavy Chain Domain of Humanized Anti-Human CD3 Antibody
RIRSKYNNYATYYADSVKD

SEQ ID NO: 41 - protein, 14 aa, Artificial Sequence
  CDR3 of Variable Heavy Chain Domain of Humanized Anti-Human CD3 Antibody
HGNFGNSYVSWFAY

SEQ ID NO: 42 - protein, 272 aa, Artificial Sequence
  First Polypeptide Chain of DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 43 - DNA, 816 bp, Artificial Sequence
  Polynucleotide Encoding First Polypeptide Chain of DART-A
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtgcagtccggggctgagctgaagaaacccggagcttccgtgaaggtgtcttgcaaagccagtggctacaccttcacagactactatatgaagtgggtcaggcaggctccaggacagggactggaatggatcggcgatatcattccttccaacggggccactttctacaatcagaagtttaaaggcagggtgactattaccgtggacaaatcaacaagcactgcttatatggagctgagctccctgcgctctgaagatacagccgtgtactattgtgctcggtcacacctgctgagagccagctggtttgcttattggggacagggcaccctggtgacagtgtcttccggaggatgtggcggtggagaagtggccgcactggagaaagaggttgctgctttggagaaggaggtcgctgcacttgaaaaggaggtcgcagccctggagaaa

SEQ ID NO: 44 - protein, 280 aa, Artificial Sequence
  Second Polypeptide Chain of DART-A
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 45 - DNA, 840 bp, Artificial Sequence
  Polynucleotide Encoding Second Polypeptide Chain of DART-A
gacttcgtgatgacacagtctcctgatagtctggccgtgagtctgggggagcgggtgactatgtcttgcaagagctcccagtcactgctgaacagcggaaatcagaaaaactatctgacctggtaccagcagaagccaggccagccccctaaactgctgatctattgggcttccaccagggaatctggcgtgcccgacagattcagcggcagcggcagcggcacagattttaccctgacaatttctagtctgcaggccgaggacgtggctgtgtactattgtcagaatgattacagctatccctacactttcggccaggggaccaagctggaaattaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggaaaagtggccgcactgaaggagaaagttgctgctttgaaagagaaggtcgccgcacttaaggaaaaggtcgcagccctgaaagag

(19) United States
(12) Patent Application Publication
(10) Pub. No.: US 2022/0323583 A1
(43) Pub. Date: Oct. 13, 2022

(54) Pharmaceutical Formulations of Bi-Specific Diabodies and Use of the Same

(71) Applicant: MacroGenics, Inc., Rockville, MD (US)

(72) Inventors: Ian Lent, Rockville, MD (US); Krishnan Sampathkumar, Rockville, MD (US)

(73) Assignee: MacroGenics, Inc., Rockville, MD (US)

(21) Appl. No.: 17/616,830

(22) PCT Filed: May 29, 2020
(86) PCT No.: PCT/US2020/035143

§ 371 Date: Dec. 6, 2021

Related U.S. Application Data
(60) Provisional application No. 62/860,082, filed on Jun. 11, 2019.
(60) Provisional application No. 63/030,010, filed on May 26, 2020.

(51) Int. Cl. A61K 39/395; A61K 47/02; A61K 47/26; C07K 16/28; A61K 47/10; A61K 47/14

(52) U.S. Cl. CPC A61K 39/39591; A61K 47/02; A61K 47/26; C07K 16/2866; C07K 16/2809; A61K 47/10; A61K 47/14; A61K 2039/505

(57) ABSTRACT

The present invention is directed to stable aqueous pharmaceutical formulations that comprise a bispecific monovalent diabody (“Diabody formulation”), and to aqueous stabilizer solutions for stabilizing and administering said diabody. The invention particularly concerns such pharmaceutical formulations that comprise a diabody drug product (DART-A DP formulations) that comprise a sequence-optimized CD 123×CDS bispecific diabody (DART-A) that is capable of simultaneously binding to CD 123 and CDS. The invention further concerns the use of such DART-A DP formulations in the treatment of hematologic malignancies such as acute myeloid leukemia (AML) or myelodysplastic syndrome (MDS) in patients.

### CROSS-REFERENCE TO RELATED APPLICATIONS

This application claims priority to U.S. Patent Applns. Ser. Nos. 62/860,082 (filed on Jun. 11, 2019; pending) and 63/030,010 (filed on May 26, 2020; pending), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0156P2-PCT-TW_ST25.txt, created on May 26, 2020, and having a size of 29,754 bytes), which file is incorporated herein in its entirety.

### FIELD OF THE INVENTION

The present invention is directed to stable aqueous pharmaceutical formulations that comprise a bispecific diabody (“Diabody formulation”), and to aqueous stabilizer solutions for stabilizing and administering said diabody. The invention particularly concerns such pharmaceutical formulations that comprise a diabody drug product (“DART-A DP formulation”) that comprises a sequence-optimized CD123×CD3 bispecific diabody (“DART-A”) that is capable of simultaneously binding to CD123 and CD3. The invention further concerns the use of such DART-A DP formulation and stabilizer in the treatment of hematologic malignancies such as acute myeloid leukemia (AML) or myelodysplastic syndrome (MDS) in patients.

### BACKGROUND OF THE INVENTION

### I. Bispecific Diabodies

Non-monospecific diabodies provide a significant advantage over monospecific natural antibodies because of their capacity to co-ligate and co-localize cells that express different epitopes. Bispecific diabodies have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligation of differing cells, for example, the cross-linking of cytotoxic T cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

Diabody epitope binding domains may also be directed to a surface determinant of an immune effector cell such as CD3, CD16, CD32, or CD64, which are expressed on T lymphocytes, natural killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; and PCT Publn. Nos. WO 2006/113665; WO 2008/157379; WO 2010/080538; WO 2012/018687; and WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody brings the effector cell within the proximity of the tumor cells and leads to effective tumor killing (see e.g., Cao et al. (2003) “Bispecific Antibody Conjugates In Therapeutics,” Adv. Drug. Deliv. Rev. 55:171-197).

However, the above advantages come at a salient cost. The formation of such non-monospecific diabodies requires the successful assembly of two or more distinct and different polypeptides (i.e., such formation requires that the diabodies be formed through the heterodimerization of different polypeptide chain species). In contrast, mono-specific diabodies are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8): 583-588), and promotenon-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, stable, covalently bonded heterodimeric non-monospecific diabodies have been developed (see, e.g., PCT Publn. Nos. WO 2006/113665; WO/2008/157379; WO 2010/080538; WO 2012/018687; and WO/2012/162068; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7): 1933-1943; Moore, P. A. et al. (2011) “Application Of Dual Affinity Retargeting Molecules To Achieve Optimal Redirected T-Cell Killing Of B-Cell Lymphoma,” Blood 117(17):4542-4551). Such approaches involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

The production of stable, functional heterodimeric, non-monospecific diabodies can be further optimized by the careful consideration and placement of heterodimer-promoting domains and cysteine residues in one or more of the employed polypeptide chains. Such optimized diabodies can be produced in higher yield and with greater activity than non-optimized diabodies. The present invention is directed to the problem of providing formulations and stabilizer solutions that are particularly designed and optimized for storage and administration of such heterodimeric diabodies. The invention solves this problem through the provision of exemplary diabody formulations and stabilizer solutions, useful for administration of bispecific diabodies, particularly those administered at low doses (e.g., about 1 μg/kg or less) and/or requiring the preparation of dosing solutions comprising low concentrations (e.g., about 5 μg/mL or less), such as optimized CD123×CD3 diabodies.

### II. CD123

CD123 (interleukin 3 receptor alpha, IL-3Ra) is a 40 kDa molecule and is part of the interleukin 3 receptor complex (Stomski, F. C. et al. (1996) “Human Interleukin-(IL-3) Induces Disulfide-Linked IL-3 Receptor Alpha-And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 is expressed on CD34+ committed progenitors (Taussig, D. C. et al. (2005) “Hematopoietic Stem Cells Express Multiple Myeloid Markers: Implications For The Origin And Targeted Therapy Of Acute Myeloid Leukemia,” Blood 106:4086-4092), but not by CD34+/CD38− normal hematopoietic stem cells. CD123 is expressed by basophils, mast cells, plasmacytoid dendritic cells, some expression by monocytes, macrophages and eosinophils, and low or no expression by neutrophils and megakaryocytes. Some non-hematopoietic tissues (placenta, Leydig cells of the testis, certain brain cell elements and some endothelial cells) express CD123; however, expression is mostly cytoplasmic.

CD123 is reported to be expressed by leukemic blasts and leukemia stem cells (LSC) (Jordan, C. T. et al. (2000) “The Interleukin-3 Receptor Alpha Chain Is A Unique Marker For Human Acute Myelogenous Leukemia Stem Cells,” Leukemia 14:1777-1784; Jin, W. et al. (2009) “Regulation Of Th17 Cell Differentiation And EAE Induction By MAP3K NIK,” Blood 113:6603-6610). In human normal precursor populations, CD123 is expressed by a subset of hematopoietic progenitor cells (HPC) but not by normal hematopoietic stem cells (HSC). CD123 is also expressed by plasmacytoid dendritic cells (pDC) and basophils, and, to a lesser extent, monocytes and eosinophils (Lopez, A. F. et al. (1989) “Reciprocal Inhibition Of Binding Between Interleukin 3 And Granulocyte-Macrophage Colony-Stimulating Factor To Human Eosinophils,” Proc. Natl. Acad. Sci. (U.S.A.) 86:7022-7026; Sun, Q. et al. (1996) “Monoclonal Antibody 7G3 Recognizes The N-Terminal Domain Of The Human Interleukin-3 (IL-3) Receptor Alpha Chain And Functions As A Specific IL-3 Receptor Antagonist,” Blood 87:83-92; Munoz, L. et al. (2001) “Interleukin-3 Receptor Alpha Chain (CD123) Is Widely Expressed In Hematologic Malignancies,” Haematologica 86(12):1261-1269; Masten, B. J. et al. (2006) “Characterization Of Myeloid And Plasmacytoid Dendritic Cells In Human Lung,” J. Immunol. 177:7784-7793; Korpelainen, E. I. et al. (1995) “Interferon-Gamma Upregulates Interleukin-3 (IL-3) Receptor Expression In Human Endothelial Cells And Synergizes With IL-3 In Stimulating Major Histocompatibility Complex Class II Expression And Cytokine Production,” Blood 86:176-182).

CD123 has been reported to be overexpressed on malignant cells in a wide range of hematologic malignancies including AML and MDS (Muñoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine Induced Killer Cells Redirected With A Novel CD123-Specific Chimeric Antigen Receptor,” Br. J. Haematol. 161:389-401).

### III. CD3

CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pp. 1-14). In mammals, the complex contains a CD3γ chain, a CD3δ chain, and two CD3c chains. These chains associate with a molecule known as the T cell receptor (TCR) to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139).

### IV. AML and MDS

AML and MDS are thought to arise in, and be perpetuated by, a small population of leukemic stem cells (LSCs), which are generally dormant (i.e., not rapidly dividing cells) and therefore resist cell death (apoptosis) and conventional chemotherapeutic agents. LSCs are characterized by high levels of CD123 expression, which is not present in the corresponding normal HSC population in normal human bone marrow (Jin, W. et al. (2009) “Regulation Of Th17 Cell Differentiation And EAE Induction By MAP3K NIK,” Blood 113:6603-6610; Jordan, C. T. et al. (2000) “The Interleukin-3 Receptor Alpha Chain Is A Unique Marker For Human Acute Myelogenous Leukemia Stem Cells,” Leukemia 14:1777-1784). CD123 is expressed in 45%-95% of AML, 85% of Hairy cell leukemia (HCL), and 40% of acute B lymphoblastic leukemia (B-ALL). CD123 expression is also associated with multiple other malignancies/pre-malignancies: chronic myeloid leukemia (CML) progenitor cells (including blast crisis CML); Hodgkin's Reed Sternberg (RS) cells; transformed non-Hodgkin's lymphoma (NHL); some chronic lymphocytic leukemia (CLL) (CD11c+); a subset of acute T lymphoblastic leukemia (T-ALL) (16%, most immature, mostly adult), pDC DC2 malignancies and CD34+/CD38− MDS marrow cell malignancies.

AML is a clonal disease characterized by the proliferation and accumulation of transformed myeloid progenitor cells in the bone marrow, which ultimately leads to hematopoietic failure. The incidence of AML increases with age, and older patients typically have worse treatment outcomes than do younger patients (Robak, T. et al. (2009) “Current And Emerging Therapies For Acute Myeloid Leukemia,” Clin. Ther. 2:2349-2370). Unfortunately, at present, most adults with AML die from their disease.

Treatment for AML initially focuses on the induction of remission (induction therapy). Once remission is achieved, treatment shifts to focus on securing such remission (post-remission or consolidation therapy) and, in some instances, maintenance therapy. The standard remission induction paradigm for AML is chemotherapy with an anthracycline/cytarabine combination, followed by either consolidation chemotherapy (usually with higher doses of the same drugs as were used during the induction period) or human stem cell transplantation, depending on the patient's ability to tolerate intensive treatment and the likelihood of cure with chemotherapy alone (see, e.g., Roboz, G. J. (2012) “Current Treatment Of Acute Myeloid Leukemia,” Curr. Opin. Oncol. 24:711-719).

Agents frequently used in induction therapy include cytarabine (also known as AraC) and the anthracyclines. AraC kills cancer cells (and other rapidly dividing normal cells) by interfering with DNA synthesis. Side effects associated with AraC treatment include decreased resistance to infection, a result of decreased white blood cell production; bleeding, as a result of decreased platelet production; and anemia, due to a potential reduction in red blood cells. Other side effects include nausea and vomiting. Anthracyclines (e.g., daunorubicin, doxorubicin, and idarubicin) have several modes of action including inhibition of DNA and RNA synthesis, disruption of higher order structures of DNA, and production of cell damaging free oxygen radicals. The most consequential adverse effect of anthracyclines is cardiotoxicity, which considerably limits administered life-time dose and to some extent their usefulness.

Thus, unfortunately, despite substantial progress in the treatment of newly diagnosed AML, 20% to 40% of patients do not achieve remission with the standard induction chemotherapy, and 50% to 70% of patients entering a first complete remission are expected to relapse within 3 years. The optimum strategy at the time of relapse, or for patients with the resistant disease, remains uncertain. Stem cell transplantation has been established as the most effective form of anti-leukemic therapy in patients with AML in first or subsequent remission (Roboz, G. J. (2012) “Current Treatment Of Acute Myeloid Leukemia,” Curr. Opin. Oncol. 24:711-719).

### SUMMARY OF THE INVENTION

The present invention is directed to stable aqueous pharmaceutical formulations that comprise a bispecific diabody (Diabody formulation), and to aqueous stabilizer solutions for stabilizing said diabody. The invention particularly concerns such pharmaceutical formulations that comprise a diabody drug product (DART-A DP formulation) that comprise a sequence-optimized CD123×CD3 bi-specific diabody (DART-A) that is capable of simultaneously binding to CD123 and CD3. The invention further concerns the use of such DART-A DP formulation and stabilizers in the treatment of hematologic malignancies such as AML or MDS in patients.

In detail, the invention provides a stable aqueous pharmaceutical formulation comprising a diabody (e.g., a CD123×CD3 diabody), a sodium phosphate buffer, sodium chloride, and polysorbate 80 (“PS80”).

The invention additionally provides the embodiment of such stable aqueous pharmaceutical formulation wherein the sodium phosphate has a concentration of about 5 mM to about 30 mM, and especially wherein the concentration of sodium phosphate is about 10 mM.

The invention additionally provides the embodiment of such stable aqueous pharmaceutical formulation wherein the PS80 has a concentration of about 0.05 mg/mL to about 0.3 mg/mL, and especially wherein the PS80 has a concentration about 0.1 mg/mL.

The invention additionally provides the embodiment of such stable aqueous pharmaceutical formulations wherein the sodium chloride has a concentration of about 100 mM to about 300 mM, and especially about 150 mM.

The invention additionally provides the embodiment of such stable aqueous pharmaceutical formulations wherein the formulation has a pH of about 5.5 to about 7.0, and especially a pH of about 6.0.

The invention additionally provides the embodiment of such stable aqueous pharmaceutical formulations wherein the formulation comprises about 10 mM sodium phosphate, about 150 mM sodium chloride, and about 0.1 mg/mL PS80, and wherein the formulation has a pH of about 6.0.

The invention additionally provides the embodiment of such stable aqueous pharmaceutical formulations wherein the diabody has a concentration of about 0.01 mg/mL to about 1 mg/mL, and especially about 0.1 mg/mL.

The invention particularly concerns the embodiment of such stable aqueous pharmaceutical formulations wherein the diabody is a covalently bonded bispecific diabody comprising two, three, or four polypeptide chains. The invention additionally provides such stable aqueous pharmaceutical formulations, wherein the covalently bonded diabody is a CD123×CD3 diabody and more particularly wherein the CD123×CD3 diabody comprises:
a) a first polypeptide chain having the amino acid sequence of SEQ ID NO:42; and
b) a second polypeptide chain having the amino acid sequence of SEQ ID NO:44; and
   wherein the first and the second polypeptide chains are covalently bonded to one another by a disulfide bond.

The invention additionally provides the embodiment of such stable aqueous pharmaceutical formulations wherein the formulation comprises about 0.1 mg/mL of the diabody, about 10 mM sodium phosphate, about 150 mM sodium chloride, and about 0.1 mg/mL PS80, and wherein the pH of the formulation is about 6.0.

The invention additionally provides a container comprising any of the above-described stable aqueous pharmaceutical formulations, and especially, wherein such container is a glass vial that is asceptically filled.

The invention additionally provides the embodiment of such stable aqueous pharmaceutical formulations aseptically filled in vials wherein the solution maintains monomeric purity of the diabody for about 3 months at 25° C.

The invention additionally provides the embodiment of such stable aqueous pharmaceutical formulations aseptically filled in vials wherein the solution maintains monomeric purity of the diabody for about 48 months at 2⁻8° C.

The invention additionally provides a sealed package comprising any of the above-described stable aqueous pharmaceutical formulations.

The invention additionally provides an aqueous stabilizer solution for stabilizing a diabody comprising sodium phosphate, PS80, benzyl alcohol (“BA”), and methylparaben (“MP”).

The invention additionally provides an aqueous stabilizer solution wherein the sodium phosphate has a concentration of about 15 mM to about 25 mM, and especially wherein the concentration of sodium phosphate is about 20 mM.

The invention additionally provides such an aqueous stabilizer solution wherein the BA has a concentration of about 11.5 mg/mL to about 15.5 mg/mL, and especially wherein the concentration of BA is about 13.2 mg/mL.

The invention additionally provides such aqueous stabilizer solutions wherein the MP has a concentration of about 3.5 mg/mL to about 5.5 mg/mL, and especially wherein the concentration of MP is about 4.25 mg/mL.

The invention additionally provides the embodiment of any of such aqueous stabilizer solutions wherein the PS80 has a concentration of about 0.1 mg/mL to about 0.4 mg/mL, and especially wherein the concentration of PS80 is about 0.25 mg/mL

The invention additionally provides the embodiment of any of such aqueous stabilizer solutions wherein the solution has a pH of about 7.7 to about 8.7, and especially wherein the pH is about 8.2.

The invention additionally provides the embodiment of such an aqueous stabilizer solution wherein the stabilizer solution comprises about 20 mM sodium phosphate, about 13.2 mg/mL BA, about 4.25 mg/mL MP and about 0.25 mg/mL PS80, and wherein the pH of the solution is about 8.2.

The invention particularly concerns such an aqueous stabilizer solution wherein the diabody is a covalently bonded bispecific diabody comprising two, three, or four polypeptide chains.

The invention additionally provides such aqueous stabilizer solutions, wherein the covalently bonded diabody is a CD123×CD3 diabody and more particularly wherein the CD123×CD3 diabody comprises:
a) a first polypeptide chain having the amino acid sequence of SEQ ID NO:42; and
b) a second polypeptide chain having the amino acid sequence of SEQ ID NO:44; and
   wherein the first and the second polypeptide chains are covalently bonded to one another by a disulfide bond.

The invention additionally provides the embodiment of such aqueous stabilizer solutions wherein the solution maintains monomeric purity of the diabody for about 3-5 days at about 25° C.

The invention additionally provides the embodiment of such aqueous stabilizer solutions wherein the solution maintains monomeric purity of the diabody for about 5-7 days at about 25° C.

The invention additionally provides the embodiment of such aqueous stabilizer solutions wherein the solution inhibits or prevents microbial growth for about 3-5 days at about 25° C.

The invention additionally provides the embodiment of such aqueous stabilizer solutions wherein the solution prevents microbial growth for about 5-7 days at about 25° C.

The invention additionally provides the embodiment of such aqueous stabilizer solutions in vials wherein the solution has a shelf-life of at least about 2 years at 2-8° C. or at least about 3 months at 25° C.

The invention additionally provides a container comprising any of the above-described stable aqueous stabilizer solutions, and especially, wherein such container is a glass vial that is aseptically filled.

The invention additionally provides a sealed package comprising any of such aqueous stabilizer solutions.

The invention additionally provides an aqueous stabilizer solution for stabilizing a diabody, comprising sodium chloride and PS80.

The invention additionally provides the embodiment of such aqueous stabilizer solutions wherein the sodium chloride has a concentration of about 100 mM to about 300 mM, and especially about 150 mM.

The invention additionally provides the embodiment of such aqueous stabilizer solutions wherein the PS80 has a concentration of about 0.05 mg/mL to about 0.3 mg/mL, and especially about 0.10 mg/mL.

The invention additionally provides the embodiment of such aqueous stabilizer solutions wherein the solution has a pH of about 5.5 to about 7.0, and especially wherein the pH is 6.0.

The invention additionally provides the embodiment of such aqueous stabilizer solutions wherein the solution comprises about 150 mM sodium chloride, about 0.10 mg/mL PS80 and wherein the pH of the solution is about 6.0.

The invention particularly concerns such an aqueous stabilizer solution wherein the diabody is a covalently bonded bispecific diabody comprising two, three, or four polypeptide chains.

The invention additionally provides such aqueous stabilizer solutions, wherein the covalently bonded diabody is a CD123×CD3 diabody and more particularly wherein the CD123×CD3 diabody comprises:
a) a first polypeptide chain having the amino acid sequence of SEQ ID NO:42; and
b) a second polypeptide chain having the amino acid sequence of SEQ ID NO:44; and
   wherein the first and the second polypeptide chains are covalently bonded to one another by a disulfide bond.

The invention additionally provides the embodiment of such aqueous stabilizer solutions wherein the solution maintains monomeric purity of the diabody for about 3-5 days at about 25° C.

The invention additionally provides the embodiment of such aqueous stabilizer solutions wherein the solution maintains monomeric purity of the diabody for about 5-7 days at about 25° C.

The invention additionally provides the embodiment of such aqueous stabilizer solutions wherein the solution inhibits or prevents microbial growth for about 3-5 days at about 25° C.

The invention additionally provides the embodiment of such aqueous stabilizer solutions wherein the solution prevents microbial growth for about 5-7 days at about 25° C.

The invention additionally provides the embodiment of such aqueous stabilizer solutions in vials wherein the solution has a shelf-life of at least about 2 years at 2-8° C. or at least about 3 months at 25° C.

The invention additionally provides a container comprising any of the above-described stable aqueous stabilizer solutions, and especially, wherein such container is a glass vial that is aseptically filled.

The invention additionally provides a sealed package comprising any of such aqueous stabilizer solutions.

The invention additionally provides an aqueous stabilizer solution for stabilizing a diabody, comprising sodium phosphate, sodium chloride, a PS80, and a BA. The invention additionally provides the embodiment of such aqueous stabilizer solution wherein the sodium phosphate has a concentration of about 5 mM to about 30 mM.

The invention additionally provides the embodiment of such aqueous stabilizer solutions wherein the concentration of the sodium phosphate is about 10 mM.

The invention additionally provides the embodiment of such aqueous stabilizer solutions wherein the sodium chloride has a concentration of about 100 mM to about 300 mM.

The invention additionally provides the embodiment of such aqueous stabilizer solutions wherein the concentration of the sodium chloride is about 150 mM.

The invention additionally provides the embodiment of such aqueous stabilizer solutions wherein the BA has a concentration of about 7.0 mg/mL to about 11.0 mg/mL.

The invention additionally provides the embodiment of such aqueous stabilizer solutions wherein the concentration of the BA is about 9.0 mg/mL.

The invention additionally provides the embodiment of such aqueous stabilizer solutions wherein the PS80 has a concentration of about 0.05 mg/mL to about 0.3 mg/mL.

The invention additionally provides the embodiment of such aqueous stabilizer solutions wherein the concentration of the PS80 is about 0.10 mg/mL.

The invention additionally provides the embodiment of such aqueous stabilizer solutions further comprising recombinant human albumin (“rHA”).

The invention additionally provides the embodiment of such aqueous stabilizer solutions wherein the rHA has a concentration of about 0.05 mg/mL to about 0.15 mg/mL, and especially wherein the concentration of the rHA is about 0.10 mg/mL.

The invention additionally provides the embodiment of such aqueous stabilizer solutions wherein the solution has a pH of about 5.5 to about 7.0, and especially wherein the pH is 6.0.

The invention particularly provides the embodiment of such aqueous stabilizer solutions wherein the solution comprises about 10 mM sodium phosphate, about 150 mM sodium chloride, about 9.0 mg/mL BA, about 0.1 mg/mL PS80, and about 0.1 mg/mL rHA, and wherein the pH of the solution is about 6.0.

The invention particularly provides the embodiment of such aqueous stabilizer solutions wherein the solution comprises about 10 mM sodium phosphate, about 150 mM sodium chloride, about 0.1 mg/mL PS80 and wherein the pH of the solution is about 6.0.

The invention additionally provides the embodiment of such aqueous stabilizer solutions wherein the diabody is a covalently bonded bispecific diabody.

The invention additional provides such aqueous stabilizer solutions wherein the covalently bonded diabody is a CD123×CD3 diabody and more particularly, wherein the CD123×CD3 diabody comprises:
a) a first polypeptide chain having the amino acid sequence of SEQ ID NO:42; and
b) a second polypeptide chain having the amino acid sequence of SEQ ID NO:44; and
   wherein the first and the second polypeptide chains are covalently bonded to one another by a disulfide bond.

The invention additionally provides the embodiment of such aqueous stabilizer solutions wherein the solution maintains monomeric purity of the diabody for about 3 days at about 25° C.

The invention additionally provides the embodiment of such aqueous stabilizer solutions wherein the solution inhibits or prevents microbial growth for about 3-5 days at about 25° C.

The invention additionally provides the embodiment of such aqueous stabilizer solutions wherein the solution has a shelf-life of at least about 2 years at 2-8° C. or at least about 3 months at 25° C.

The invention additionally provides a container (especially a glass vial container) comprising any of such aqueous stabilizer solutions.

The invention additionally provides a sealed package comprising any of such aqueous stabilizer solutions.

The invention additionally provides a kit comprising:
a) a container A comprising a stable aqueous pharmaceutical formulation, the formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and the formulation has a pH of about 5.5 to about 7.0; and
b) a container B comprising an aqueous stabilizer solution for stabilizing the diabody, the solution comprising about 15 mM to about 25 mM sodium phosphate buffer, about 11.5 mg/mL to about 15.5 BA, about 3.5 mg/mL to about 5.5 mg/mL MP and about 0.1 mg/mL to about 0.4 mg/mL PS80, and the solution has a pH of about 7.7 to about 8.7; and optionally
c) instructions for combining the contents of containers A and B for administration of the combined solution to a subject in need thereof.

The invention additionally provides a kit comprising:
a) a container A comprising a stable aqueous pharmaceutical formulation, the formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and the formulation has a pH of about 5.5 to about 7.0; and
b) (i) a container B comprising an aqueous stabilizer solution for stabilizing the diabody, the solution comprising about 5 mM to about 30 mM sodium phosphate, about 100 mM to about 300 mM sodium chloride, about 7.0 mg/mL to about 11.0 mg/mL BA, about 0.05 mg/mL to about 0.3 mg/mL PS80, and the solution has a pH of about 5.5 to about 7.0; or
   (ii) a container B comprising an aqueous stabilizer solution for stabilizing the diabody, the solution comprising about 5 mM to about 30 mM sodium phosphate, about 100 mM to about 300 mM sodium chloride, about 0.05 mg/mL to about 0.3 mg/mL PS80, and the solution has a pH of about 5.5 to about 7.0; or
   (iii) a container B comprising an aqueous stabilizer solution for stabilizing the diabody, the solution comprising about 100 mM to about 300 mM sodium chloride, about 0.05 mg/mL to about 0.3 mg/mL PS80, and the solution has a pH of about 5.5 to about 7.0; and optionally,
c) instructions for combining the contents of containers A and B for administration of the combined solution to a subject in need thereof.

The invention additionally provides the embodiment of such kits wherein the diabody is a covalently bonded bispecific diabody comprising two, three, or four polypeptide chains.

The invention additional provides such kits wherein the covalently bonded diabody is a CD123×CD3 diabody and more particularly wherein the CD123×CD3 diabody comprises:
a) a first polypeptide chain having the amino acid sequence of SEQ ID NO:42; and
b) a second polypeptide chain having the amino acid sequence of SEQ ID NO:44; and
   wherein the first and the second polypeptide chains are covalently bonded to one another by a disulfide bond.

The invention additionally provides the embodiment of such kits wherein the stable aqueous pharmaceutical formulation in container A comprises about 0.1 mg/mL of the diabody, about 10 mM sodium phosphate, about 150 mM sodium chloride, and about 0.1 mg/mL PS80, and wherein the formulation has a pH of about 6.0.

The invention additionally provides the embodiment of such kits wherein the aqueous stabilizer solution in container B comprises about 20 mM sodium phosphate, about 13.2 mg/mL BA, about 4.25 mg/mL MP and about 0.25 mg/mL PS80, and

wherein the solution has a pH of about 8.2.

The invention additionally provides the embodiment of such kits wherein the aqueous stabilizer solution in container B comprises about 10 mM sodium phosphate, about 150 mM sodium chloride, about 9.0 mg/mL BA, about 0.1 mg/mL PS80, and wherein the solution has a pH of about 6.0.

The invention additionally provides the embodiment of such kits wherein the aqueous stabilizer solution in container B comprises about 10 mM sodium phosphate, about 150 mM sodium chloride, about 0.1 mg/mL PS80, and wherein the solution has a pH of about 6.0.

The invention additionally provides the embodiment of such kits wherein the aqueous stabilizer solution in container B comprises about 150 mM sodium chloride, about 0.10 mg/mL PS80, and wherein the solution has a pH of about 6.0.

The invention additionally provides the embodiment of such kits wherein the aqueous stabilizer solution further comprises rHA at a concentration of about 0.05 mg/mL to about 0.3 mg/mL.

The invention additionally provides the embodiment of such kits wherein the concentration of rHA is about 0.1 mg/mL.

The invention additionally provides the embodiment of such kits wherein the subject is a human patient.

The invention additionally provides the embodiment of such kits wherein the container A and container B are glass vials.

The invention additionally provides a sealed package comprising any of the above-described kits, and optionally instructions for storage and/or use of such kit.

The invention additionally provides a method of administering a diabody to a subject in need thereof comprising using one of the above-described kits wherein the aqueous stabilizer solution of the container B comprises the sodium phosphate, PS80, BA, MP, and has a pH of about 7.7 to about 8.7;

and wherein in the method:
(a) the aqueous stabilizer solution of container B is placed in a container C and mixed;
(b) the stable aqueous pharmaceutical formulation of container A is placed in the container C and mixed to obtain a dosing solution;
(c) the container C containing the dosing solution is attached to a device for administration to the subject.

The invention additionally provides the embodiment of such methods wherein the container C comprises saline for intravenous infusion.

The invention additionally provides a method of administering a diabody to a subject in need thereof using one of the above-described kits wherein the aqueous stabilizer solution of the container B comprises one or more of sodium phosphate, sodium chloride, PS80, BA, and optionally rHA, and has a pH of about 5.5 to about 7.0;

and wherein in the method:
(a) the stable aqueous pharmaceutical formulation of the container A is placed in the aqueous stabilizer solution of the container B and mixed to obtain a dosing solution;
(b) optionally diluting the dosing solution;
(c) placing the dosing solution in a container C; and
(d) attaching the container C containing the final dosing solution to a device for administration to the subject.

The invention additionally provides the embodiment of such methods wherein the container C comprises saline or bacteriostatic saline for intravenous infusion.

The invention additionally provides the embodiment of such methods where the administration is by an infusion pump.

The invention additionally provides the embodiment of such methods wherein the administration is ambulatory.

The invention additionally provides the embodiment of such methods wherein the device is a single ambulatory pump.

The invention additionally provides the embodiment of such methods wherein the device is a dual ambulatory pump.

The invention additionally provides the embodiment of such methods wherein the device is a syringe pump.

The invention additionally provides the embodiment of such methods wherein the administration is by continuous infusion for at least about 24 hours.

The invention additionally provides the embodiment of such methods wherein the administration is by continuous infusion for at least about 48 hours.

The invention additionally provides the embodiment of such methods wherein the administration is by continuous infusion for at least about 96 hours.

The invention additionally provides the embodiment of such methods wherein the administration is by continuous infusion for at least about 7 days.

The invention additionally provides the embodiment of such methods wherein the administration occurs at a flow rate of about 0.10 mL/hour to about 2.5 mL/hour.

The invention additionally provides the embodiment of such methods wherein the administration occurs at a flow rate of about 0.5 mL/hour to about 10.0 mL/hour.

The invention additionally provides the embodiment of such methods wherein the administration is by continuous infusion for at least 24 hours at a flow rate of about 0.1 mL/hour to about 2.0 mL/hour.

The invention additionally provides the embodiment of such methods wherein the administration is by continuous infusion for at least 48 hours at a flow rate of about 0.5 mL/hour to about 6 mL/hour.

The invention additionally provides the embodiment of such methods wherein the administration is by continuous infusion for at least 96 hours at a flow rate of about 0.6 mL/hour to about 3.0 mL/hour.

The invention additionally provides the embodiment of such methods wherein the administration is by continuous infusion for at least 96 hours at a flow rate of about 0.3 mL/hour to about 3.0 mL/hour.

The invention additionally provides the embodiment of such methods wherein the administration is by continuous infusion for at least 96 hours at a flow rate of about 0.5 mL/hour.

The invention additionally provides the embodiment of such methods wherein the administration is by continuous infusion for at least 7 days at a flow rate of about 0.3 mL/hour to about 3.0 ml/hour.

The invention additionally provides the embodiment of such methods wherein the administration is by continuous infusion for at least 7 days at a flow rate of about 0.5 mL/hour.

The invention additionally provides the embodiment of such methods wherein the flow rate prevents vein occlusion in the subject.

The invention additionally provides the embodiment of such methods wherein the diabody is a CD123×CD3 diabody and is administered to the subject at a treatment dosage selected from the group consisting of 30-500 ng/kg/day.

The invention additionally provides the embodiment of such methods wherein the dosing solution comprises 40 mL of aqueous stabilizer solution.

The invention additionally provides the embodiment of such methods wherein the dosing solution comprises about 0.03 mg/mL to about 0.04 mg/mL PS80, about 1.7 mg/mL to about 2.1 mg/mL BA, and about 0.55 mg/mL to about 0.7 mg/mL MP.

The invention additionally provides the embodiment of such methods wherein said dosing solution comprises about 100 mM to about 300 mM sodium chloride, about 0.05 mg/mL to about 0.15 mg/mL PS80, and said solution has a pH of about 5.5 to about 7.0.

The invention additionally provides the embodiment of such methods wherein the patient is a human subject.

The invention additionally provides a method of treating a hematologic malignancy comprising administering to a subject in need thereof, a therapeutically effective amount of a dosing solution comprising:
(A) (1) a stable aqueous pharmaceutical formulation comprising a CD123×CD3 diabody, a sodium phosphate buffer, sodium chloride, and PS80, and an aqueous stabilizer solution that comprises sodium phosphate, PS80, BA, and MP; or
   (2) a stable aqueous pharmaceutical formulation comprising a CD123×CD3 diabody, a sodium phosphate buffer, sodium chloride, and PS80, wherein said sodium phosphate has a concentration of about 5 mM to about 30 mM, and an aqueous stabilizer solution that comprises sodium phosphate, PS80, BA, and MP; or
   (3) a stable aqueous pharmaceutical formulation comprising a CD123×CD3 diabody, a sodium phosphate buffer, sodium chloride, and PS80, wherein said sodium phosphate has a concentration of about 10 mM, and an aqueous stabilizer solution that comprises sodium phosphate, PS80, BA, and MP; or
   (4) a stable aqueous pharmaceutical formulation comprising a CD123×CD3 diabody, a sodium phosphate buffer, sodium chloride, and PS80, wherein said PS80 has a concentration of about 0.05 mg/mL to about 0.3 mg/mL, and an aqueous stabilizer solution that comprises sodium phosphate, PS80, BA, and MP; or
   (5) a stable aqueous pharmaceutical formulation comprising a CD123×CD3 diabody, a sodium phosphate buffer, sodium chloride, and PS80, wherein said PS80 has a concentration of about 0.1 mg/mL, and an aqueous stabilizer solution that comprises sodium phosphate, PS80, BA, and MP; or
   (6) a stable aqueous pharmaceutical formulation comprising a CD123×CD3 diabody, a sodium phosphate buffer, sodium chloride, and PS80, wherein said sodium chloride has a concentration of about 100 mM to about 300 mM, and an aqueous stabilizer solution that comprises sodium phosphate, PS80, BA, and MP; or
   (7) a stable aqueous pharmaceutical formulation comprising a CD123×CD3 diabody, a sodium phosphate buffer, sodium chloride, and PS80, wherein said sodium chloride has a concentration of about 150 mM, and an aqueous stabilizer solution that comprises sodium phosphate, PS80, BA, and MP; or
   (8) a stable aqueous pharmaceutical formulation comprising a CD123×CD3 diabody, a sodium phosphate buffer, sodium chloride, and PS80, wherein said formulation has a pH of about 5.5 to about 7.0, and an aqueous stabilizer solution that comprises sodium phosphate, PS80, BA, and MP; or
   (9) a stable aqueous pharmaceutical formulation comprising a CD123×CD3 diabody, a sodium phosphate buffer, sodium chloride, and PS80, wherein said formulation has a pH of about 6.0, and an aqueous stabilizer solution that comprises sodium phosphate, PS80, BA, and MP; or
   (10) a stable aqueous pharmaceutical formulation comprising a CD123×CD3 diabody, a sodium phosphate buffer, sodium chloride, and PS80, wherein said CD123×CD3 diabody has a concentration of about 0.01 mg/mL to about 1 mg/mL, and an aqueous stabilizer solution that comprises sodium phosphate, PS80, BA, and MP; or
   (11) a stable aqueous pharmaceutical formulation comprising a CD123×CD3 diabody, a sodium phosphate buffer, sodium chloride, and PS80, wherein said concentration of CD123×CD3 diabody is about 0.1 mg/mL, and an aqueous stabilizer solution that comprises sodium phosphate, PS80, BA, and MP; or
   (12) a stable aqueous pharmaceutical formulation comprising a CD123×CD3 diabody, a sodium phosphate buffer, sodium chloride, and PS80, wherein said CD123×CD3 diabody comprises:
      a) a first polypeptide chain having the amino acid sequence of SEQ ID NO:42; and
      b) a second polypeptide chain having the amino acid sequence of SEQ ID NO:44; and
      wherein the first and said second polypeptide chains are covalently bonded to one another by a disulfide bond, and an aqueous stabilizer solution that comprises sodium phosphate, PS80, BA, and MP; or
   (13) a stable aqueous pharmaceutical formulation comprising a CD123×CD3 diabody, a sodium phosphate buffer, sodium chloride, and PS80, wherein said formulation comprises about 0.1 mg/mL of said CD123×CD3 diabody, about 10 mM sodium phosphate, about 150 mM sodium chloride, and about 0.1 mg/mL PS80, and wherein the pH of said formulation is about 6.0, and an aqueous stabilizer solution that comprises sodium phosphate, PS80, BA, and MP; or
   (14) a stable aqueous pharmaceutical formulation comprising a CD123×CD3 diabody, a sodium phosphate buffer, sodium chloride, and PS80, wherein said solution maintains monomeric purity of said CD123×CD3 diabody for about 3 months at 25° C., and an aqueous stabilizer solution that comprises sodium phosphate, PS80, BA, and MP; or
   (15) a stable aqueous pharmaceutical formulation comprising a CD123×CD3 diabody, a sodium phosphate buffer, sodium chloride, and PS80, wherein said solution maintains monomeric purity of said CD123×CD3 diabody for about 48 months at 2-8° C., and an aqueous stabilizer solution that comprises sodium phosphate, PS80, BA, and MP; or
(B) (1) a stable aqueous pharmaceutical formulation comprising a CD123×CD3 diabody, a sodium phosphate buffer, sodium chloride, and PS80, and an aqueous stabilizer solution that comprises sodium phosphate, sodium chloride, a PS80, and a BA; or
   (2) a stable aqueous pharmaceutical formulation comprising a CD123×CD3 diabody, a sodium phosphate buffer, sodium chloride, and PS80, wherein said sodium phosphate has a concentration of about 5 mM to about 30 mM, and an aqueous stabilizer solution that comprises sodium phosphate, sodium chloride, a PS80, and a BA; or
   (3) a stable aqueous pharmaceutical formulation comprising a CD123×CD3 diabody, a sodium phosphate buffer, sodium chloride, and PS80, wherein said sodium phosphate has a concentration of about 10 mM, and an aqueous stabilizer solution that comprises sodium phosphate, sodium chloride, a PS80, and a BA; or
   (4) a stable aqueous pharmaceutical formulation comprising a CD123×CD3 diabody, a sodium phosphate buffer, sodium chloride, and PS80, wherein said PS80 has a concentration of about 0.05 mg/mL to about 0.3 mg/mL, and an aqueous stabilizer solution that comprises sodium phosphate, sodium chloride, a PS80, and a BA; or
   (5) a stable aqueous pharmaceutical formulation comprising a CD123×CD3 diabody, a sodium phosphate buffer, sodium chloride, and PS80, wherein said PS80 has a concentration of about 0.1 mg/mL, and an aqueous stabilizer solution that comprises sodium phosphate, sodium chloride, a PS80, and a BA; or
   (6) a stable aqueous pharmaceutical formulation comprising a CD123×CD3 diabody, a sodium phosphate buffer, sodium chloride, and PS80, wherein said sodium chloride has a concentration of about 100 mM to about 300 mM, and an aqueous stabilizer solution that comprises sodium phosphate, sodium chloride, a PS80, and a BA; or
   (7) a stable aqueous pharmaceutical formulation comprising a CD123×CD3 diabody, a sodium phosphate buffer, sodium chloride, and PS80, wherein said sodium chloride has a concentration of about 150 mM, and an aqueous stabilizer solution that comprises sodium phosphate, sodium chloride, a PS80, and a BA; or
   (8) a stable aqueous pharmaceutical formulation comprising a CD123×CD3 diabody, a sodium phosphate buffer, sodium chloride, and PS80, wherein said formulation has a pH of about 5.5 to about 7.0, and an aqueous stabilizer solution that comprises sodium phosphate, sodium chloride, a PS80, and a BA; or
   (9) a stable aqueous pharmaceutical formulation comprising a CD123×CD3 diabody, a sodium phosphate buffer, sodium chloride, and PS80, wherein said formulation has a pH of about 6.0, and an aqueous stabilizer solution that comprises sodium phosphate, sodium chloride, a PS80, and a BA; or
   (10) a stable aqueous pharmaceutical formulation comprising a CD123×CD3 diabody, a sodium phosphate buffer, sodium chloride, and PS80, wherein said CD123×CD3 diabody has a concentration of about 0.01 mg/mL to about 1 mg/mL, and an aqueous stabilizer solution that comprises sodium phosphate, sodium chloride, a PS80, and a BA; or
   (11) a stable aqueous pharmaceutical formulation comprising a CD123×CD3 diabody, a sodium phosphate buffer, sodium chloride, and PS80, wherein said concentration of CD123×CD3 diabody is about 0.1 mg/mL, and an aqueous stabilizer solution that comprises sodium phosphate, sodium chloride, a PS80, and a BA; or
   (12) a stable aqueous pharmaceutical formulation comprising a CD123×CD3 diabody, a sodium phosphate buffer, sodium chloride, and PS80, wherein said CD123×CD3 diabody comprises:
      a) a first polypeptide chain having the amino acid sequence of SEQ ID NO:42; and
      b) a second polypeptide chain having the amino acid sequence of SEQ ID NO:44; and
      wherein the first and said second polypeptide chains are covalently bonded to one another by a disulfide bond, and an aqueous stabilizer solution that comprises sodium phosphate, sodium chloride, a PS80, and a BA; or
   (13) a stable aqueous pharmaceutical formulation comprising a CD123×CD3 diabody, a sodium phosphate buffer, sodium chloride, and PS80, wherein said formulation comprises about 0.1 mg/mL of said CD123×CD3 diabody, about 10 mM sodium phosphate, about 150 mM sodium chloride, and about 0.1 mg/mL PS80, and wherein the pH of said formulation is about 6.0, and an aqueous stabilizer solution that comprises sodium phosphate, sodium chloride, a PS80, and a BA;
   (14) a stable aqueous pharmaceutical formulation comprising a CD123×CD3 diabody, a sodium phosphate buffer, sodium chloride, and PS80, wherein said solution maintains monomeric purity of said CD123×CD3 diabody for about 3 months at 25° C., and an aqueous stabilizer solution that comprises sodium phosphate, sodium chloride, a PS80, and a BA; or
   (15) a stable aqueous pharmaceutical formulation comprising a CD123×CD3 diabody, a sodium phosphate buffer, sodium chloride, and PS80, wherein said solution maintains monomeric purity of said CD123×CD3 diabody for about 48 months at 2-8° C., and an aqueous stabilizer solution that comprises sodium phosphate, sodium chloride, a PS80, and a BA; or
(C) (1) a stable aqueous pharmaceutical formulation comprising a CD123×CD3 diabody, a sodium phosphate buffer, sodium chloride, and PS80, and an aqueous stabilizer solution that comprises sodium chloride and a PS80; or
   (2) a stable aqueous pharmaceutical formulation comprising a CD123×CD3 diabody, a sodium phosphate buffer, sodium chloride, and PS80, wherein said sodium phosphate has a concentration of about 5 mM to about 30 mM, and an aqueous stabilizer solution that comprises sodium chloride and a PS80; or
   (3) a stable aqueous pharmaceutical formulation comprising a CD123×CD3 diabody, a sodium phosphate buffer, sodium chloride, and PS80, wherein said sodium phosphate has a concentration of about 10 mM, and an aqueous stabilizer solution that comprises sodium chloride and a PS80; or
   (4) a stable aqueous pharmaceutical formulation comprising a CD123×CD3 diabody, a sodium phosphate buffer, sodium chloride, and PS80, wherein said PS80 has a concentration of about 0.05 mg/mL to about 0.3 mg/mL, and an aqueous stabilizer solution that comprises sodium chloride and a PS80; or
   (5) a stable aqueous pharmaceutical formulation comprising a CD123×CD3 diabody, a sodium phosphate buffer, sodium chloride, and PS80, wherein said PS80 has a concentration of about 0.1 mg/mL, and an aqueous stabilizer solution that comprises sodium chloride and a PS80; or
   (6) a stable aqueous pharmaceutical formulation comprising a CD123×CD3 diabody, a sodium phosphate buffer, sodium chloride, and PS80, wherein said sodium chloride has a concentration of about 100 mM to about 300 mM, and an aqueous stabilizer solution that comprises sodium chloride and a PS80; or
   (7) a stable aqueous pharmaceutical formulation comprising a CD123×CD3 diabody, a sodium phosphate buffer, sodium chloride, and PS80, wherein said sodium chloride has a concentration of about 150 mM, and an aqueous stabilizer solution that comprises sodium chloride and a PS80; or
   (8) a stable aqueous pharmaceutical formulation comprising a CD123×CD3 diabody, a sodium phosphate buffer, sodium chloride, and PS80, wherein said formulation has a pH of about 5.5 to about 7.0, and an aqueous stabilizer solution that comprises sodium chloride and a PS80; or
   (9) a stable aqueous pharmaceutical formulation comprising a CD123×CD3 diabody, a sodium phosphate buffer, sodium chloride, and PS80, wherein said formulation has a pH of about 6.0, and an aqueous stabilizer solution that comprises sodium chloride and a PS80; or
   (10) a stable aqueous pharmaceutical formulation comprising a CD123×CD3 diabody, a sodium phosphate buffer, sodium chloride, and PS80, wherein said CD123×CD3 diabody has a concentration of about 0.01 mg/mL to about 1 mg/mL, and an aqueous stabilizer solution that comprises sodium chloride and a PS80; or
   (11) a stable aqueous pharmaceutical formulation comprising a CD123×CD3 diabody, a sodium phosphate buffer, sodium chloride, and PS80, wherein said concentration of CD123×CD3 diabody is about 0.1 mg/mL, and an aqueous stabilizer solution that comprises sodium chloride and a PS80; or
   (12) a stable aqueous pharmaceutical formulation comprising a CD123×CD3 diabody, a sodium phosphate buffer, sodium chloride, and PS80, wherein said CD123×CD3 diabody comprises:
      a) a first polypeptide chain having the amino acid sequence of SEQ ID NO:42; and
      b) a second polypeptide chain having the amino acid sequence of SEQ ID NO:44; and
      wherein the first and said second polypeptide chains are covalently bonded to one another by a disulfide bond, and an aqueous stabilizer solution that comprises sodium chloride and a PS80; or
   (13) a stable aqueous pharmaceutical formulation comprising a CD123×CD3 diabody, a sodium phosphate buffer, sodium chloride, and PS80, wherein said formulation comprises about 0.1 mg/mL of said CD123×CD3 diabody, about 10 mM sodium phosphate, about 150 mM sodium chloride, and about 0.1 mg/mL PS80, and wherein the pH of said formulation is about 6.0, and an aqueous stabilizer solution that comprises sodium chloride and a PS80;
   (14) a stable aqueous pharmaceutical formulation comprising a CD123×CD3 diabody, a sodium phosphate buffer, sodium chloride, and PS80, wherein said solution maintains monomeric purity of said CD123×CD3 diabody for about 3 months at 25° C., and an aqueous stabilizer solution that comprises sodium chloride and a PS80; or
   (15) a stable aqueous pharmaceutical formulation comprising a CD123×CD3 diabody, a sodium phosphate buffer, sodium chloride, and PS80, wherein said solution maintains monomeric purity of said CD123×CD3 diabody for about 48 months at 2-8° C., and an aqueous stabilizer solution that comprises sodium chloride and a PS80.

The invention additionally provides a method of treating a hematologic malignancy using any of the above-described kits.

The invention additionally pertains to the use of the above-described dosing solutions for the treatment of a hematologic malignancy.

The invention additionally pertains to the use of the above-described kits for the treatment of a hematologic malignancy.

The invention additionally pertains to the embodiment wherein such hematologic malignancy is selected from the group consisting of: AML, CML, including blastic crisis of CML and Abelson oncogene associated with CML (Bcr-ABL translocation, MDS, B-ALL, T-ALL, CLL, including Richter's syndrome or Richter's transformation of CLL, HCL, blastic plasmacytoid dendritic cell neoplasm (BPDCN), NHL, including mantle cell lymphoma (MCL) and small lymphocytic lymphoma (SLL), Hodgkin's lymphoma, systemic mastocytosis, and Burkitt's lymphoma.

The invention particularly pertains to the embodiment wherein such hematologic malignancy is AML, BPDCN, MDS, or T-ALL.

The invention particularly pertains to the embodiment wherein the subject is a human subject.

### BRIEF DESCRIPTION OF THE DRAWINGS

FIG. 1 illustrates the overall structure of the first and second polypeptide chains of a two chain covalently bonded diabody (e.g., the CD123×CD3 bispecific diabody, DART-A) having two epitope-binding sites composed of two polypeptide chains, each having an E-coil or K-coil Heterodimer-Promoting Domain (alternative Heterodimer-Promoting Domains are provided below). A cysteine residue may be present in a linker as shown and/or in the Heterodimer-Promoting Domain (see, FIGS. 3-4). VL and VH Domains that recognize the same epitope are shown using the same shading or fill pattern. The wavy line (WWW) in this, and all of the Figures providing schematic presentations of binding molecule domains, represents one or more optional Heterodimer-Promoting Domains, that is/are preferably present.

FIG. 2 provides a schematic of a representative covalently bonded diabody molecule having two epitope-binding sites composed of two polypeptide chains, each having a CH2 and CH3 Domain, such that the associated chains form all or part of an Fc Region. VL and VH Domains that recognize the same epitope are shown using the same shading or fill pattern.

FIG. 3 provides schematics showing a representative covalently bonded tetravalent diabody having four epitope-binding sites composed of two pairs of polypeptide chains (i.e., four polypeptide chains in all). A cysteine residue is shown in the Heterodimer-Promoting domain with an optional cysteine residue present in the linker. One polypeptide chain of each pair possesses a CH2 and CH3 Domain, such that the associated chains form all or part of an Fc Region. VL and VH Domains that recognize the same epitope are shown using the same shading or fill pattern. The two pairs of polypeptide chains may be the same. In such embodiments, wherein the two pairs of polypeptide chains are the same and the VL and VH Domains recognize different epitopes, the resulting molecule possesses four epitope-binding sites and is bispecific and bivalent with respect to each bound epitope.

FIG. 4 provides schematics of a representative covalently bonded diabody molecule having two epitope-binding sites composed of three polypeptide chains. Two of the polypeptide chains possess a CH2 and CH3 Domain, such that the associated chains form all or part of an Fc Region. A cysteine residue may be present in the Heterodimer-Promoting Domain (as shown) and/or in the linker (see, FIGS. 1 and 3). The polypeptide chains comprising the VL and VH Domain further comprise a Heterodimer-Promoting Domain. VL and VH Domains that recognize the same epitope are shown using the same shading or fill pattern.

FIGS. 5A-5B show DART-A turbidity (FIG. 5A) and sub-visible particle count (FIG. 5B) after continuous agitation in two phosphate buffered saline (PBS) formulations with PS80 and two PBS formulations without PS80. The DART-A samples were held at 2-8° C. and agitated at 600 rpm over 24 hours.

FIG. 6 shows the thermal stability of DART-A. Differential scanning calorimetry (DSC) was used to test the stability of DART-A in two drug product formulations: without PS80 (DART-A DP buffer alone) or with PS80 (DART-A DP buffer+PS80). Both the control and the PS80-containing DP formulations contained 150 mM NaCl, 10 mM sodium phosphate, at pH 6.0. DSC was performed at a melting temperature (Tm) of 64° C.

FIG. 7 shows the peptide mapping results comparing peptides and modifications at the amino acid level for aged DART-A DP formulation (top panel) after 24 months storage at 5±3° C. compared to reference standard (bottom panel). The figure displays the tryptic peptide mapping results of the two samples using liquid chromatography coupled with electrospray ionization mass spectrometry (LC-ESIMS).

FIGS. 8A-8D provide the relative potency of DART-A as shown by binding to CD123 (FIG. 8A) or CD3 (FIG. 8B) after storing the DART-A DP formulation for 48 months at 5±3° C. FIG. 8C shows the percentage of DART-A monomers, as measured by SE-HPLC, following storage of the DART-A DP formulation for 48 months at 5±3° C. FIG. 8D shows the percent purity of the DART-A, as measured by CE-SDS, after storage of the DART-A DP formulation for 48 months at 5±3° C.

FIG. 9 provides a summary of the development of stabilizers for DART-A. Stabilizer 1 was developed to be mixed with the DART-A DP formulation to diminish protein loss due to absorption during administration using a dual ambulatory pump. Studies were performed to compare Stabilizer 1 (containing rHA) with a modified Stabilizer 1 (containing only PS80). Alternative preservatives were tested and these studies resulted in the development of Stabilizer 2 for single ambulatory pump administration.

FIG. 10 provides a summary of studies performed for the development of Stabilizer 2 for single pump administration. The selection of preservatives was evaluated in a series of steps as shown.

FIG. 11 shows the dual-pump ambulatory infusion configuration for continuous administration of DART-A diluted in Stabilizer 1. Sampling points (points #1-#5) in the ambulatory infusion configuration studies are shown.

FIGS. 12A-12B show the preparation of DART-A dosing solutions to be loaded onto medication cassettes by dilution of the DART-A DP formulation in Stabilizer 1. FIG. 12A shows the preparation of a DART-A high concentration (5000 ng/mL) dosing solution. FIG. 12B shows the preparation of a DART-A low concentration (100 ng/mL) dosing solution.

FIG. 13 shows the preparation of the dosing solution for single ambulatory pump administration. Stabilizer 2 (40 ml) was added to a 250 ml saline bag containing 270 ml nominal volume (alternatively/optionally a saline containing ambulatory pump cassette can be utilized in place of the saline bag). The saline bag was mixed well prior to adding the desired volume of the aqueous pharmaceutical formulation containing the DART-A. The saline bag was mixed well resulting in the final dosing solution. DART-A and preservative stability were monitored during room temperature storage for 72 hours.

FIG. 14 shows a CADD-Legacy 1 ambulatory infusion pump (Deltec, St, Paul, Minn.) that may be utilized for administration of the dosing solution, comprising the DART-A DP formulation and Stabilizer 2, to a patient.

### DETAILED DESCRIPTION OF THE INVENTION

The present invention is directed to stable aqueous pharmaceutical formulations that comprise a bispecific diabody (Diabody formulation), and aqueous stabilizer solutions for stabilizing and administering said diabody. The invention particularly concerns such DART-A DP formulations that comprise a sequence-optimized CD123×CD3 bi-specific monovalent diabody, DART-A, that is capable of simultaneously binding to CD123 and CD3. The invention further concerns the use of such DART-A DP formulations and stabilizers in the treatment of hematologic malignancies such as AML or MDS in patients.

I. Bispecific Diabodies

The present invention relates to formulations comprising bispecific diabodies, particularly covalently bonded diabodies comprising two, three or four polypeptide chains. As provided below, such covalently bonded diabody may further comprise an Fc Domain.

Stable, covalently bonded heterodimeric non-mono-specific diabodies, termed DART® diabodies, have been described (see, e.g., PCT Publication Nos. WO 2006/113665; WO 2008/157379; WO 2010/027797; WO 2010/033279; WO 2010/080538; WO 2011/109400; WO 2012/018687; WO 2012/162067; WO 2012/162068; WO 2014/159940; WO 2015/021089; WO 2015/026892; and WO 2015/026894). Such covalently bonded diabodies comprises two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody's binding characteristics.

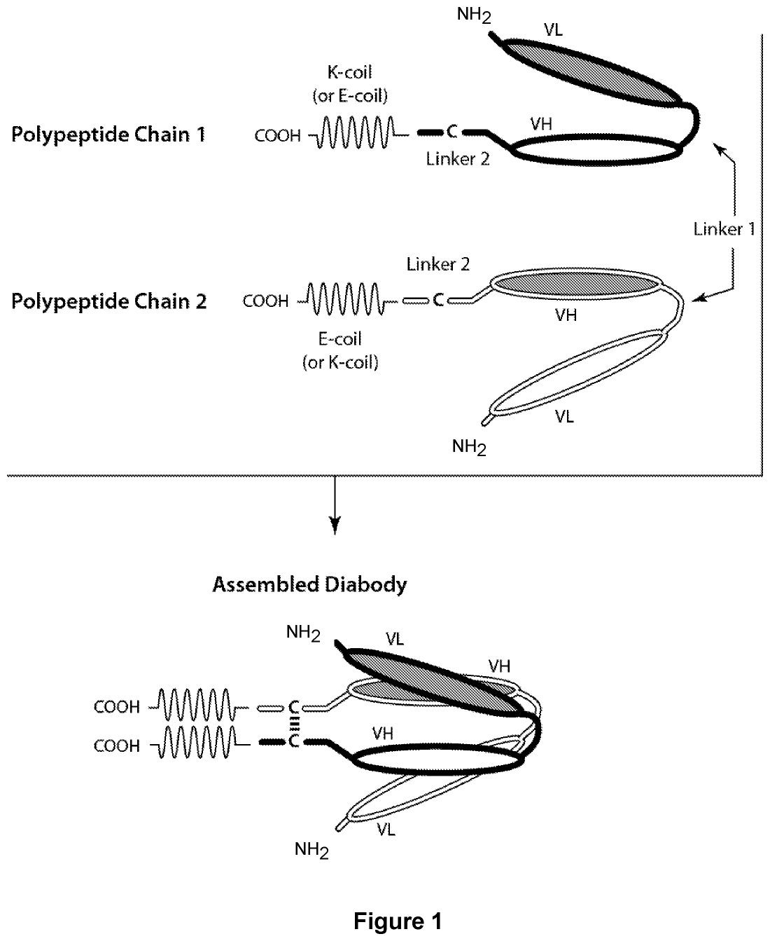

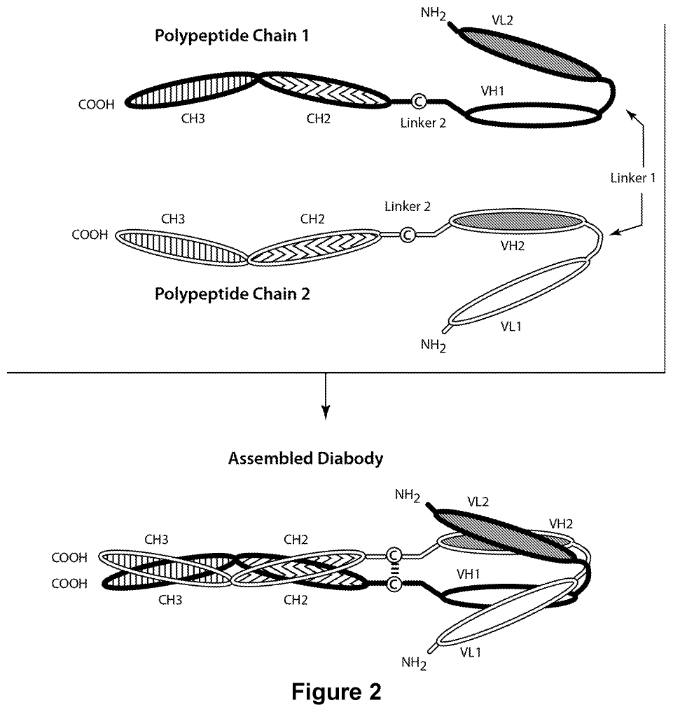

The simplest covalently bonded diabody comprises two polypeptide chains each comprising three Domains (FIG. 1). The first polypeptide chain comprises: (i) a Domain that comprises a binding region of a light chain variable Domain of the a first immunoglobulin (VL1), (ii) a second Domain that comprises a binding region of a heavy chain variable Domain of a second immunoglobulin (VH2), and (iii) a third Domain that serves to promote heterodimerization (a “Heterodimer-Promoting Domain”) with the second polypeptide chain and to covalently bond the first polypeptide to the second polypeptide chain of the diabody. The second polypeptide chain contains a complementary first Domain (a VL2 Domain), a complementary second Domain (a VH1 Domain) and a third Domain that complexes with the third Domain of the first polypeptide chain in order to promote heterodimerization (a “Heterodimer-Promoting Domain”) and covalent bonding with the first polypeptide chain. Such molecules are stable, potent and have the ability to simultaneously bind two or more antigens. In one embodiment, the third Domains of the first and second polypeptide chains each contain a cysteine (“C”) residue, which serves to bind the polypeptides together via a disulfide bond. The third Domain of one or both of the polypeptide chains may additionally possess the sequence of a CH2-CH3 Domain, such that complexing of the diabody polypeptides forms an Fc Domain that is capable of binding to the Fc receptor of cells (such as B lymphocytes, dendritic cells, natural killer cells, macrophages, neutrophils, eosinophils, basophils and mast cells) (see, e.g., FIGS. 2-4). Several examples of such covalently bonded molecules are described in detail below and many variations of such molecules have been described elsewhere (see, e.g., United States Patent Publication Nos. 2013-0295121; 2010-0174053; 2007-0004909; 2009-0060910; European Patent Publication No. EP 2714079; EP 2601216; EP 2376109; EP 2158221 and PCT Publication Nos. WO 2012/162068; WO 2012/018687; WO 2010/080538; WO 2006/113665).

A. Covalently Bonded Diabodies Lacking Fc Domains

The first polypeptide chain of a covalently bonded diabody lacking an Fc preferably comprises, in the N-terminal to C-terminal direction: an N-terminus, the VL Domain of a monoclonal antibody capable of binding either the first or second epitope (i.e., either VL1 or VL2), a first intervening spacer peptide (Linker 1), a VH Domain of a monoclonal antibody capable of binding the second epitope (if such first polypeptide chain contains VL1) or a VH Domain of a monoclonal antibody capable of binding the first epitope (if such first polypeptide chain contains VL2), a second intervening spacer peptide (Linker 2) optionally containing a cysteine residue, a Heterodimer-Promoting Domain and a C-terminus (FIG. 1).

The second polypeptide chain comprises, in the N-terminal to C-terminal direction: an N-terminus, the VL Domain of a monoclonal antibody capable of binding the first or second epitope (i.e., VL1 or VL2, and being the VL Domain not selected for inclusion in the first polypeptide chain of the diabody), an intervening spacer peptide (Linker 1), a VH Domain of a monoclonal antibody capable of binding either the first or second epitope (i.e., VH1 or VH2, and being the VH Domain not selected for inclusion in the first polypeptide chain of the diabody), a second intervening spacer peptide (Linker 2) optionally containing a cysteine residue, a Heterodimer-Promoting Domain and a C-terminus (FIG. 1). The employed VL and VH Domains specific for a particular epitope are preferably obtained or derived from the same monoclonal antibody. However, such domains may be derived from different monoclonal antibodies provided that they associate to form a functional Binding Domain capable of immunospecifically binding such epitope. Such different antibodies are referred to herein as being “corresponding” antibodies.

The VL Domain of the first polypeptide chain interacts with the VH Domain of the second polypeptide chain to form a first functional epitope binding domain that is specific for one of the epitopes (e.g., the first epitope). Likewise, the VL Domain of the second polypeptide chain interacts with the VH Domain of the first polypeptide chain in order to form a second functional epitope binding domain that is specific for the other epitope (i.e., the second epitope). Thus, the selection of the VL and VH Domains of the first and second polypeptide chains is “coordinated,” such that the two polypeptide chains of the diabody collectively comprise VL and VH Domains capable of binding both the first epitope and the second epitope (i.e., they collectively comprise VL1/VH1 and VL2/VH2).

Most preferably, the length of the intervening spacer peptide (i.e., “Linker 1,” which separates such VL and VH Domains) is selected to substantially or completely prevent the VL and VH Domains of the polypeptide chain from binding one another (for example consisting of from 0, 1, 2, 3, 4, 5, 6, 7, 8 or 9 intervening linker amino acid residues). Thus, the VL and VH Domains of the first polypeptide chain are substantially or completely incapable of binding one another. Likewise, the VL and VH Domains of the second polypeptide chain are substantially or completely incapable of binding one another. A preferred intervening spacer peptide (Linker 1) has the sequence (SEQ ID NO:1): GGGSGGGG.

The length and composition of the second intervening spacer peptide (“Linker 2”) is selected based on the choice of one or more polypeptide domains that promote such dimerization (i.e., a “Heterodimer-Promoting Domain”). Typically, the second intervening spacer peptide (Linker 2) will comprise 3-20 amino acid residues. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more cysteines. A preferred cysteine-containing spacer peptide (Linker 2) has the sequence GGCGGG (SEQ ID NO:2). Alternatively, Linker 2 does not comprise a cysteine (e.g., ASTKG (SEQ ID NO:3)) and a cysteine-containing Heterodimer-Promoting Domain, as described below is used. Optionally, both a cysteine-containing Linker 2 and a cysteine-containing Heterodimer-Promoting Domain are used.

Exemplary Heterodimer-Promoting Domains include for example, those comprising polypeptide coils of opposing charge. Preferred Heterodimer-Promoting Domains will comprise an “E-coil” Heterodimer-Promoting Domain (SEQ ID NO:4: [symbol] VAAL [symbol] K- [symbol] VAAL [symbol] K- [symbol] VAAL [symbol] K- [symbol] VAAL [symbol] K), whose glutamate residues will form a negative charge at pH 7, or a “K-coil” Heterodimer-Promoting Domain (SEQ ID NO:5: [symbol] VAAL [symbol] E- [symbol] VAAL [symbol] E- [symbol] VAAL [symbol] E- [symbol] VAAL [symbol] E), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence [symbol] VAA [symbol] K- [symbol] VAAL [symbol] K- [symbol] VAAL [symbol] K- [symbol] VAAL [symbol] K (SEQ ID NO:6), and a modified K-coil having the amino acid sequence [symbol] VAA [symbol] E- [symbol] VAAL [symbol] E- [symbol] VAAL [symbol] E- [symbol] VAAL [symbol] E (SEQ ID NO:7).

B. Bispecific Diabodies Comprising Fc Domains

The addition of an IgG CH2-CH3 Domain to one or both of the diabody polypeptide chains, such that the complexing of the diabody chains results in the formation of an Fc Domain, increases the biological half-life and/or alters the valency of the diabody. Such diabodies comprise, two or more polypeptide chains whose sequences permit the polypeptide chains to covalently bind each other to form a covalently associated diabody that is capable of simultaneously binding the First Epitope and the Second Epitope. Incorporating an IgG CH2-CH3 Domains onto both of the diabody polypeptides will permit a two-chain bispecific Fc Domain-containing diabody to form (FIG. 2).

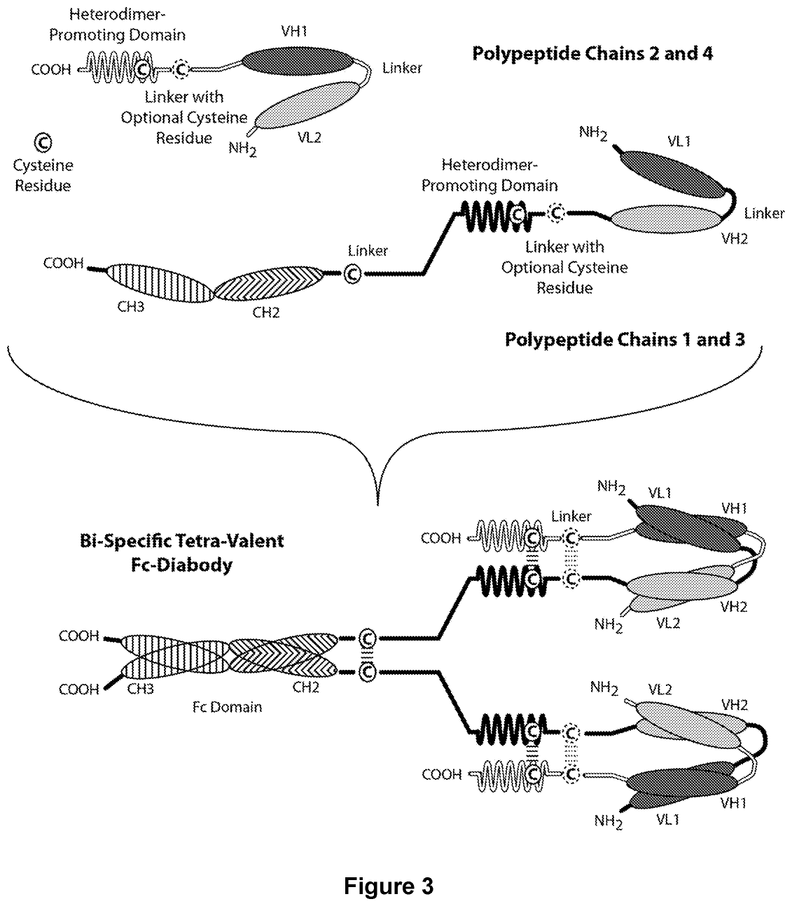

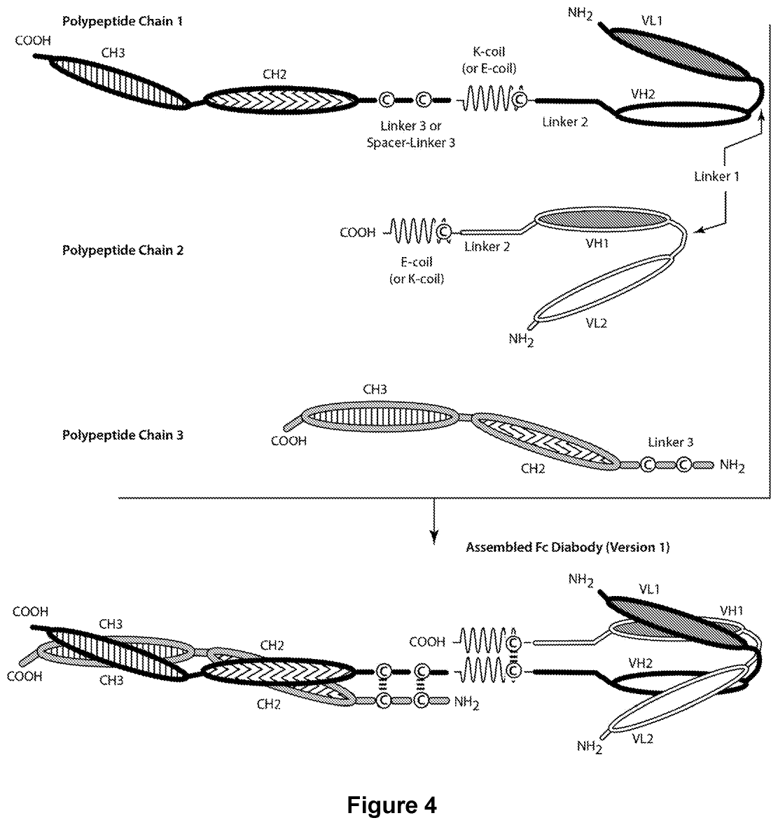

Alternatively, incorporating IgG CH2-CH3 Domains onto only one of the diabody polypeptides will permit a more complex four-chain bispecific Fc Domain-containing diabody to form (FIG. 3). The addition of a third polypeptide chain comprising a second CH2-CH3 Fc Domain, but lacking any binding domains permits the formation of three-chain bispecific Fc Domain-containing diabody (FIG. 4).

The CH2-CH3 Domains of such molecules may be of any isotype (e.g., IgG1, IgG2, IgG3, or IgG4). The molecules may further comprise a Hinge Domain. When present, the Hinge Domain may be of any isotype (e.g., IgG1, IgG2, IgG3, or IgG4), and is preferably of the same isotype as the desired Fc Domain. The CH2-CH3 Domains may further comprise one or more amino acid substitutions to reduce or eliminate binding to Fc-receptor(s) (see, e.g., U.S. Pat. No. 5,624,821), and/or to enhance serum half-life (see, e.g., U.S. Pat. No. 7,083,784), and/or to foster heterodimerization between the CH2-CH3 Domains present on two different polypeptide chains (see, e.g., U.S. Pat. Nos. 5,731,168 and 7,183,076).

One exemplary IgG1 sequence for the CH2 and CH3 Domains having reduced or abolished effector function will comprise the substitutions L234A/L235A (SEQ ID NO:8):

APE [symbol] GGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPG

[symbol]

wherein [symbol] is lysine (K) or is absent.

One exemplary IgG1 sequence for the CH2 and CH3 Domains having increased serum half-life may combine the reduced or abolished effector function provided by the substitutions L234A/L235A and the increased serum half-life provided by the substitutions M252Y/S254T/T256E (SEQ ID NO:9):

APE [symbol] GGPSV FLFPPKPKDT L [symbol] I [symbol] R [symbol] PEVT
CVVVDVSHED PEVKFNWYVD GVEVHNAKTK
PREEQYNSTY RVVSVLTVLH QDWLNGKEYK
CKVSNKALPA PIEKTISKAK GQPREPQVYT
LPPSREEMTK NQVSLTCLVK GFYPSDIAVE
WESNGQPENN YKTTPPVLDS DGSFFLYSKL
TVDKSRWQQG NVFSCSVMHE ALHNHYTQKS
LSLSPG

[symbol]

wherein [symbol] is lysine (K) or is absent.

Exemplary IgG1 amino acid sequences for the CH2 and CH3 Domains that heterodimerize will comprise a T366W (“Knob”) substitution on one chain and the T366S/L368A/Y407V (“Hole”) substitutions on the other chain. These substitutions may be combined with further substitutions which reduced or abolished effector function and/or increase serum half-life.

One exemplary IgG1 amino acid sequence for a knob-bearing CH2-CH3 Domain further combines the reduced or abolished effector function provided by the substitutions L234A/L235A (SEQ ID NO:10):

APE [symbol] GGPSV FLFPPKPKDT LMISRTPEVT
CVVVDVSHED PEVKFNWYVD GVEVHNAKTK
PREEQYNSTY RVVSVLTVLH QDWLNGKEYK
CKVSNKALPA PIEKTISKAK GQPREPQVYT
LPPSREEMTK NQVSL [symbol] CLVK GFYPSDIAVE
WESNGQPENN YKTTPPVLDS DGSFFLYSKL
TVDKSRWQQG NVFSCSVMHE ALHNHYTQKS
LSLSPG

[symbol]

wherein [symbol] is lysine (K) or is absent.

One exemplary IgG1 amino acid sequence for a hole-bearing CH2-CH3 Domain further combines the reduced or abolished effector function provided by the substitutions L234A/L235A (SEQ ID NO:11):

APE [symbol] GGPSV FLFPPKPKDT LMISRTPEVT
CVVVDVSHED PEVKFNWYVD GVEVHNAKTK
PREEQYNSTY RVVSVLTVLH QDWLNGKEYK
CKVSNKALPA PIEKTISKAK GQPREPQVYT
LPPSREEMTK NQVSL [symbol] C [symbol] VK GFYPSDIAVE
WESNGQPENN YKTTPPVLDS DGSFFL [symbol] SKL
TVDKSRWQQG NVFSCSVMHE ALHN [symbol] YTQKS
LSLSPG

[symbol]

wherein [symbol] is lysine (K) or is absent.

Fc Domain-containing diabody molecules of the present invention may include additional intervening spacer peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Domain (preferably a cysteine-containing portion of an IgG Hinge Domain possessing 1, 2, 3 or more cysteine residues). Exemplary linkers that may be employed in the bispecific Fc Domain-containing diabody molecules of the present invention include: GGGS (SEQ ID NO:12), GGCGGG (SEQ ID NO:13), ASTKG (SEQ ID NO:14), APSSS (SEQ ID NO:15), APSSSPME (SEQ ID NO:16), Additionally, the amino acids GGG, or LEPKSS (SEQ ID NO:17) may be immediately followed by DKTHTCPPCP (SEQ ID NO:18) to form the alternate linkers: GGGDKTHTCPPCP (SEQ ID NO:19); and LEPKSSDKTHTCPPCP (SEQ ID NO:20). Bispecific Fc Domain-containing molecules of the present invention may incorporate an IgG Hinge Domain in addition to or in place of a linker. Exemplary Hinge Domains include: EPKSCDKTHTCPPCP (SEQ ID NO:21) from IgG1, ERKCCVECPPCP (SEQ ID NO: 22) from IgG2, ELKTPLGDTTHTCPRCPEPKSCDTPPPCPRCPEPKSCDTPPPCPRCPEPKSCDTPPP CPRCP (SEQ ID NO:23) from IgG3, ESKYGPPCPSCP (SEQ ID NO:24) from IgG4, and ESKYGPPCPPCP (SEQ ID NO:25) an IgG4 Hinge variant comprising a stabilizing S228P substitution (as numbered by the EU index as set forth in Kabat) to reduce strand exchange.

As provided in FIG. 3, Fc Domain-containing diabodies of the invention may comprise four chains. The first and third polypeptide chains of such a diabody contain three domains: (i) a VL1-containing Domain, (ii) a VH2-containing Domain, (iii) a Heterodimer-Promoting Domain, and (iv) a Domain containing a CH2-CH3 sequence. The second and fourth polypeptide chains contain: (i) a VL2-containing Domain, (ii) a VH1-containing Domain, and (iii) a Heterodimer-Promoting Domain, where the Heterodimer-Promoting Domains promote the dimerization of the first/third polypeptide chains with the second/fourth polypeptide chains. The general structure of the polypeptide chains of a representative four-chain bispecific Fc Domain-containing diabody is provided in Table 1:

TABLE 1

| | | |
|---|---|---|
| Bispecific | 2^{nd} Chain | NH₂-VL2-VH1-HPD-COOH |
| | 1^{st} Chain | NH₂-VL1-VH2-HPD-CH2-CH3-COOH |
| | 1^{st} Chain | NH₂-VL1-VH2-HPD-CH2-CH3-COOH |
| | 2^{nd} Chain | NH₂-VL2-VH1-HPD-COOH |

HPD = Heterodimer-Promoting Domain

A provided in FIG. 4, the Fc Domain-containing diabodies of the present invention may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 Epitope Binding Domain that is capable of binding either the First or Second Epitope, as well as a VL2/VH2 Epitope Binding Domain that is capable of binding the other of such epitopes. The first and second polypeptides are bonded to one another through a disulfide bond involving cysteine residues in their respective Third Domains. Notably, the first and third polypeptide chains complex with one another to form an Fc Domain that is stabilized via a disulfide bond. The general structure of the polypeptide chains of a representative three-chain bispecific Fc Domain-containing diabody is provided in (Table 2):

TABLE 2

| | | |
|---|---|---|
| First | 3^{rd} Chain | NH₂-CH2-CH3-COOH |
| Orientation | 1^{st} Chain | NH₂-VL1-VH2-HPD-CH2-CH3-COOH |
| | 2^{nd} Chain | NH₂-VL2-VH1-HPD-COOH |

HPD = Heterodimer-Promoting Domain

### II. The Polypeptide Chains of DART-A

DART-A is a sequence-optimized bispecific diabody capable of simultaneously and specifically binding to an epitope of CD123 and to an epitope of CD3 (a “CD123×CD3” bi-specific diabody) (US Patent Publn. No. US 2016-0200827, in PCT Publn. No. WO 2015/026892, in Al-Hussaini, M. et al. (2016) “Targeting CD123 In Acute Myeloid Leukemia Using A T-Cell-Directed Dual-Affinity Retargeting Platform,” Blood 127:122-131, in Vey, N. et al. (2017) “A Phase 1, First-in-Human Study of MGD006/580880 (CD123×CD3) in AML/MDS,” 2017 ASCO Annual Meeting, Jun. 2-6, 2017, Chicago, Ill.: Abstract TPS7070, each of which documents is herein incorporated by reference in its entirety). DART-A was found to exhibit enhanced functional activity relative to other non-sequence-optimized CD123×CD3 bispecific diabodies of similar composition, and is thus termed a “sequence-optimized” CD123×CD3 bi-specific diabody. PCT Application No. PCT/US2017/050471 describes preferred dosing regimens for administering DART-A to patients, and is herein incorporated by reference in its entirety.

DART-A has the general structure shown in FIG. 1 and comprises a first polypeptide chain and a second polypeptide chain. The first polypeptide chain of the bi-specific diabody will comprise, in the N-terminal to C-terminal direction, an N-terminus, a Light Chain Variable Domain (VL Domain) of a monoclonal antibody capable of binding to CD3 (VL_{CD3}), an intervening linker peptide (Linker 1), a Heavy Chain Variable Domain (VH Domain) of a monoclonal antibody capable of binding to CD123 (VH_{CD123}), and a C-terminus. A preferred sequence for such a VL_{CD3} Domain is SEQ ID NO:26:

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG

The Antigen Binding Domain of VL_{CD3} comprises

CDR1 SEQ ID NO: 27:
RSSTGAVTTSNYAN,
CDR2 SEQ ID NO: 28:
GTNKRAP,
and
CDR3 SEQ ID NO: 29:
ALWYSNLWV.

A preferred sequence for such Linker 1 is SEQ ID NO:1: GGGSGGGG. A preferred sequence for such a VH_{CD123} Domain is SEQ ID NO:30:

EVQLVQSGAE LKKPGASVKV SCKASGYTFT
DYYMKWVRQA PGQGLEWIGD IIPSNGATFY
NQKFKGRVTI TVDKSTSTAY MELSSLRSED
TAVYYCARSH LLRASWFAYW GQGTLVTVSS

The Antigen Binding Domain of VH_{CD123} comprises

CDR1 SEQ ID NO: 31:
DYYMK,
CDR2 SEQ ID NO: 32:
DIIPSNGATFYNQKFKG,
and
CDR3 SEQ ID NO: 33:
SHLLRASFAY.

The second polypeptide chain will comprise, in the N-terminal to C-terminal direction, an N-terminus, a VL domain of a monoclonal antibody capable of binding to CD123 (VL_{CD123}), an intervening linker peptide (e.g., Linker 1), a VH domain of a monoclonal antibody capable of binding to CD3 (VH_{CD3}), and a C-terminus. A preferred sequence for such a VL_{CD123} Domain is SEQ ID NO:34:

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIK

The Antigen Binding Domain of VL_{CD123} comprises

CDR1 SEQ ID NO: 35:
KSSQSLLNSGNQKNYLT,
CDR2 SEQ ID NO: 36:
WASTRES,
and
CDR3 SEQ ID NO: 37:
QNDYSYPYT.

A preferred sequence for such a VH_{CD3} Domain is SEQ ID NO:38:

EVQLVESGGG LVQPGGSLRL SCAASGFTFS
TYAMNWVRQA PGKGLEWVGR IRSKYNNYAT
YYADSVKDRF TISRDDSKNS LYLQMNSLKT
EDTAVYYCVR HGNFGNSYVS WFAYWGQGTL
VTVSS

The Antigen Binding Domain of VH_{CD3} comprises

CDR1 SEQ ID NO: 39:
TYAMN,
CDR2 SEQ ID NO: 40:
RIRSKYNNYATYYADSVKD,
and
CDR3 SEQ ID NO: 41:
HGNFGNSYVSWFAY.

The sequence-optimized CD123×CD3 bi-specific diabodies of the present invention are engineered so that such first and second polypeptides covalently bond to one another via cysteine residues along their length. Such cysteine residues may be introduced into the intervening linker (e.g., Linker 1) that separates the VL and VH domains of the polypeptides. Alternatively, and more preferably, a second peptide (Linker 2) is introduced into each polypeptide chain, for example, at a position N-terminal to the VL domain or C-terminal to the VH domain of such polypeptide chain. A preferred sequence for such Linker 2 is SEQ ID NO:13: GGCGGG.

As provided above, formation of heterodimers can be driven by further engineering such polypeptide chains to contain polypeptide coils of opposing charge. Thus, in a preferred embodiment, one of the polypeptide chains will be engineered to contain an “E-coil” domain (SEQ ID NO:4: [symbol] VAAL [symbol] K [symbol] VAAL [symbol] K [symbol] VAAL [symbol] K [symbol] VAAL [symbol] K), while the other of the two polypeptide chains will be engineered to contain an “K-coil” domain (SEQ ID NO:5: [symbol] VAAL [symbol] E [symbol] VAAL [symbol] E [symbol] VAAL [symbol] E [symbol] VAAL [symbol] E).

It is immaterial which coil is provided to the first or second polypeptide chains. However, a preferred sequence-optimized CD123×CD3 bi-specific diabody of the present invention, DART-A, has a first polypeptide chain having the sequence (SEQ ID NO:42):

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG GGGSGGGGEV
QLVQSGAELK KPGASVKVSC KASGYTFTDY
YMKWVRQAPG QGLEWIGDII PSNGATFYNQ
KFKGRVTITV DKSTSTAYME LSSLRSEDTA
VYYCARSHLL RASWFAYWGQ GTLVTVSSGG
CGGGEVAALE KEVAALEKEV AALEKEVAAL
EK

DART-A Chain 1 is composed of: SEQ ID NO:26-SEQ ID NO:1-SEQ ID NO:30-SEQ ID NO:13-SEQ ID NO:4. A DART-A Chain 1 encoding polynucleotide is SEQ ID NO:43:

caggctgtgg tgactcagga gccttcactg
accgtgtccc caggcggaac tgtgaccctg
acatgcagat ccagcacagg cgcagtgacc
acatctaact acgccaattg ggtgcagcag
aagccaggac aggcaccaag gggcctgatc
gggggtacaa acaaaagggc tccctggacc
cctgcacggt tttctggaag tctgctgggc
ggaaaggccg ctctgactat taccggggca
caggccgagg acgaagccga ttactattgt
gctctgtggt atagcaatct gtgggtgttc
gggggtggca caaaactgac tgtgctggga
gggggtggat ccggcggcgg aggcgaggtg
cagctggtgc agtccggggc tgagctgaag
aaacccggag cttccgtgaa ggtgtcttgc
aaagccagtg gctacacctt cacagactac
tatatgaagt gggtcaggca ggctccagga
cagggactgg aatggatcgg cgatatcatt
ccttccaacg gggccacttt ctacaatcag
aagtttaaag gcagggtgac tattaccgtg
gacaaatcaa caagcactgc ttatatggag
ctgagctccc tgcgctctga agatacagcc
gtgtactatt gtgctcggtc acacctgctg
agagccagct ggtttgctta ttggggacag
ggcaccctgg tgacagtgtc ttccggagga
tgtggcggtg gagaagtggc cgcactggag
aaagaggttg ctgctttgga gaaggaggtc
gctgcacttg aaaaggaggt cgcagccctg
gagaaa

The second polypeptide chain of DART-A has the sequence (SEQ ID NO:44):

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIKGGGSGGG
GEVQLVESGG GLVQPGGSLR LSCAASGFTF
STYAMNWVRQ APGKGLEWVG RIRSKYNNYA
TYYADSVKDR FTISRDDSKN SLYLQMNSLK
TEDTAVYYCV RHGNFGNSYV SWFAYWGQGT
LVTVSSGGCG GGKVAALKEK VAALKEKVAA
LKEKVAALKE

DART-A Chain 2 is composed of: SEQ ID NO:34-SEQ ID NO:1-SEQ ID NO:38-SEQ ID NO:13-SEQ ID NO:5. A DART-A Chain 2 encoding polynucleotide is SEQ ID NO:45:

gacttcgtga tgacacagtc tcctgatagt
ctggccgtga gtctggggga gcgggtgact
atgtcttgca agagctccca gtcactgctg
aacagcggaa atcagaaaaa ctatctgacc
tggtaccagc agaagccagg ccagccccct
aaactgctga tctattgggc ttccaccagg
gaatctggcg tgcccgacag attcagcggc
agcggcagcg gcacagattt taccctgaca
atttctagtc tgcaggccga ggacgtggct
gtgtactatt gtcagaatga ttacagctat
ccctacactt tcggccaggg gaccaagctg
gaaattaaag gaggcggatc cggcggcgga
ggcgaggtgc agctggtgga gtctggggga
ggcttggtcc agcctggagg gtccctgaga
ctctcctgtg cagcctctgg attcaccttc
agcacatacg ctatgaattg ggtccgccag
gctccaggga aggggctgga gtgggttgga
aggatcaggt ccaagtacaa caattatgca
acctactatg ccgactctgt gaaggataga
ttcaccatct caagagatga ttcaaagaac
tcactgtatc tgcaaatgaa cagcctgaaa
accgaggaca cggccgtgta ttactgtgtg
agacacggta acttcggcaa ttcttacgtg
tcttggtttg cttattgggg acaggggaca
ctggtgactg tgtcttccgg aggatgtggc
ggtggaaaag tggccgcact gaaggagaaa
gttgctgctt tgaaagagaa ggtcgccgca
cttaaggaaa aggtcgcagc cctgaaagag

### III. The Properties of DART-A

DART-A was found to have the ability to simultaneously bind CD123 and CD3 as assayed using human and cynomolgus monkey cells (Table 3). Provision of DART-A was found to cause T cell activation, to mediate blast reduction, to drive T cell expansion, to induce T cell activation, and to cause the redirected killing of target cancer cells.

TABLE 3
Equilibrium Dissociation Constants (K_{D}) for the Binding of DART-A to Human
and Cynomolgus Monkey CD3 and CD123

| | kₐ (± SD) | k_{d} (± SD) | K_{D} (± SD) |
|---|---|---|---|
| Antigens | (M⁻¹s⁻¹) | (s⁻¹) | (nM) |
| Human CD3ε/δ | 5.7 (± 0.6) × 10⁵ | 5.0 (± 0.9) × 10⁻³ | 9.0 ± 2.3 |
| Cynomolgus CD3ε/δ | 5.5 (± 0.5) × 10⁵ | 5.0 (± 0.9) × 10⁻³ | 9.2 ± 2.3 |
| Human CD123-His | 1.6 (± 0.4) × 10⁶ | 1.9 (± 0.4) × 10⁻⁴ | 0.13 ± 0.01 |
| Cynomolgus CD123-His | 1.5 (± 0.3) × 10⁶ | 4.0 (± 0.7) × 10⁻⁴ | 0.27 ± 0.02 |

More particularly, DART-A was found to exhibit a potent redirected killing ability with concentrations required to achieve 50% of maximal activity (EC50s) in sub-ng/mL range, regardless of CD3 epitope binding specificity in target cell lines with high CD123 expression (Kasumi-3 (EC₅₀=0.01 ng/mL)) medium CD123-expression (Molm13 (EC₅₀=0.18 ng/mL) and THP-1 (EC₅₀=0.24 ng/mL)) and medium low or low CD123 expression (TF-1 (EC₅₀=0.46 ng/mL) and RS4-11 (EC₅₀=0.5 ng/mL)). Similarly, DART-A-redirected killing was also observed with multiple target cell lines with T cells from different donors and no redirected killing activity was observed in cell lines that do not express CD123. Results are summarized in Table 4.

TABLE 4

| | | EC₅₀ of Sequence- | |
|---|---|---|---|
| | CD123 Surface | Optimized CD123 × | |
| | Expression | CD3 Bi-Specific | |
| Target Cell | (Antibody | Diabodies (ng/mL) | |
| Line | Binding Sites) | E:T = 10:1 | Max % Killing |
| Kasumi-3 | 118620 | 0.01 | 94 |
| Molm13 | 27311 | 0.18 | 43 |
| THP-1 | 58316 | 0.24 | 40 |
| TF-1 | 14163 | 0.46 | 46 |
| RS4-11 | 957 | 0.5 | 60 |
| A498 | Negative | No activity | No activity |
| HT29 | Negative | No activity | No activity |

Additionally, when human T cells and tumor cells (Molm13 or RS4-11) were combined and injected subcutaneously into NOD/SCID gamma (NSG) knockout mice, the MOLM13 tumors was significantly inhibited at the 0.16, 0.5, 0.2, 0.1, 0.02, and 0.004 mg/kg dose levels. A dose of 0.004 mg/kg and higher was active in the MOLM13 model. The lower DART-A doses associated with the inhibition of tumor growth in the MOLM13 model compared with the RS4-11 model are consistent with the in vitro data demonstrating that MOLM13 cells have a higher level of CD123 expression than RS4-11 cells, which correlated with increased sensitivity to DART-A-mediated cytotoxicity in vitro in MOLM13 cells.

DART-A was found to be active against primary AML specimens (bone marrow mononucleocytes (BMNC) and peripheral blood mononucleocytes (PBMC)) from AML patients. Incubation of primary AML bone marrow samples with DART-A resulted in depletion of the leukemic cell population over time, accompanied by a concomitant expansion of the residual T cells (both CD4 and CD8) and the induction of T cell activation markers (CD25 and Ki-67). Upregulation of granzyme B and perforin levels in both CD8 and CD4 T cells was observed. Incubation of primary ALL bone marrow samples with DART-A resulted in depletion of the leukemic cell population over time compared to untreated control or Control DART. When the T cells were counted (CD8 and CD4 staining) and activation (CD25 staining) were assayed, the T cells expanded and were activated in the DART-A sample compared to untreated or Control DART samples. DART-A was also found to be capable of mediating the depletion of pDCs cells in both human and cynomolgus monkey PBMCs, with cynomolgus monkey pDCs being depleted as early as 4 days post infusion with as little as 10 ng/kg DART-A. No elevation in the levels of cytokines interferon-gamma, TNF-alpha, IL-6, IL-5, IL-4 and IL-2 were observed in DART-A-treated animals. These data indicate that DART-A-mediated target cell killing was mediated through a granzyme B and perforin pathway.

No activity was observed against CD123-negative targets (U937 cells) or with Control DART, indicating that the observed T cell activation was strictly dependent upon target cell engagement and that monovalent engagement of CD3 by DART-A was insufficient to trigger T cell activation.

In sum, DART-A is an antibody-based molecule engaging the CD3c subunit of the TCR to redirect T lymphocytes against cells expressing CD123, an antigen up-regulated in several hematologic malignancies. DART-A binds to both human and cynomolgus monkey antigens with similar affinities and redirects T cells from both species to kill CD123+ cells. Monkeys infused 4 or 7 days a week with weekly escalating doses of DART-A showed depletion of circulating CD123+ cells 72 h after treatment initiation that persisted throughout the 4 weeks of treatment, irrespective of dosing schedules. A decrease in circulating T cells also occurred, but recovered to baseline before the subsequent infusion in monkeys on the 4-day dose schedule, consistent with DART-A-mediated mobilization. DART-A administration increased circulating PD1+, but not TIM-3+, T cells; furthermore, ex vivo analysis of T cells from treated monkeys exhibited unaltered redirected target cell lysis, indicating no exhaustion. Toxicity was limited to a minimal transient release of cytokines following the DART-A first infusion, but not after subsequent administrations even when the dose was escalated, and a minimal reversible decrease in red cell mass with concomitant reduction in CD123+bone marrow progenitors.

### IV. Pharmaceutical Formulations

The stable aqueous pharmaceutical formulations (e.g., DART-A DP formulations) of the invention comprise a covalently bonded diabody comprising two, three, or four polypeptide chains (e.g., DART-A) and optionally, a pharmaceutically acceptable carrier, to be used with an aqueous stabilizer.

As used herein, the term “pharmaceutically acceptable carrier” is intended to refer to a diluent, adjuvant (e.g., Freund's adjuvant (complete and incomplete)), excipient, or vehicle that is approved by a regulatory agency or listed in the U.S. Pharmacopeia or in another generally recognized pharmacopeia as being suitable for delivery into animals, and more particularly, humans. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

As used herein, the term “about” refers to a standard deviation of +10%.

As used herein, the term “aqueous” refers to a water containing solution.

As used herein, the term “stable” refers to monomeric purity of a diabody in a pharmaceutical formulation or in a dosing solution, wherein said loss of monomeric purity is less than about 20%, or more preferably, a loss of less than about 15%, or more preferably, a loss of less than about 10%, or more preferably, a loss of less than about 5%, or more preferably, a loss of less than about 4%, or more preferably, a loss of less than about 3%, or more preferably, a loss of less than about 2%, or more preferably, a loss of less than about 1%, or more preferably, a loss of less than about 0.6%, or more preferably, a loss of less than about 0.4%, or more preferably, a loss of less than about 0.2% of monomeric diabody (e.g., DART-A), wherein the HMW and/or LMW species of the diabody (e.g., DART-A) in the formulation is measured via SE-HPLC.

Monomeric purity of a diabody in a pharmaceutical formulation is maintained for at least about 1 month 25° C., at least about 2 months at 25° C., at least about 3 months at about 25° C., at least about 6 months at 2-8° C., at least about 12 months at 2-8° C., at least about 18 months at 2-8° C., at least about 24 months at 2-8° C., at least about 36 months at 2-8° C., or at least about 48 months at 2-8° C. Preferably, monomeric purity of the diabody in a pharmaceutical formulation is maintained at least about 3 months at 25° C. More preferably, monomeric purity of the diabody in a pharmaceutical formulation is maintained at least about 48 months at 2-8° C.

A. Preferred Aqueous Stabilizers

The present invention particularly pertains to aqueous stabilizer solutions that will act to maintain the monomeric purity of a diabody, particularly a covalently bonded diabody having two, three, or four polypeptide chains (e.g., DART-A), act to maintain protein stability, act to diminish or prevent non-specific adsorption of the diabody (e.g., DART-A) to the surface of a container, and/or act to diminish or prevent microbial growth in the pharmaceutical formulation during storage.

As used herein, the term “aqueous stabilizer solution” denotes a water-containing solution that:
(1) acts to maintain the monomeric purity of the diabody of the formulation (e.g., DART-A DP formulation) (i.e., acts to inhibit the extent of multimerization of a diabody, such as DART-A aggregation relative to that which would be observed in the absence of such aqueous stabilizer); or
(2) acts to maintain protein stability (i.e., acts to inhibit the formation of sub-visible particles, such as low molecular weight fragments of a diabody, such as DART-A, or high molecular weight aggregates of a diabody, such as DART-A, relative to that or those that would be observed in the absence of such aqueous stabilizer) or
(3) acts to inhibit or prevent non-specific adsorption of the diabody of the formulation (e.g., DART-A DP formulation) to the surface of a container (e.g., an IV bag, IV tubing, etc.); or
(4) acts to inhibit or prevent microbial growth (such as the growth of Pseudomonas aeruginosa, Staphylococcus aureus, Escherichia coli, Candida albicans, Aspergillus brasiliensis, etc.) within the diabody formulation (e.g., DART-A DP formulation) during its storage.

As used herein, an aqueous stabilizer solution is said to act to maintain the monomeric purity of a diabody if its presence causes a loss of monomeric purity of less than about 20%, or more preferably, a loss of less than about 15%, or more preferably, a loss of less than about 10%, or more preferably, a loss of less than about 5%, or more preferably, a loss of less than about 4%, or more preferably, a loss of less than about 3%, or more preferably, a loss of less than about 2%, or more preferably, a loss of less than about 1%, or more preferably, a loss of less than about 0.6%, or more preferably, a loss of less than about 0.4%, or more preferably, a loss of less than about 0.2% of monomeric diabody (e.g., DART-A), wherein the BMW and/or LMW species of the diabody (e.g., DART-A) in the formulation is measured via SE-HPLC.

As used herein, an aqueous stabilizer solution is said to act to maintain protein stability of a diabody if its presence causes a loss of protein stability of less than about 20%, or more preferably, a loss of less than about 15%, or more preferably, a loss of less than about 10%, or more preferably, a loss of less than about 5%, or more preferably, a loss of less than about 4%, or more preferably, a loss of less than about 3%, or more preferably, a loss of less than about 2%, or more preferably, a loss of less than about 1%, or more preferably, a loss of less than about 0.6%, or more preferably, a loss of less than about 0.4%, or more preferably, a loss of less than about 0.2% of monomeric diabody (e.g., DART-A), wherein the high molecular weight and/or low molecular weight of the diabody (e.g., DART-A) in the formulation is measured via SE-HPLC.

As used herein, an aqueous stabilizer solution is said to act to inhibit or prevent non-specific adsorption of a diabody of the formulation (e.g., DART-A of the DART-A DP formulation) to the surface of a container if its presence causes a loss of diabody concentration (e.g., DART-A concentration) of less than about 20%, or more preferably, a loss of less than about 15%, or more preferably, a loss of less than about 10%, or more preferably, a loss of less than about 5%, or more preferably, a loss of less than about 4%, or more preferably, a loss of less than about 3%, or more preferably, a loss of less than about 2%, or more preferably, a loss of less than about 1%, or more preferably, a loss of less than about 0.6%, or more preferably, a loss of less than about 0.4%, or more preferably, a loss of less than about 0.2% of monomeric diabody, wherein the HMW and/or LMW species of diabody (e.g., DART-A) in the formulation is measured via SE-HPLC.

As used herein, an aqueous stabilizer solution is said to act to inhibit or prevent microbial growth within the diabody formulation (e.g., DART-A DP formulation) during its storage if its prevents or inhibits such microbial growth by more than about 10%, or more preferably, more than about 20%, or more preferably, more than about 30%, or more preferably, more than about 40%, or more preferably, more than about 50%, or more preferably, more than about 70%, or more preferably, more than about 90%, or more preferably, more than about 95%, or more preferably, more than about 97%, or more preferably, more than about 98%, or more preferably, if its presence completely prevents detectable microbial growth.

1. Stabilizer 1

Stabilizer 1 is a vehicle designed to be combined with the DART-A DP formulation to prepare a DART-A dosing solution for intravenous administration. In certain embodiments, such administration uses two infusion pumps (i.e., syringe or ambulatory pumps).

In a preferred embodiment, the DART-A DP formulation is added to a container comprising Stabilizer 1. The container is mixed and the solution is optionally diluted to prepare a dosing solution. The dosing solution is placed in a container and attached to a device for administration to a subject.

In one embodiment, being particularly suitable for pediatric administration, such aqueous stabilizer, Stabilizer 1, comprises one or more of sodium phosphate, sodium chloride, PS80, and BA. In a specific embodiment, being particularly suitable for pediatric administration, such aqueous stabilizer, Stabilizer 1, comprises sodium phosphate, sodium chloride, PS80, and does not comprise BA.

In preferred embodiments, such aqueous stabilizer will have:
(A) a sodium phosphate concentration of about 5 mM to about 30 mM, and more preferably, of about 10 mM;
(B) a sodium chloride concentration of about 100 mM to about 300 mM, and more preferably, of about 150 mM;
(C) a BA concentration of about 7.0 mg/mL to about 11.0 mg/mL, and more preferably, of about 9.0 mg/mL;
(D) a PS80 concentration of about 0.05 mg/mL to about 0.3 mg/mL, and more preferably, of about 0.10 mg/mL; and
(E) a pH of about 5.5 to about 7.0, and more preferably, a pH of about 6.0.

In a further preferred embodiment, such aqueous stabilizer will have:
(A) a sodium phosphate concentration of about 5 mM to about 30 mM, and more preferably, of about 10 mM;
(B) a sodium chloride concentration of about 100 mM to about 300 mM, and more preferably, of about 150 mM;
(C) a PS80 concentration of about 0.05 mg/mL to about 0.3 mg/mL, and more preferably, of about 0.10 mg/mL;
   and
(D) a pH of about 5.5 to about 7.0, and more preferably, a pH of about 6.0.

In a further preferred embodiment, such aqueous stabilizer will additionally comprise rHA, and more preferably, a rHA concentration of about 0.05 mg/mL to about 0.15 mg/mL, and more preferably, of about 0.10 mg/mL.

Thus, for example, such aqueous stabilizer may comprise:
(A) a sodium phosphate concentration of about 5 mM to about 30 mM, and more preferably, of about 10 mM, and:
   (1) a sodium chloride concentration of about 150 mM;
   (2) a BA concentration of about 9.0 mg/mL;
   (3) a PS80 concentration of about 0.10 mg/mL; and
   (4) a pH of about 6.0;
   and will optionally additionally comprise rHA at a concentration of about 0.10 mg/mL; or
(B) a sodium phosphate concentration of about 5 mM to about 30 mM, and more preferably, of about 10 mM, and:
   (1) a sodium chloride concentration of about 150 mM; and
   (3) a PS80 concentration of about 0.10 mg/mL; and
   (3) a pH of about 6.0; or
(C) a sodium chloride concentration of about 100 mM to about 300 mM, and more preferably, of about 150 mM, and
   (1) a sodium phosphate concentration of about 10 mM;
   (2) a BA concentration of about 9.0 mg/mL;
   (3) a PS80 concentration of about 0.10 mg/mL; and
   (4) a pH of about 6.0;
   and will optionally additionally comprise rHA at a concentration of about 0.10 mg/mL; or
(D) a sodium chloride concentration of about 100 mM to about 300 mM, and more preferably, of about 150 mM, and
   (1) a sodium phosphate concentration of about 10 mM;
   (2) a PS80 concentration of about 0.10 mg/mL; and
   (3) a pH of about 6.0; or
(E) a BA concentration of about 7.0 mg/mL to about 11.0 mg/mL, and more preferably, of about 9.0 mg/mL, and
   (1) a sodium phosphate concentration of about 10 mM;
   (2) a sodium chloride concentration of about 150 mM;
   (3) a PS80 concentration of about 0.10 mg/mL; and
   (4) a pH of about 6.0;
   and will optionally additionally comprise rHA at a concentration of about 0.10 mg/mL; or
(F) a PS80 (PS80) concentration of about 0.05 mg/mL to about 0.3 mg/mL, and more preferably, of about 0.10 mg/mL, and
   (1) a sodium phosphate concentration of about 10 mM;
   (2) a sodium chloride concentration of about 150 mM;
   (3) a BA concentration of about 9.0 mg/mL; and
   (4) a pH of about 6.0;
   and will optionally additionally comprise rHA at a concentration of about 0.10 mg/mL; or
(G) a PS80 (PS80) concentration of about 0.05 mg/mL to about 0.3 mg/mL, and more preferably, of about 0.10 mg/mL, and
   (1) a sodium phosphate concentration of about 10 mM;
   (2) a sodium chloride concentration of about 150 mM;
   (3) a pH of about 6.0; or
(H) a pH of about 5.5 to about 7.0, and more preferably, a pH of about 6.0, and
   (1) a sodium phosphate concentration of about 10 mM;
   (2) a sodium chloride concentration of about 150 mM;
   (3) a BA concentration of about 9.0 mg/mL;
   (4) a PS80 concentration of about 0.10 mg/mL; and and will optionally additionally comprise rHA at a concentration of about 0.10 mg/mL; or
(I) a pH of about 5.5 to about 7.0, and more preferably, a pH of about 6.0, and
   (1) a sodium phosphate concentration of about 10 mM;
   (2) a sodium chloride concentration of about 150 mM;
   (3) a PS80 concentration of about 0.10 mg/mL; or
(J) rHA concentration of about 0.05 mg/mL to about 0.15 mg/mL, and more preferably, of about 0.10 mg/mL, and
   (1) a sodium phosphate concentration of about 10 mM;
   (2) a sodium chloride concentration of about 150 mM;
   (3) a BA concentration of about 9.0 mg/mL; and
   (4) a PS80 concentration of about 0.10 mg/mL; and
   (5) a pH of about 6.0.

In a preferred embodiment, such aqueous stabilizer will comprise about 10 mM sodium phosphate, about 150 mM sodium chloride, about 9.0 mg/mL BA, about 0.10 mg/mL PS80, and will have a pH of about 6.0, and will additionally comprise about 0.10 mg/mL of rHA.

In another preferred embodiment, such aqueous stabilizer will comprise about 10 mM sodium phosphate, about 150 mM sodium chloride, about 0.10 mg/mL PS80, and will have a pH of about 6.0.

In a preferred embodiment, such aqueous stabilizer will be sufficient to maintain the monomeric purity of a covalently bonded diabody (e.g., CD123×CD3 diabody) preparation (such as the DART-A DP formulation) for at least about 1 day, at least about 2 days, at least about 3 days, at least about 4 days, or at least about 5 days at about 25° C.

In a preferred embodiment, such aqueous stabilizer will be sufficient to prevent or inhibit microbial growth for at least about 1 day, at least about 2 days, at least about 3 days, at least about 4 days, or at least about 5 days at about 25° C. Most preferably, such aqueous stabilizer will be sufficient to prevent or inhibit microbial growth for at least about 5 days at 25° C.

In a preferred embodiment, such aqueous stabilizer solution has a shelf-life of at least about 2 years at 2-8° C. or at least about 3 months at 25° C.

2. Stabilizer 2

Stabilizer 2 is a stabilizer designed to be used with the DART-A DP formulation for intravenous administration. In certain embodiments, such administration uses a single infusion pump configuration (i.e., syringe or ambulatory pump).

In a preferred embodiment, Stabilizer 2 is used to pre-coat a container for use with a single infusion pump. The DART-A DP formulation is added to the pre-coated container and mixed to obtain a dosing solution. The container comprising the dosing solution is attached to a device for administration to a subject.

In a second embodiment, such aqueous stabilizer, Stabilizer 2, comprises: sodium phosphate, PS80, BA, and MP. In preferred embodiments, such aqueous stabilizer will have:
(A) a sodium phosphate concentration of about 15 mM to about 25 mM, and more preferably, of about 20 mM;
(B) a BA concentration of about 11.5 mg/mL to about 15.5 mg/mL, and more preferably, of about 13.2 mg/mL;
(C) a MP concentration of about 3.5 mg/mL to about 5.5 mg/mL, and more preferably, of about 4.25 mg/mL;
(D) a PS80 concentration of about 0.1 mg/mL to about 0.4 mg/mL, and more preferably, of about 0.25 mg/mL; and
(E) a pH of about 7.7 to about 8.7, and more preferably, a pH of about 8.2.

Thus, for example, such aqueous stabilizer may comprise:
(A) a sodium phosphate concentration of about 15 mM to about 25 mM, and more preferably, of about 20 mM, and:
   (1) a BA concentration of about 13.2 mg/mL;
   (2) a MP concentration of about 4.25 mg/mL;
   (3) a PS80 concentration of about 0.25 mg/mL; and
   (4) a pH of about 8.2;
      or
(B) a BA concentration of about 11.5 mg/mL to about 15.5 mg/mL, and more preferably, of about 13.2 mg/mL, and
   (1) a sodium phosphate concentration of about 20 mM;
   (2) a MP concentration of about 4.25 mg/mL;
   (3) a PS80 concentration of about 0.25 mg/mL; and
   (4) a pH of about 8.2;
      or
(C) a MP concentration of about 3.5 mg/mL to about 5.5 mg/mL, and more preferably, of about 4.25 mg/mL, and:
   (1) a sodium phosphate concentration of about 20 mM;
   (2) a BA concentration of about 13.2 mg/mL;
   (3) a PS80 concentration of about 0.25 mg/mL; and
   (4) a pH of about 8.2;
      or
(D) a PS80 concentration of about 0.1 mg/mL to about 0.4 mg/mL, and more preferably, of about 0.25 mg/mL, and:
   (1) a sodium phosphate concentration of about 20 mM;
   (2) a BA concentration of about 13.2 mg/mL;
   (3) a MP concentration of about 4.25 mg/mL; and
   (4) a pH of about 8.2;
      or
(E) a pH of about 7.7 to about 8.7, and more preferably, a pH of about 8.2, and:
   (1) a sodium phosphate concentration of about 20 mM;
   (2) a BA concentration of about 13.2 mg/mL;
   (3) a MP concentration of about 4.25 mg/mL; and
   (4) a PS80 concentration of about 0.25 mg/mL.

In a preferred embodiment, such aqueous stabilizer will comprise about 20 mM sodium phosphate, about 13.2 mg/mL BA, about 4.25 mg/mL MP and about 0.25 mg/mL PS80, and will have a pH of about 8.2.

In a preferred embodiment, such aqueous stabilizer will be sufficient to maintain the monomeric purity of a covalently bonded diabody (e.g., CD123×CD3 diabody) preparation (such as the DART-A DP formulation) for at least about 1 day, 2 days, at least about 3 days, at least about 4 days, at least about 5 days, at least about 6 days, or at least about 7 days, at about 25° C.

In a preferred embodiment, such aqueous stabilizer will be sufficient to prevent or inhibit microbial growth for at least about 1 day, at least about 2 days, at least about 3 days, at least about 4 days, at least about 5 days, at least about 6 days, or at least about 7 days at 25° C. Most preferably, such aqueous stabilizer will be sufficient to prevent or inhibit microbial growth for at least about 7 days at 25° C.

In a preferred embodiment, such aqueous stabilizer solution has a shelf-life of at least about 2 years at 2-8° C. or at least about 3 months at 25° C.

3. Stabilizer 3

Stabilizer 3 is a vehicle designed to be combined with a bispecific diabody formulation (e.g., a DART-A DP formulation) to prepare a bispecific diabody dosing solution (e.g., a DART-A dosing solution) for intravenous administration. In certain embodiments, such administration uses a single infusion pump configuration (i.e., syringe or ambulatory pump).

In one embodiment, the bispecific diabody formulation is added to a container comprising Stabilizer 3. The container is mixed and the solution is optionally diluted to prepare a dosing solution. The dosing solution is placed in a container and attached to a device for administration to a subject.

In another embodiment, being particularly suitable for preservative-free and buffer-free administration, such Stabilizer 3 comprises sodium chloride and PS80, but does not comprise sodium phosphate or BA.

In another embodiment, such aqueous stabilizer comprises:
(A) a sodium chloride concentration of about 100 mM to about 300 mM;
(B) a PS80 concentration of about 0.05 mg/mL to about 0.3 mg/mL;
   and
(C) a pH of about 5.5 to about 7.0.

In another embodiment, such aqueous stabilizer will comprise about 150 mM sodium chloride, about 0.10 mg/mL PS80, and will have a pH of about 6.0.

In a specific embodiment, such aqueous stabilizer will be sufficient to maintain the monomeric purity of a covalently bonded diabody (e.g., CD123×CD3 diabody) preparation (such as the DART-A DP formulation) for at least about 1 day, at least about 2 days, at least about 3 days, at least about 4 days, or at least about 5 days at about 25° C.

In certain embodiments, such aqueous stabilizer is used with bacteriostatic saline.

In a specific embodiment, such aqueous stabilizer will be sufficient to prevent or inhibit microbial growth for at least about 1 day, at least about 2 days, at least about 3 days, at least about 4 days, or at least about 5 days at about 25° C. Most preferably, such aqueous stabilizer will be sufficient to prevent or inhibit microbial growth for at least about 5 days at 25° C.

In a specific embodiment, such aqueous stabilizer solution has a shelf-life of at least about 2 years at 2-8° C. or at least about 3 months at 25° C.

B. Diabody Formulations

Generally, the components of the diabody formulations (e.g., DART-A DP formulation) of the invention are supplied mixed together in unit dosage form, for example, as a liquid formulation, in a hermetically sealed container such as a vial, ampoule, or sachet indicating the quantity of active agent. The diabody formulation (such as the DART-A DP formulation) is preferably supplied as a liquid solution. Such liquid solution should be stored at between 2 and 8° C. in their original containers until ready to be administered. Where the diabody formulation is to be administered by infusion, it can be dispensed with a container, bag, or infusion bottle containing sterile saline. Where the diabody formulation is administered by injection, saline can be provided so that the ingredients may be mixed prior to administration as detailed herein. Such formulations comprise a prophylactically or therapeutically effective amount of a covalently bonded diabody comprising two, three, or four polypeptide chains. In a specific embodiment such formulations comprise a prophylactically or therapeutically effective amount of DART-A.

### V. Kits

The invention also provides a pharmaceutical pack or kit comprising one or more containers containing a diabody formulation (e.g., DART-A DP formulation), Stabilizer 1, Stabilizer 2, or Stabilizer 3. Additionally, one or more other prophylactic or therapeutic agents useful for the treatment of a disease can also be included in the pharmaceutical pack or kit. Optionally associated with such container(s) can be a notice in the form prescribed by a governmental agency regulating the manufacture, use or sale of pharmaceuticals or biological products, which notice reflects approval by the agency of manufacture, use or sale for human administration. Optionally associated with such container(s) is a product label describing the indication(s) and instructions for preparation and administration of a dosing solution comprising the diabody formulation (e.g., DART-A DP formulation), Stabilizer 1, Stabilizer 2, or Stabilizer 3.

The present invention provides kits that comprise a diabody formulation (e.g., DART-A DP formulation), Stabilizer 1, Stabilizer 2, or Stabilizer 3 and that can be used in the above methods. In such kits, the diabody formulation (e.g., DART-A DP formulation), Stabilizer 1, Stabilizer 2, or Stabilizer 3 are preferably packaged in hermetically sealed containers, such as ampoules, vials, sachets, etc. that preferably indicate the quantity of the components contained therein. The container may be formed of any pharmaceutically acceptable material, such as glass, resin, plastic, etc. The diabody formulation (e.g., DART-A DP formulation), Stabilizer 1, Stabilizer 2, or Stabilizer 3 of such kit are preferably supplied as liquid solutions. Such liquid solutions should be stored at between 2 and 8° C. in their original containers until ready to be administered. Such aqueous stabilizer solutions have a shelf-life of at least about 2 years at 2-8° C. or at least about 3 years at 25° C. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

A. Kits Particularly Suited for Use with Stabilizer 1

The invention particularly contemplates a kit, especially for use with Stabilizer 1, that comprises:
(A) a Container A that comprises a stable aqueous pharmaceutical formulation that comprises about 0.01 mg/mL to about 1 mg/mL of a covalently bonded diabody comprising two, three, or four polypeptide chains, such as a CD123×CD3 diabody (and especially, a CD123×CD3 diabody, such as DART-A), about 5 mM to about 30 mM sodium phosphate buffer, about 100 mM to about 300 mM sodium chloride and about 0.05 mg/mL to about 0.3 mg/mL PS80, wherein said formulation has a pH of about 5.5 to about 7.0; and
(B) (i) a Container B that comprises an aqueous stabilizer solution for stabilizing said diabody, said solution comprising about 5 mM to about 30 mM sodium phosphate buffer, about 100 mM to about 300 mM sodium chloride, about 7.0 mg/mL to about 11.0 mg/mL BA, about 0.05 mg/mL to about 0.3 mg/mL PS80, and said solution has a pH of about 5.5 to about 7.0; or
   (ii) a Container B that comprises an aqueous stabilizer solution for stabilizing said diabody, said solution comprising about 5 mM to about 30 mM sodium phosphate buffer, about 100 mM to about 300 mM sodium chloride, about 0.05 mg/mL to about 0.3 mg/mL PS80, and said solution has a pH of about 5.5 to about 7.0; and optionally,
(C) instructions for combining the contents of Container A and Container B for administration of the combined dosing solution to a subject in need thereof.

In a preferred embodiment, such Container A will comprise about 0.1 mg/mL of said diabody, about 10 mM sodium phosphate, about 150 mM sodium chloride, and about 0.1 mg/mL PS80, and the formulation will have a pH of about 6.0.

In a preferred embodiment, such Container B will comprise about 10 mM sodium phosphate, about 150 mM sodium chloride, about 9.0 mg/mL BA, about 0.1 mg/mL PS80, and wherein said solution has a pH of about 6.0.

In another preferred embodiment, such Container B will comprise about 10 mM sodium phosphate, about 150 mM sodium chloride, about 0.1 mg/mL PS80, and wherein said solution has a pH of about 6.0.

In a further preferred embodiment, such Container B will further comprise rHA at a concentration of about 0.05 mg/mL to about 0.15 mg/mL, and more preferably, will further comprise rHA at a concentration of about 0.1 mg/mL.

B. Kits Particularly Suited for Use with Stabilizer 2

The invention particularly contemplates a kit, especially for use with Stabilizer 2, that comprises:
(A) a Container A that comprises a stable aqueous pharmaceutical formulation that comprises about 0.01 mg/mL to about 1 mg/mL of a covalently bonded diabody comprising two, three, or four polypeptide chains, such as a CD123×CD3 diabody (and especially, a CD123×CD3 diabody such as DART-A), about 5 mM to about 30 mM sodium phosphate buffer, 100 to 300 mM sodium chloride and about 0.05 mg/mL to about 0.3 mg/mL PS80; wherein said formulation has a pH of about 5.5 to about 7.0; and
(B) a Container B that comprises an aqueous stabilizer solution for stabilizing said diabody (e.g., CD123×CD3 diabody), said solution comprising about 15 mM to about 25 mM sodium phosphate buffer, about 11.5 mg/mL to about 15.5 mg/mL BA, about 3.5 mg/mL to about 5.5 mg/mL MP and about 0.1 mg/mL to about 0.4 mg/mL PS80; wherein said solution has a pH of about 7.7 to about 8.7; and optionally
(C) instructions for combining the contents of Container A and Container B for administration of the combined dosing solution to a subject in need thereof.

In a preferred embodiment, such Container A will comprise about 0.1 mg/mL of said diabody, about 10 mM sodium phosphate, about 150 mM sodium chloride, and about 0.1 mg/mL PS80, and the formulation will have a pH of about 6.0.

In a preferred embodiment, such Container B will comprise about 20 mM sodium phosphate, about 13.2 mg/mL BA, about 4.25 mg/mL MP and about 0.25 mg/mL PS80.

C. Kits Particularly Suitable for Use with Stabilizer 3

The invention particularly contemplates a kit, especially for use with Stabilizer 3, that comprises:
(A) a Container A that comprises a stable aqueous pharmaceutical formulation that comprises about 0.01 mg/mL to about 1 mg/mL of a covalently bonded diabody comprising two, three, or four polypeptide chains, such as a CD123×CD3 diabody (and especially, a CD123×CD3 diabody, such as DART-A), about 5 mM to about 30 mM sodium phosphate buffer, about 100 mM to about 300 mM sodium chloride and about 0.05 mg/mL to about 0.3 mg/mL PS80, wherein said formulation has a pH of about 5.5 to about 7.0; and
(B) a Container B that comprises an aqueous stabilizer solution for stabilizing said diabody, said solution comprising about 100 mM to about 300 mM sodium chloride, about 0.05 mg/mL to about 0.3 mg/mL PS80, and said solution has a pH of about 5.5 to about 7.0; and optionally and optionally,
(C) instructions for combining the contents of Container A and Container B for administration of the combined dosing solution to a subject in need thereof.

In a preferred embodiment, such Container A will comprise about 0.1 mg/mL of said diabody, about 10 mM sodium phosphate, about 150 mM sodium chloride, and about 0.1 mg/mL PS80, and the formulation will have a pH of about 6.0.

In another preferred embodiment, such Container B will comprise about 150 mM sodium chloride, about 0.1 mg/mL PS80, and wherein said solution has a pH of about 6.0.

### VI. Methods of Administration

The DART-A DP formulations of the present invention may be provided for the treatment, prophylaxis, and amelioration of one or more symptoms associated with a disease, disorder or infection by administering to a subject an effective amount of a covalently bonded diabody including but not limited to a CD123×CD3 bispecific diabody of the invention, DART-A. In a preferred embodiment, such pharmaceutical formulations are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Methods of administering a diabody formulation (e.g., DART-A DP formulation) of the invention include, but are not limited to, parenteral administration (e.g., intradermal, intramuscular, intraperitoneal, intravenous and subcutaneous). In a specific embodiment, the diabody formulation (e.g., DART-A DP formulation) of the invention is administered intravenously. The diabody formulation (e.g., DART-A DP formulation) may be administered together with other biologically active agents.

Administration by infusion is preferably accomplished using an infusion pump. “Infusion pumps” are medical device that deliver fluids into a patient's body in a controlled manner, especially at a defined rate and for a prolonged period of time. Infusion pumps may be powered mechanically but are more preferably electrically powered. Some infusion pumps are “stationary” infusion pumps and are designed to be used at a patient's bedside. Others, called “ambulatory” infusion pumps, are designed to be portable or wearable. A “syringe” pump is an infusion pump in which the fluid to be delivered is held in the reservoir of a chamber (e.g., a syringe), and a moveable piston is used to control the chamber's volume and thus the delivery of the fluid. In an “elastomeric” infusion pump, fluid is held in a stretchable balloon reservoir, and pressure from the elastic walls of the balloon drives fluid delivery. In a “peristaltic” infusion pump, a set of rollers pinches down on a length of flexible tubing, pushing fluid forward. In a “multi-channel” infusion pump, fluids can be delivered from multiple reservoirs at multiple rates. A “smart pump” is an infusion pump that is equipped a computer-controlled fluid delivery system so as to be capable of alerting in response to a risk of an adverse drug interaction, or when the pump's parameters have been set beyond specified limits. Examples of infusion pumps are well-known, and are provided in, for example, [Anonymous] 2002 “General-Purpose Infusion Pumps,” Health Devices 31(10):353-387; and in U.S. Pat. Nos. 10,029,051, 10,029,047, 10,029,045, 10,022,495, 10,022,494, 10,016,559, 10,006,454, 10,004,846, 9,993,600, 9,981,082, 9,974,901, 9,968,729, 9,931,463, 9,927,943, etc.

It is preferred that the diabody formulation, particularly the DART-A DP formulation, of the invention be administered by infusion facilitated by one or more ambulatory pumps, so that the patient will be ambulatory during the therapeutic regimen.

In a preferred embodiment, the diabody formulation, particularly the DART-A DP formulation, will be administered to such subjects or patients in a treatment regimen of from about 1-7 days (e.g., a regimen of about 1 day, about 2 days, about 3 days, about 4 days, about 5 days, about 6 days, or about 7 days) or more than 7 days, or in a treatment regimen of from about 12 to 168 hours (e.g., a regimen of about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours, about 96 hours, about 102 hours, about 108 hours, about 114 hours, about 120 hours, about 126 hours, about 132 hours, about 138 hours, about 144 hours, about 150 hours, about 156 hours, about 162 hours, or about 168 hours), or more than 168 hours.

The amount of the diabody formulation of the invention which will be effective in the treatment, prevention or amelioration of one or more symptoms associated with a disorder can be determined by standard clinical techniques depending on the dosage of diabody (e.g., DART-A) to be administered. The precise amount of the diabody formulation, particularly the DART-A DP formulation, to be employed in a dosing solution will also depend on the route of administration, and the seriousness of the condition, and should be decided according to the judgment of the practitioner and each patient's circumstances. Effective dosages may be extrapolated from dose-response curves derived from in vitro or animal model test systems. Such dosages are preferably determined based upon the body weight (kg) of the recipient subject.

The stable aqueous pharmaceutical diabody formulations and stabilizers of the instant invention are particularly useful for the administration of covalently bonded diabody comprising two, three, or four polypeptide chains (e.g., DART-A) at very low concentrations (e.g., 5-500 ng/kg/day) and/or for continuous administration (e.g., by continuous infusion) over 6-96 hours, or for up to 7 days. Such embodiments are provided in further detail below.

A. Administration of Dosing Solutions Comprising a Diabody Formulation and Stabilizer 1

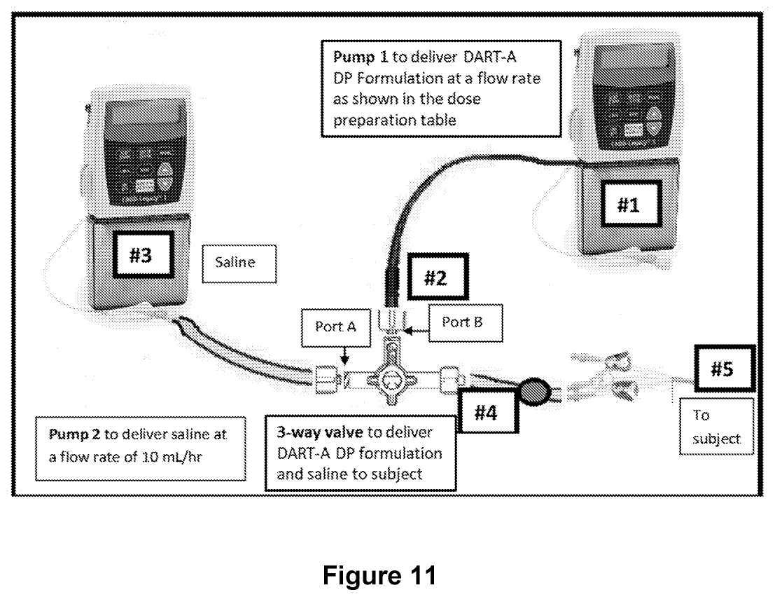

A dosing solution that comprises a diabody formulation (such as the DART-A DP formulation) and Stabilizer 1 is particularly suitable for intravenous administration using two syringe pumps or two ambulatory pumps (FIG. 11). Stabilizer 1 is particularly suitable for use in the treatment of pediatric patients, patients with low body weight, and/or patients that require a higher total IV flow rate (e.g., greater than about 5 mL/hr from the combined flow of both pumps). The diabody formulation (e.g., DART-A DP formulation) and Stabilizer 1 are combined to obtain a diabody dosing solution (e.g., a DART-A DP dosing solution). Stabilizer 1 without BA is particularly suitable for use in the treatment of pediatric patients. Such Stabilizer 1 solution is particularly suitable for administration within 24 hours after being combined with the DART-A DP formulation to obtain a DART-A dosing solution.

In such a two-pump system, a first pump (Pump 1) is used to deliver the diabody dosing solution (e.g., a DART-A DP dosing solution) to one port of a 3-way valve. Pump 1 preferably provides the dosing solution at a flow rate of 1 mL/hr or less, and in particular, at a flow rate of about 0.9 mL/hr or less, about 0.8 mL/hr or less, about 0.7 mL/hr or less, about 0.6 mL/hr or less, about 0.5 mL/hr, about 0.4 mL/hr, about 0.3 mL/hr, about 0.2 mL/hr, or about 0.1 mL/hr or less).

A second pump (Pump 2) is employed to deliver saline (0.9% sodium chloride injection USP) to a second port of the 3-way valve (for example, at a flow rate of 10 mL/hr), so as to ensure that a flow volume (e.g., 10 mL/hr) is provided that would be sufficient to prevent vein occlusion (i.e., a flow rate greater than about 5 mL/hr). The combined flows are administered intravenously to the patient. The 2-pump infusion configuration is necessary because the infusion rate of the diabody dosing solution (e.g., DART-A DP dosing solution) is preferably 1 mL/hr or less and the recommended flow rate is greater than 10 mL/hr in order to keep the central venous catheter (CVC) port open without any blood clotting. Pump 2 delivers saline at 10 mL/hr to maintain the combined flow rate of at least 10 mL/hr.

The administration of the therapeutic dosage will preferably be for at least 6 hours, at least 12 hours, at least 18 hours, at least 24 hours, or at least 30 hours (e.g., administration by continuous infusion for at least 24 hours at a flow rate of about 0.1 mL/hour to about 2.0 mL/hour).

In specific embodiments, a dosage of at least about 30 ng/kg/day to at least about 500 ng/kg/day will be administered to the patient or subject. The administration of such dosage will preferably be for at least 24 hours, at least 36 hours, at least 48 hours, at least 60 hours, at least 72 hours, at least 84 hours, at least 96 hours, or at least 7 days (i.e., 168 hours). Thus, for example, administration of the diabody dosing solution (e.g., DART-A DP dosing solution) may be by continuous infusion for at least 48 hours at a flow rate of about 0.5 mL/hour to about 6 mL/hour, for at least 96 hours at a flow rate of about 0.6 mL/hour to about 3.0 mL/hour, for at least 96 hours at a flow rate of about 0.3 mL/hour to about 3.0 mL/hour, for at least 96 hours at a flow rate of about 0.3 mL/hour, at least 7 days at a flow rate of about 0.3 mL/hour to about 3.0 mL/hour or at least 7 days at a flow rate of about 0.5 mL/hour, etc.

In order to produce a high dose concentration, an aliquot of Stabilizer 1 is mixed with an aliquot of the diabody formulation (e.g., DART-A DP formulation, so as to yield an initial diluted diabody formulation (e.g., a diluted DART-A DP formulation) with a diabody concentration of about 0.1 mg/mL. This initial diluted diabody formulation is then further diluted 1:20 with additional Stabilizer 1 (e.g., 5 mL of the initial diluted diabody formulation mixed with 95 mL of additional Stabilizer 1) and gently mixed, to yield a high dose diabody dosing solution (e.g., DART-A DP dosing solution) with a diabody concentration of about 5000 ng/mL.

In order to produce a low dose concentration, an aliquot of Stabilizer 1 is mixed with an aliquot of the diabody formulation (e.g., DART-A DP formulation), so as to yield an initial diluted diabody formulation with a diabody concentration of about 0.1 mg/mL. This initial diluted diabody formulation concentration is then further diluted 1:100 with additional Stabilizer 1 (e.g., 1 mL of the initial diluted diabody formulation mixed with 99 mL of additional Stabilizer 1) and gently mixed, to yield a secondary diluted diabody formulation with a diabody concentration of about 1000 ng/mL. This secondary diluted diabody formulation is then further diluted 1:10 with additional Stabilizer 1 (e.g., 10 mL of the secondary diluted diabody formulation mixed with 90 mL of additional Stabilizer 1) and gently mixed, to yield a low dose diabody dosing solution (e.g., a low dose DART-A DP dosing solution) with a diabody concentration at or above about 100 ng/mL.

B. Administration of DART-A DP Dosing Solutions Comprising a DART-A DP Formulation and Stabilizer 2

A dosing solution that comprises a diabody formulation (e.g., DART-A DP formulation) and Stabilizer 2 is particularly suitable for intravenous administration using a single syringe pump or a single ambulatory pump. FIG. 14 shows an example of an ambulatory pump that may be used for administration of said dosing solution. To form a dosing solution using Stabilizer 2, 40 mL of Stabilizer 2 may be added to a container, such as a 250 mL saline bag (nominal volume 270 mL), thereby forming a total volume of 310 mL. A desired amount of the diabody formulation (e.g., DART-A DP formulation) is then added to the saline bag to provide the final dosing solution, which may then be administered to the patient using a single ambulatory pump (FIG. 15).

Preferably, a dosage of at least about 30 ng/kg/day to at least about 500 ng/kg/day will be administered to the patient or subject. The administration of such dosage will preferably be for at least 24 hours, at least 36 hours, at least 48 hours, at least 60 hours, at least 72 hours, at least 84 hours, at least 96 hours, or at least 7 days (i.e., 168 hours). Thus, for example, administration of the diabody dosing solution (e.g., DART-A DP dosing solution) may be by continuous infusion for at least 48 hours at a flow rate of about 0.5 mL/hour to about 6 mL/hour, for at least 96 hours at a flow rate of about 0.6 mL/hour to about 3.0 mL/hour, for at least 96 hours at a flow rate of about 0.3 mL/hour to about 3.0 mL/hour, for at least 96 hours at a flow rate of about 0.3 mL/hour, at least 7 days at a flow rate of about 0.3 mL/hour to about 3.0 mL/hour or at least 7 days at a flow rate of about 0.5 mL/hour, etc.

Historically, if a continuous infusion over 96 hours is particularly desired, with a minimum flow rate of 60 mL/day the minimum flow rate needed to prevent vein occlusion, is about 2.5 mL/hr. However, flow rates between 0.3 mL/hour to 3.0 mL/hour have been found to be efficacious for continuous administration without causing vein occlusion. Where a lower flow rate is desired, a continuous infusion for at least 96 hours or at least 7 days at a flow rate of about 0.3 mL/hour to about 3 mL/hour is preferred. Where a lower flow rate can be used a continuous infusion for at least 96 hour at a flow rate of about 0.5 mL/hour is particularly desired. Alternatively a continuous infusion for at least 7 days (168 hours) at a flow rate of about 0.5 mL/hour is particularly desired.

C. Administration of Bispecific Diabody Dosing Solutions Comprising a Bispecific Diabody Formulation and Stabilizer 3

A dosing solution that comprises a bispecific diabody formulation (such as the DART-A DP formulation) and Stabilizer 3 is particularly suitable for intravenous administration using a single syringe pump or a single ambulatory pump. Stabilizer 3 is particularly suitable for use in the treatment of pediatric patients or in other conditions where a preservative free and buffer-free stabilizer is preferred. FIG. 14 shows an example of an ambulatory pump that may be used for administration of said dosing solution. To form a dosing solution, Stabilizer 3 may be added to a container, such as an IV bag, containing saline or bacteriostatic saline bag (nominal volume 100 mL). Bacteriostatic saline is particularly suitable for the administration of a bispecific diabody formulation with Stabilizer 3. For example, 18 mL of Stabilizer 3 can be added to an IV bag containing 70 mL of bacteriostatic saline thereby forming a total volume of 84 mL. A desired amount of the diabody formulation (e.g., DART-A DP formulation) is then added to the saline bag to provide the final dosing solution, which may then be administered to the patient using a single ambulatory pump (FIG. 15).

In certain embodiments, a dosage of at least about 30 ng/kg/day to at least about 500 ng/kg/day will be administered to the patient or subject. The administration of such dosage will be for at least 24 hours, for at least 36 hours, for at least 48 hours, for at least 60 hours, for at least 72 hours, for at least 84 hours, for at least 96 hours, or for at least 7 days (i.e., 168 hours). Thus, for example, administration of the diabody dosing solution (e.g., DART-A DP dosing solution) may be by continuous infusion for at least 48 hours at a flow rate of about 0.5 mL/hour to about 6 mL/hour, for at least 96 hours at a flow rate of about 0.6 mL/hour to about 3.0 mL/hour, for at least 96 hours at a flow rate of about 0.3 mL/hour to about 3.0 mL/hour, for at least 96 hours at a flow rate of about 0.3 mL/hour, at least 7 days at a flow rate of about 0.3 mL/hour to about 3.0 mL/hour or at least 7 days at a flow rate of about 0.5 mL/hour, etc. Where a lower flow rate is desired, a continuous infusion for at least 96 hours or at least 7 days at a flow rate of about 0.3 mL/hour to about 3 mL/hour may be used. Where a lower flow rate can be used, a continuous infusion for at least 96 hour at a flow rate of about 0.5 mL/hour or a continuous infusion for at least 7 days (168 hours) at a flow rate of about 0.5 mL/hour may be used.

### VII. Uses of the Compositions of the Invention

The diabody formulations and stabilizers of the invention are useful for the administration of a covalently bonded diabody having two, three, or four polypeptide chains to a subject in need thereof. In particular, the DART-A DP formulation may be used to treat any disease or condition associated with or characterized by the expression of CD123. In particular, the DART-A DP formulation may be used to treat hematologic malignancies. Thus, without limitation, a DART-A DP formulation may be employed in the diagnosis or treatment of AML, CIVIL, including blastic crisis of CML and Abelson oncogene associated with CIVIL (Bcr-ABL translocation), MDS, B-ALL, CLL, including Richter's syndrome or Richter's transformation of call, HCL, BPDCN, NHL, MCL, SLL, Hodgkin's lymphoma, systemic mastocytosis, and Burkitt's lymphoma. DART-A may additionally be used in the manufacture of medicaments for the treatment of the above-described conditions.

The DART-A DP formulation is particularly suitable for use in the treatment of AML, BPDCN, MDS, and T-ALL.

### VIII. Embodiments of the Invention

The invention is directed to the following embodiments E1-E129:
E1 A stable aqueous pharmaceutical formulation comprising a diabody, a sodium phosphate buffer, sodium chloride, and polysorbate 80 (PS80).
E2 The stable aqueous pharmaceutical formulation of E1, wherein the sodium phosphate has a concentration of about 5 mM to about 30 mM.
E3 The stable aqueous pharmaceutical formulation of E2, wherein the concentration of sodium phosphate is about 10 mM.
E4 The stable aqueous pharmaceutical formulation of any one of E1-E3, wherein the PS80 has a concentration of about 0.05 mg/mL to about 0.3 mg/mL.
E5 The stable aqueous pharmaceutical formulation of E4, where the concentration of PS80 is about 0.1 mg/mL.
E6 The stable aqueous pharmaceutical formulation of any one of E1-E5, wherein the sodium chloride has a concentration of about 100 mM to about 300 mM.
E7 The stable aqueous pharmaceutical formulation of E6, wherein the concentration of sodium chloride is about 150 mM.
E8 The stable aqueous pharmaceutical formulation of any one of E1-E7, wherein the formulation has a pH of about 5.5 to about 7.0.
E9 The stable aqueous pharmaceutical formulation of E8, wherein the pH is about 6.0.

The stable aqueous pharmaceutical formulation of any one of E1-E11, wherein the formulation comprises about 10 mM sodium phosphate, about 150 mM sodium chloride, and about 0.1 mg/mL PS80, and wherein the formulation has a pH of about 6.0.
E11 The stable aqueous pharmaceutical formulation of any one of E1-E11, wherein the diabody has a concentration of about 0.01 mg/mL to about 1 mg/mL.
E12 The stable aqueous pharmaceutical formulation of any one of E1-E11, wherein the concentration of diabody is about 0.1 mg/mL.
E13 The stable aqueous pharmaceutical formulation of any one of E1-E12, wherein the diabody is a covalently bonded bispecific diabody having two, three, or four polypeptide chains.
E14 The stable aqueous pharmaceutical formulation of any one of E1-E13, wherein the diabody is a CD123×CD3 diabody.
E15 The stable aqueous pharmaceutical formulation of E14, wherein the CD123×CD3 diabody comprises:
   a) a first polypeptide chain having the amino acid sequence of SEQ ID NO:42; and
   b) a second polypeptide chain having the amino acid sequence of SEQ ID NO:44; and
   wherein the first and the second polypeptide chains are covalently bonded to one another by a disulfide bond.
E16 The stable aqueous pharmaceutical formulation of any one of E1-E15, wherein the formulation comprises about 0.1 mg/mL of the diabody, about 10 mM sodium phosphate, about 150 mM sodium chloride, and about 0.1 mg/mL PS80, and wherein the pH of the formulation is about 6.0.
E17 The stable aqueous pharmaceutical formulation of any one of E1-E16, wherein the solution maintains monomeric purity of the diabody for about 3 months at 25° C.
E18 The stable aqueous pharmaceutical formulation of any one of E1-E16, wherein the solution maintains monomeric purity of the diabody for about 48 months at 2⁻8° C.
E19 A container comprising the stable aqueous pharmaceutical formulation of any one of E1-E18.
E20 The container of E19, wherein the container is a glass vial.
E21 A sealed package comprising the stable aqueous pharmaceutical formulation of any one of E1-E18 or the container of any one of E19-E20.
E22 An aqueous stabilizer solution for stabilizing a diabody, comprising sodium phosphate, PS80, benzyl alcohol (BA), and methylparaben (MP).
E23 The aqueous stabilizer solution of E22, wherein the sodium phosphate has a concentration of about 15 mM to about 25 mM.
E24 The aqueous stabilizer solution of E23, wherein the concentration of sodium phosphate is about 20 mM.
E25 The aqueous stabilizer solution of any one of E21-24, wherein the BA has a concentration of about 11.5 mg/mL to about 15.5 mg/mL.
E26 The aqueous stabilizer solution of E25, wherein the concentration of BA is about 13.2 mg/mL.
E27 The aqueous stabilizer solution of any one of E22-26, wherein the MP has a concentration of about 3.5 mg/mL to about 5.5 mg/mL.
E28 The aqueous stabilizer solution of E27, wherein the concentration of MP is about 4.25 mg/mL.
E29 The aqueous stabilizer solution of any one of E22-28, wherein the PS80 has a concentration of about 0.1 mg/mL to about 0.4 mg/mL.
E30 The aqueous stabilizer solution of E29, wherein the concentration of PS80 is about 0.25 mg/mL.
E31 The aqueous stabilizer solution of any one of E22-30, wherein the solution has a pH of about 7.7 to about 8.7.
E32 The aqueous stabilizer solution of E31, wherein the pH is about 8.2.
E33 The aqueous stabilizer solution of any one of E22-32, wherein the stabilizer solution comprises about 20 mM sodium phosphate, about 13.2 mg/mL BA, about 4.25 mg/mL MP and about 0.25 mg/mL PS80, and wherein the pH of the solution is about 8.2.
E34 The aqueous stabilizer solution of any one of E22-E33, wherein the diabody is a covalently bonded bispecific diabody having two, three, or four polypeptide chains.
E35 The aqueous stabilizer solution of any one of E22-E34, wherein the diabody is a CD123×CD3 diabody.
E36 The aqueous stabilizer solution of E35, wherein the CD123×CD3 diabody comprises:
   (a) a first polypeptide chain having the amino acid sequence of SEQ ID NO:42; and
   (b) a second polypeptide chain having the amino acid sequence of SEQ ID NO:44; and
   wherein the first and the second polypeptide chains are covalently bonded to one another by a disulfide bond.
E37 The aqueous stabilizer solution of any one of E22-E36, wherein the solution maintains monomeric purity of the diabody for about 5-7 days at about 25° C.
E38 The aqueous stabilizer solution of any one of E22-E37, wherein the solution prevents microbial growth for about 5-7 days at about 25° C.
E39 A container comprising the aqueous stabilizer solution of any one of E22-E38.
E40 The container of E39, wherein the container is a glass vial.
E41 A sealed package comprising the aqueous stabilizer solution of any one of E23-E38 or the container of any one of E39-E40.
E42 An aqueous stabilizer solution for stabilizing a diabody, comprising sodium chloride and a PS80.
E43 The aqueous stabilizer solution of E42, wherein the sodium chloride has a concentration of about 100 mM to about 300 mM.
E44 The aqueous stabilizer solution of E43, wherein the concentration of sodium chloride is about 150 mM.
E45 The aqueous stabilizer solution of any one of E42-E44, wherein the PS80 has a concentration of about 0.05 mg/mL to about 0.3 mg/mL.
E46 The aqueous stabilizer solution of E45, wherein the concentration of PS80 is about 0.10 mg/mL.
E47 The aqueous stabilizer solution of any one of E42-E46, wherein the solution has a pH of about 5.5 to about 7.0.
E48 The aqueous stabilizer solution of E47, wherein the pH is 6.0.
E49 The aqueous stabilizer solution of any one of E42-E48, wherein the solution comprise about 150 mM sodium chloride, about 0.1 mg/mL PS80, and wherein the pH of the solution is about 6.0.
E50 The aqueous stabilizer solution of any one of E42-E49, wherein the diabody is a covalently bonded bispecific diabody having two, three, or four polypeptide chains.
E51 The aqueous stabilizer solution of any one of E42-E50, wherein the diabody is a CD123×CD3 diabody.
E52 The aqueous stabilizer solution of E51, wherein the CD123×CD3 diabody comprises:
   a) a first polypeptide chain having the amino acid sequence of SEQ ID NO:42; and
   b) a second polypeptide chain having the amino acid sequence of SEQ ID NO:44; and
   wherein the first and the second polypeptide chains are covalently bonded to one another by a disulfide bond.
E53 The aqueous stabilizer solution of any one of E42-E52, wherein the solution maintains monomeric purity of the diabody for about 3-5 days at about 25° C.
E54 The aqueous stabilizer solution of any one of E42-E53, wherein the solution prevents microbial growth for about 3-5 days at about 25° C.
E55 A container comprising the aqueous stabilizer solution of any one of E42-E54.
E56 The container of E55, wherein the container is a glass vial.
E57 A sealed package comprising the aqueous stabilizer solution of any one of E42-E54 or the container of any one of E55-E56.
E58 An aqueous stabilizer solution for stabilizing a diabody, comprising one or more sodium phosphate, sodium chloride, a PS80, and a BA.
E59 The aqueous stabilizer solution of E58, wherein the sodium phosphate has a concentration of about 5 mM to about 30 mM.
E60 The aqueous stabilizer solution of E59, wherein the concentration of the sodium phosphate is about 10 mM.
E61 The aqueous stabilizer solution of any one of E58 or E59, wherein the sodium chloride has a concentration of about 100 mM to about 300 mM.
E62 The aqueous stabilizer solution of E61, wherein the concentration of the sodium chloride is about 150 mM.
E63 The aqueous stabilizer solution of any one of E58-E62, wherein the BA has a concentration of about 7.0 mg/mL to about 11.0 mg/mL.
E64 The aqueous stabilizer solution of E63, wherein the concentration of the BA is about 9.0 mg/mL.
E65 The aqueous stabilizer solution of any one of E58-E64, wherein the PS80 has a concentration of about 0.05 mg/mL to about 0.3 mg/mL.
E66 The aqueous stabilizer solution of E65, wherein the concentration of the PS80 is about 0.10 mg/mL.
E67 The aqueous stabilizer solution of any one of E58-E66, further comprising recombinant human albumin (rHA).
E68 The aqueous stabilizer solution of E67, wherein the rHA has a concentration of about 0.05 mg/mL to about 0.15 mg/mL.
E69 The aqueous stabilizer solution of E68, wherein the concentration of the rHA is about 0.10 mg/mL.
E70 The aqueous stabilizer solution of any one of E58-E69, wherein the solution has a pH of about 5.5 to about 7.0.
E71 The aqueous stabilizer solution of E70, wherein the pH is 6.0.
E72 The aqueous stabilizer solution of any one of E58-E71, wherein the solution comprises: a) about 10 mM sodium phosphate, about 150 mM sodium chloride, about 9.0 mg/mL BA, about 0.1 mg/mL PS80, and about 0.1 mg/mL rHA, and wherein the pH of the solution is about 6.0; orb) about 10 mM sodium phosphate, about 150 mM sodium chloride, about 0.1 mg/mL PS80, and wherein the pH of the solution is about 6.0.
E73 The aqueous stabilizer solution of any one of E58-E72, wherein the diabody is a covalently bonded bispecific diabody having two, three, or four polypeptide chains.
E74 The aqueous stabilizer solution of any one of E58-E73, wherein the diabody is a CD123×CD3 diabody.
E75 The aqueous stabilizer solution of E74, wherein the CD123×CD3 diabody comprises:
   (a) a first polypeptide chain having the amino acid sequence of SEQ ID NO:42; and
   (b) a second polypeptide chain having the amino acid sequence of SEQ ID NO:44; and
   wherein the first and the second polypeptide chains are covalently bonded to one another by a disulfide bond.
E76 The aqueous stabilizer solution of any one of E58-E76, wherein the solution maintains monomeric purity of the diabody for about 3-5 days at about 25° C.
E77 The aqueous stabilizer solution of any one of E58-E76, wherein the solution prevents microbial growth for about 3-5 days at about 25° C.
E78 A container comprising the aqueous stabilizer solution of any one of E58-E77.
E79 The container of E78, wherein the container is a glass vial.
E80 A sealed package comprising the aqueous stabilizer solution of any one of E58-E77 or the container of any one of E78-E79.
E81 A kit comprising:
   (a) a container A comprising a stable aqueous pharmaceutical formulation, the formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and the formulation has a pH of about 5.5 to about 7.0; and
   (b) a container B comprising an aqueous stabilizer solution for stabilizing the diabody, the solution comprising about 15 mM to about 25 mM sodium phosphate buffer, about 11.5 mg/mL to about 15.5 BA, about 3.5 mg/mL to about 5.5 mg/mL MP and about 0.1 mg/mL to about 0.4 mg/mL PS80, and the solution has a pH of about 7.7 to about 8.7; and
   (c) instructions for combining the contents of containers A and B for administration of the combined solution to a subject in need thereof.
E82 A kit comprising:
   (a) a container A comprising a stable aqueous pharmaceutical formulation, the formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and the formulation has a pH of about 5.5 to about 7.0; and
   (b) (i) a container B comprising an aqueous stabilizer solution for stabilizing the diabody, the solution comprising about 5 mM to about 30 mM sodium phosphate, about 100 mM to about 300 mM sodium chloride, about 7.0 mg/mL to about 11.0 mg/mL BA, about 0.05 mg/mL to about 0.15 mg/mL PS80, and the solution has a pH of about 5.5 to about 7.0; or
      (ii) a container B comprising an aqueous stabilizer solution for stabilizing the diabody, the solution comprising about 5 mM to about 30 mM sodium phosphate, about 100 mM to about 300 mM sodium chloride, about 0.05 mg/mL to about 0.15 mg/mL PS80, and the solution has a pH of about 5.5 to about 7.0; or
      (iii) a container B comprising an aqueous stabilizer solution for stabilizing the diabody, the solution comprising about 100 mM to about 300 mM sodium chloride, about 0.05 mg/mL to about 0.15 mg/mL PS80, and the solution has a pH of about 5.5 to about 7.0; and optionally,
   (c) instructions for combining the contents of containers A and B for administration of the combined solution to a subject in need thereof.
E83 The kit of any one of E81 or E82, wherein the diabody is a covalently bonded bispecific diabody having two, three, or four polypeptide chains.
E84 The kit of any one of E81-E83, wherein the diabody is a CD123×CD3 diabody.
E85 The kit of E84, wherein the diabody comprises:
   a) a first polypeptide chain having the amino acid sequence of SEQ ID NO:42; and
   b) a second polypeptide chain having the amino acid sequence of SEQ ID NO:44; and
   wherein the first and the second polypeptide chains are covalently bonded to one another by a disulfide bond.
E86 The kit of any of E81-E85, wherein the stable aqueous pharmaceutical formulation in container A comprises about 0.1 mg/mL of the diabody, about 10 mM sodium phosphate, about 150 mM sodium chloride, and about 0.1 mg/mL PS80, and wherein the formulation has a pH of about 6.0.
E87 The kit of any one of E81, or E83-E86, wherein the aqueous stabilizer solution in container B comprises about 20 mM sodium phosphate, about 13.2 mg/mL BA, about 4.25 mg/mL MP and about 0.25 mg/mL PS80, and wherein the solution has a pH of about 8.2.
E88 The kit of any one of E82-E86, wherein the wherein the aqueous stabilizer solution in container B comprises: i) about 10 mM sodium phosphate, about 150 mM sodium chloride, about 9.0 mg/mL BA, about 0.1 mg/mL PS80, and wherein the solution has a pH of about 6.0; or
   ii) about 10 mM sodium phosphate, about 150 mM sodium chloride, about 0.1 mg/mL PS80, and wherein the solution has a pH of about 6.0; or
   (iii) about 150 mM sodium chloride, about 0.1 mg/mL PS80, and wherein said solution has a pH of about 6.0.
E89 The kit of any one of E82-E86, wherein the aqueous stabilizer solution further comprises rHA at a concentration of about 0.05 mg/mL to about 0.3 mg/mL.
E90 The kit of E89 wherein the concentration of rHA is about 0.1 mg/mL.
E91 The kit of any one of E81-E90, wherein the subject is a human patient.
E92 The kit of any one of E81-E91, wherein the container A and container B are glass vials.
E93 A sealed package comprising the kit and optionally instructions for storage and/or use of the kit of any one of E81-E92.
E94 A method of administering a diabody to a subject in need thereof comprising using the kit of any one of E81, E83-E85, E87, or E91-E93-, wherein the aqueous stabilizer solution of the container B comprises sodium phosphate, PS80, BA, MP, and has a pH of about 7.7 to about 8.7;
   and wherein in the method:
   (a) the aqueous stabilizer solution of container B is placed in a container C and mixed;
   (b) the stable aqueous pharmaceutical formulation of container A is placed in the container C and mixed to obtain a dosing solution;
   (c) the container C containing the dosing solution is attached to a device for administration to the subject.
E95 The method of E94, wherein the container C comprises saline for intravenous infusion.
E96 A method of administering a diabody to a subject in need thereof comprising using the kit of any one of E82-E86, or E88-E93, wherein the aqueous stabilizer solution of the container B comprises one or more of sodium phosphate, sodium chloride, PS80, and BA, and optionally rHA, and has a pH of about 5.5 to about 7.0;
   and wherein in the method:
   (a) the stable aqueous pharmaceutical formulation of the container A is placed in the aqueous stabilizer solution of the container B and mixed to obtain a dosing solution;
   (b) optionally diluting the dosing solution;
   (c) placing the dosing solution in a container C; and
   (d) attaching the container C containing the final dosing solution to a device for administration to the subject.
E97 The method of E96, wherein the container C comprises saline or bacteriostatic saline for intravenous infusion.
E98 The method of any one of E94-E97, wherein the administration is by infusion pump.
E99 The method of any one of E94-E98, wherein the administration is ambulatory.
E100 The method of any one of E94-E98, wherein the device is a single ambulatory pump.
E101 The method of any one of E94-E98, wherein the device is a dual ambulatory pump.
E102 The method of any one of E94-E98, wherein the device is a syringe pump.
E103 The method of any one of E94-E102, wherein the administration is by continuous infusion for at least about 24 hours.
E104 The method of any one of E94-E102, wherein the administration is by continuous infusion for at least about 48 hours.
E105 The method of any one of E94-E102, wherein the administration is by continuous infusion for at least about 96 hours.
E106 The method of any one of E94-E102, wherein the administration is by continuous infusion for at least about 7 days.
E107 The method of any one of E94-E106, wherein the administration occurs at a flow rate of about 0.10 mL/hour to about 2.5 mL/hour.
E108 The method of any one of E94-E106, wherein the administration occurs at a flow rate of about 0.5 mL/hour to about 10.0 mL/hour.
E109 The method of any one of E94-E102, wherein the administration is by continuous infusion for at least 24 hours at a flow rate of about 0.1 mL/hour to about 2.0 mL/hour.
E110 The method of any one of E94-E102, wherein the administration is by continuous infusion for at least 48 hours at a flow rate of about 0.5 mL/hour to about 6 mL/hour.
E111 The method of any one of E94-E102, wherein the administration is by continuous infusion for at least 96 hours at a flow rate of about 0.6 mL/hour to about 3.0 mL/hour.
E112 The method of any one of E94-E102, wherein the administration is by continuous infusion for at least 96 hours at a flow rate of about 0.3 mL/hour to about 3.0 mL/hour.
E113 The method of any one of E94-E102 or E112, wherein the administration is by continuous infusion for at least 96 hours at a flow rate of about 0.5 mL/hour.
E114 The method of any one of E94-E102, wherein the administration is by continuous infusion for at least 7 days at a flow rate of about 0.3 mL/hour to about 3.0 mL/hour.
E115 The method of any one of E94-E102 or E114, wherein the administration is by continuous infusion for at least 7 days at a flow rate of about 0.5 mL/hour.
E116 The method any one of E94-E115, wherein the flow rate prevents vein occlusion in the subject.
E117 The method of any one of E94-E116, wherein the CD123×CD3 diabody is administered to the subject at a treatment dosage selected from the group consisting of 30-500 ng/kg/day.
E118 The method of any one of E94-E95, E97-E99, E102-E117, wherein the dosing solution comprises 40 mL of aqueous stabilizer solution.
E119 The method of any one of E94-E95, E97-E99, E102-E118, wherein the dosing solution comprises about 0.03 mg/mL to about 0.04 mg/mL PS80, about 1.7 mg/mL to about 2.1 mg/mL BA, and about 0.55 mg/mL to about 0.7 mg/mL MP.
E120 The method of any one of E96-E118, wherein the dosing solution comprises about 100 mM to about 300 mM sodium chloride, about 0.05 mg/mL to about 0.15 mg/mL PS80, and the solution has a pH of about 5.5 to about 7.0.
E121 The method of any one of E94-E120, wherein the patient is a human subject.
E122 A method of treating a hematologic malignancy comprising administering a diabody to a subject in need thereof, according to the method of administering of any one of E94-E121 or using the kit of any one of E81-E92.
E123 The use of the kit of any one of E81-E92 for the treatment of a hematologic malignancy.
E124 The method of E122, or the use of E123, wherein the hematologic malignancy is selected from the group consisting of: acute myeloid leukemia (AML), chronic myelogenous leukemia (CML), including blastic crisis of CIVIL and Abelson oncogene associated with CIVIL (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.
E125 The method or the use of E124, wherein the hematologic malignancy is AML.
E126 The method or the use of E124, wherein the hematologic malignancy is BPDCN.
E127 The method or the use of E124, wherein the hematologic malignancy is MDS.
E128 The method or the use of E124, wherein the hematologic malignancy is T-ALL.
E129 The method or the use of any one of E122-E128, wherein the subject is a human subject.

### EXAMPLES

Having now generally described the invention, the same will be more readily understood through reference to the following examples, which are provided by way of illustration and are not intended to be limiting of the present invention unless specified.

### Example 1

### Development of a Stable Aqueous Pharmaceutical Formulation Containing a CD123×CD3 Diabody

As disclosed above, the CD123×CD3 diabody, DART-A, is a bispecific monovalent diabody that is capable of simultaneous binding to CD123 and CD3. DART-A consists of a first polypeptide chain having the amino acid sequence of SEQ ID NO:42 and a second polypeptide chain having the amino acid sequence of SEQ ID NO:44 and have the general structure show in FIG. 1. A stable aqueous pharmaceutical formulation comprising DART-A, the DART-A DP formulation, was developed as a liquid formulation in vials.

### 1.1. Development of the DART-A DP Formulation—Study Design and Results

The analytical testing methods used during the development of the DART-A DP formulation are listed below in Table 5.

TABLE 5
Analytical Tests for DART-A DP Formulation

| Attribute Tested | Method* |
|---|---|
| Protein Concentration | Absorbance at 280 nm |
| Solution Turbidity | Absorbance at 320 nm |
| Sub-visible Particle Count | Light Obscuration Method |
| | (HIAC) |
| Thermal Stability | Differential Scanning |
| | Calorimetry |
| Charge Variants | Capillary Isoelectric |
| | Focusing |
| pH | pH |
| Appearance | Visual Appearance |
| Stability by Peptide Mapping | LC-ESIMS |
| Protein Aggregation | SE-HPLC |

*All methods are detailed below in Example 5.

Three studies were performed to provide a DART-A DP formulation for toxicology animal studies and clinical use. These studies evaluated the following formulation attributes:
(1) Effect of surfactant levels on DART-A stability and aggregation
(2) Effect of excipient and protein concentration on DART-A aggregation and particle formation
(3) DART-A peptide mapping via mass spectrometry analytical techniques

### 1.2. Effect of Surfactant Levels on DART-A Stability and Degradation

To formulate DART-A to a 0.1 mg/mL concentration, the DART-A was diluted in formulation buffer (10 mM sodium phosphate and 150 mM sodium chloride at pH 6.1). Initial sub-visible particle formation and solution turbidity was observed to be acceptable. However, one month after shipping, increased turbidity and elevated sub-visible particle levels that exceeded the original release specifications were observed. A forced agitation study was conducted on two development lots with and without PS80 (0.1 mg/mL) to determine the cause of particle formation and change in appearance. During the forced agitation study, the DART-A samples were held at 2-8° C. and agitated at 600 rpm over 24 hours.

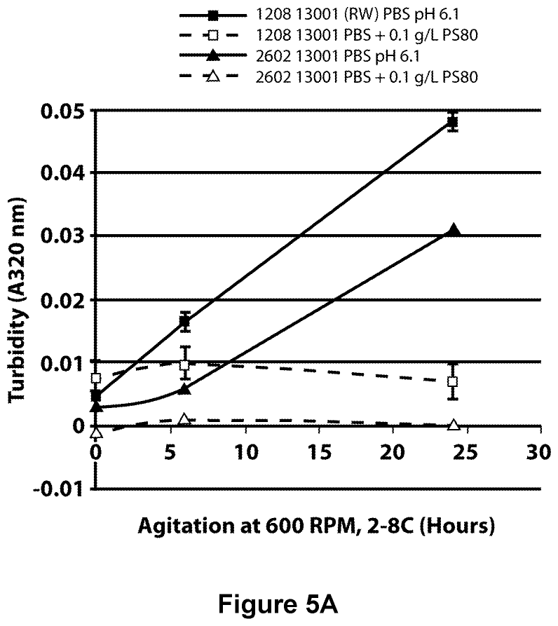

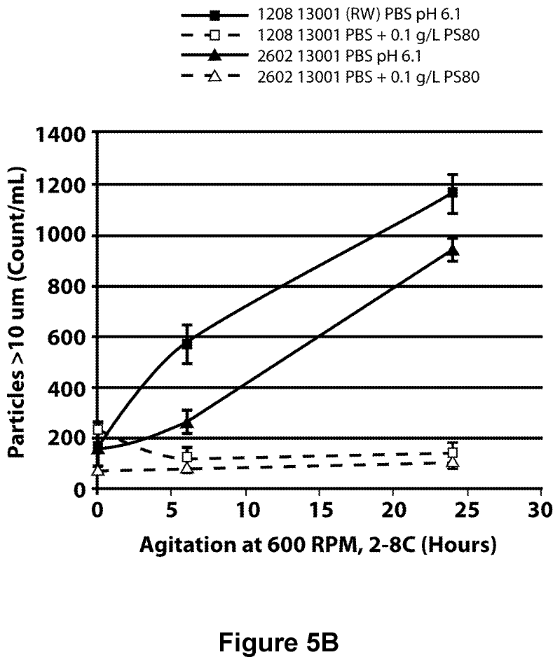

FIGS. 5A-5B show that the PS80 containing formulation shows low turbidity of the DART-A after 24-hour agitation. When PS80 is not present, the turbidity of the DART-A DP formulation increases three to five-fold. This trend is also observed in the sub-visible particle count data where PS80-free diabody formulations have a five to six-fold higher number of particles ≥10 μm compared to solutions that contained PS80.

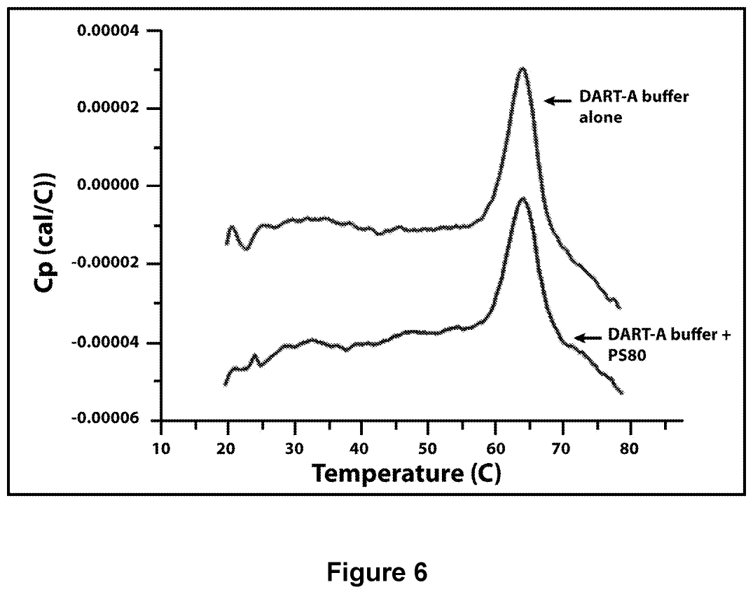

Thermal stability of a PS80 containing formulation was evaluated using differential scanning calorimetry (DSC) as shown in FIG. 6. FIG. 6 shows similar DSC profiles in the presence and absence of PS80. The presence of PS80 does not affect the thermostability of the DART-A DP formulation.

To further characterize DART-A compatibility in the presence of PS80, an accelerated stress study was performed to determine if DART-A aggregation is temperature dependent and if aggregation affects DART-A potency. In the study, DART-A with and without PS80 was incubated at 25±2° C. and 40±2° C. and compared to samples stored at 5±3° C. The accelerated stress study also assessed the impact of residual peroxide (1.0 μeq/L) on DART-A protein stability as polysorbate undergoes auto-oxidation resulting in peroxide formation, which may potentially cause oxidative degradation of the protein. The results of the study are presented in Tables 6-8 below.

TABLE 6
DART-A SE-HPLC Results After Incubation for Six Weeks

| Formulation | Charge Variant | 5 ± 3° C. | 25 ± 2° C. | 40 ± 2° C. |
|---|---|---|---|---|
| PBS | % Main Peak | 99.2 | 98.9 | 97.0 |
| pH 6.1 | % HMW | 0.8 | 1.0 | 1.8 |
| | % LMW | 0.1 | 0.2 | 1.2 |
| PBS + PS80 | % Main Peak | 99.7 | 99.7 | 98.1 |
| pH 6.1 | % HMW | 0.3 | 0.2 | 1.0 |
| | % LMW | 0.0 | 0.0 | 1.0 |
| PBS + PS80 + | % Main Peak | 99.6 | 99.5 | 98.0 |
| H₂O₂ pH | % HMW | 0.2 | 0.3 | 0.9 |
| 6.1 | % LMW | 0.2 | 0.1 | 1.0 |

Table 6 shows that the addition of PS80 reduces HMW formation after six-week storage at 5±3° C., 25±2° C., and 40±2° C. After a six-week incubation at 40° C., all three samples showed a slight decrease in monomeric DART-A protein main peak percentage. These decreases are associated with slight increases in both HMW and LMW species.

TABLE 7
DART-A Concentration and Turbidity After Incubation for Six

| Formulation | Charge Variant | 5 ± 3° C. | 25 ± 2° C. | 40 ± 2° C. |
|---|---|---|---|---|
| PBS | Concentration | 0.089 | 0.095 | 0.085 |
| PH 6.1 | Turbidity (A320) | 0.008 | 0.002 | 0.002 |
| PBS + PS80 | Concentration | 0.097 | 0.097 | 0.087 |
| PH 6.1 | Turbidity (A320) | 0.007 | 0.001 | 0.006 |
| PBS + PS80 + | Concentration | 0.097 | 0.096 | 0.087 |
| H₂O₂ pH 6.1 | Turbidity (A320) | 0.003 | 0.002 | 0.016 |

Table 7 shows no changes in solution turbidity following incubation at 25±2° C. or 40±2° C. for either the PBS or PBS+PS80 formulations. However, the formulation containing hydrogen peroxide showed increased turbidity at 40±2° C. For all three formulations, incubation at 40±2° C. for six weeks produced a similar decrease in protein concentration.

TABLE 8
DART-A Charge Variants After Incubation for Six Weeks

| Incubation | | Reference | PBS | PBS + PS80 | PBS + PS80 + |
|---|---|---|---|---|---|
| Temperature | Charge Variant | Standard | pH 6.1 | pH 6.1 | H₂O₂ pH 6.1 |
| 5 ± 3° C. | % Acidic | 19.8 | 20.6 | 21.6 | 20.6 |
| | % Main Peak | 75.3 | 75.9 | 76.2 | 77.5 |
| | % Basic | 4.9 | 3.5 | 2.3 | 1.9 |
| 25 ± 2° C. | % Acidic | N/A | 21.9 | 23.1 | 22.8 |
| | % Main Peak | N/A | 75.0 | 74.7 | 75.5 |
| | % Basic | N/A | 3.1 | 2.2 | 1.7 |
| 40 ± 2° C. | % Acidic | N/A | 46.5 | 46.2 | 45.8 |
| | % Main Peak | N/A | 53.5 | 53.8 | 54.2 |
| | % Basic | N/A | 0.0 | 0.0 | 0.0 |

Table 8 shows no significant differences in charge variants across all three formulations when incubated at both 5±3° C. and 25±2° C. Incubation at 40±2° C. produced a significant increase in acidic variants; however, this increase was consistent across all three formulations.

The results of the accelerated stress stability study suggest that PS80 does impact protein stability when exposed to typical storage or accelerated stress temperatures. This was confirmed in the results of the forced agitation study, which showed that the presence of PS80 limits increases of turbidity and sub-visible particles of DART-A in formulation which could occur during typical shipping and handling. Based on these results, 0.1 mg/mL PS80 was added to the DART-A DP formulation (10 mM sodium phosphate and 150 mM sodium chloride at pH 6.0).

### 1.3. Effect of Long-Term Storage on DART-A Structure and Function

In a second study, the DART-A formulation (10 mM sodium phosphate, 150 mM NaCl and 0.1 mg/mL PS80 buffer) at a concentration of 0.1 mg/mL was evaluated during long term storage at 5±3° C. and 25±2° C. to determine how the formulation maintains protein structure and function. The DART-A formulation composition is listed below in Table 9.

TABLE 9
DART-A Formulation Composition

| | | DART- | Sodium | Sodium | | |
|---|---|---|---|---|---|---|
| | | A | Phosphate | Phosphate | Sodium | |
| Name | Code | Conc. | Monobasic | Dibasic | Chloride | PS80 |
| Formulation | DART- | 0.1 | 1.1 | 0.26 | 8.8 | 0.1 |
| 1 | A | | | | | |

The DART-A formulation was filled in vials according to the proposed final clinical presentation (5 mL solution volume in a 5 mL Type I borosilicate glass vial) and stored inverted according to the stability matrix below in Table 10.

TABLE 10
Stability Matrix for All Formulations
Including Temperature Conditions and Time Points

| Temperature | Time Points (Months) |
|---|---|
| 5 ± 3° C. | 0, 1, 3, 6, 9, 12, 18, 24, 36, 48 |
| 25 ± 2° C. | 0, 1, 3, 5, 6 |

The stability results of the 0.1 mg/mL DART-A in the 150 mM NaCl+PS80 formulation is shown below in Table 11 and Table 12.

TABLE 11
DART-A Stability Results After Incubation at 5 ± 3° C.

| | | Results | Time (Months) | | | | |
|---|---|---|---|---|---|---|---|
| Formulation | Test | Reported | 0 | 12 | 24 | 36 | 48 |
| 0.1 mg/mL | Potency | Relative | 104 | 105 | 99 | 96 | 101 |
| DART-A in | (CD123-Binding) | Potency | | | | | |
| 150 mM | Potency | Relative | 109 | 106 | 100 | 102 | 97 |
| NaCl, 0.01% | (CD3-Binding) | Potency | | | | | |
| PS80 | Reduced CE-SDS | Product | 97.3 | 96.5 | 97.2 | 96.4 | 96.4 |
| | | Purity | | | | | |
| | SE-HPLC ^{A} | % Main | 98.6 | 98.4 | 98.8 | 98.8 | 98.7 |
| | | Peak | | | | | |
| | | % HMW | 1.4 | 1.4 | 1.2 | 1.1 | 1.2 |
| | | % LMW | 0.0 | 0.2 | 0.1 | 0.1 | 0.0 |
| | Particle Count | ≥2 μm | 11,048 | 18,858 | 4,592 | 2,226 | 2,368 |
| | (per vial) | ≥10 μm | 30 | 227 | 29 | 16 | 20 |
| | | ≥25 μm | 0 | 8 | 1 | 0 | 0 |

^{A} SE-HPLC: % HMW = % High Molecular Weight species,
% LMW = % Low Molecular Weight species

The above results show that storage at the recommended temperature of 5±3° C. does not impact DART-A structure and function after 48 months. The formulation limits DART-A aggregation and degradation as observed in the subvisible particle, SE-HPLC and reduced/non-reduced CE-SDS results. DART-A potency does not significantly change as confirmed by the CD3 and CD123 binding results.

TABLE 12
DART-A Stability Results After Incubation at 25 ± 2° C.

| | | Results | Time (Months) | | | | |
|---|---|---|---|---|---|---|---|
| Formulation | Test | Reported | 0 | 1 | 3 | 5 | 6 |
| 0.1 mg/mL | Potency | Relative | 104 | 96 | 99 | 93 | 82 |
| DART-A in | (CD123-Binding) | Potency | | | | | |
| 150 mM | Potency | Relative | 109 | 99 | 94 | 78 | 78 |
| NaCl, 0.01% | (CD3-Binding) | Potency | | | | | |
| PS80 | Reduced CE-SDS | Product | 97.3 | 96.6 | 95.9 | 95.8 | 94.9 |
| | | Purity | | | | | |
| | Non-Reduced | Product | 97.1 | 95.7 | 94.7 | 91.8 | 92.4 |
| | CE-SDS | Purity | | | | | |
| | SE-HPLC ^{A} | % Main | 98.6 | 98.6 | 98.6 | 98.4 | 98.5 |
| | | Peak | | | | | |
| | | % HMW | 1.4 | 1.3 | 1.3 | 1.5 | 1.2 |
| | | % LMW | 0.0 | 0.1 | 0.1 | 0.1 | 0.2 |
| | Particle Count | ≥2 μm | 11,048 | NS | NS | NS | 4,574 |
| | (per vial) | ≥10 μm | 30 | NS | NS | NS | 63 |
| | | ≥25 μm | 0 | NS | NS | NS | 1 |

^{A} SE-HPLC: % HMW = % High Molecular Weight species,
% LMW = % Low Molecular Weight species

The above results show that after incubation at an accelerated temperature of 25±2° C. for six months, the DART-A formulation still meets United States Pharmacopeia (USP) specifications for sub-visible particle counts. In addition, the SE-HPLC aggregation and CE-SDS degradation profiles compare favorably to DART-A stored at 5±3° C.

Based on the combined stability results, it was concluded that the 10 mM sodium phosphate, 150 mM NaCl and 0.1 mg/mL PS80 buffer provides acceptable protein stability for 48 months under recommended storage (5±3° C.) temperatures and for three months under accelerated temperature (25±2° C.) conditions. Accordingly, the 10 mM sodium phosphate at pH 6.0, 150 mM sodium chloride, 0.1 mg/mL PS80 buffer was selected for the clinical and commercial DART-A DP formulation.

The DART-A DP formulation is a clear to slightly opalescent, colorless to pale yellow or pale brown sterile, stable aqueous pharmaceutical solution with a DART-A concentration of about 0.01 mg/mL to about 1 mg/mL in a buffer composed of about 5 mM to about 15 mM sodium phosphate at pH 6.0, about 100 mM to about 200 mM sodium chloride, and about 0.05 mg/mL to about 0.3 mg/mL PS80.

### 1.4. DART-A Stability Analysis by Peptide Mapping

The stability of the formulated DART-A (i.e., DART-A DP formulation) was further evaluated with liquid chromatography coupled with electrospray ionization mass spectrometry (LC-ESIMS) by comparing a reference standard with the DART-A DP formulation stored at 2-8° C. for 24 months. The samples were analyzed as an intact molecule and its tryptic peptides. The individual test results are summarized below.

### 1.5. Intact DART-A

The mass in Daltons (Da) of the intact DART-A was obtained by deconvolving the multiply charged ions in its ESI mass spectrum. The dominant components in the reference standard and aged formulation spectra showed measured masses at 58,897 Da and 58,898 Da, respectively. These results matched closely with the theoretical mass of 58,898 Da, which was derived from the amino acid sequence of DART-A along with the conversion of N-terminal glutamine to pyroglutamate. A second observed component in the spectra from each sample had a mass of 128 Da less than the dominant component; this coincides with the clipping of the C-terminal lysine. The minor components in the ESI mass spectra are likely due to adduct ion formation and other modifications. These modifications to DART-A were further studied through tryptic peptide mapping.

### 1.6. Tryptic Peptide Mapping

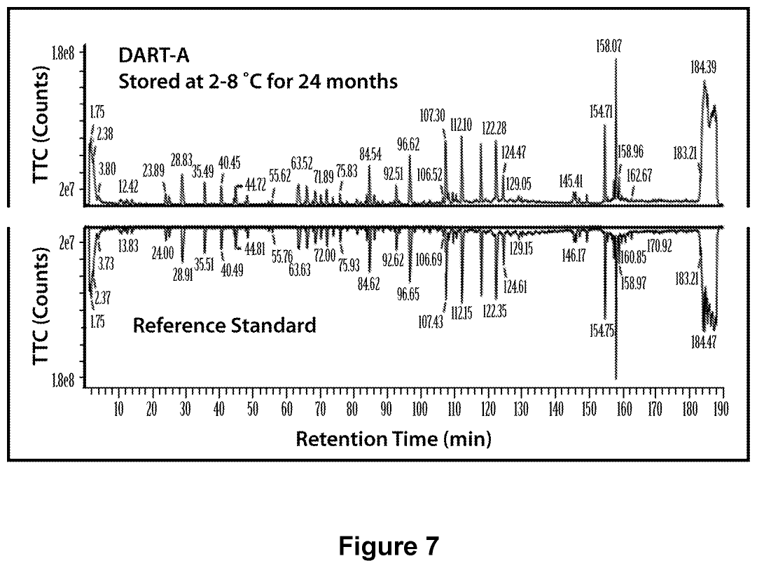

In this study, DART-A was digested into tryptic peptides to examine potential modifications to the DART-A. The peptides and their modifications at the amino acid level were identified with the accurate mass measurement of precursor ions and fragment ions. The results are shown below in FIG. 7. The results in FIG. 7 show that besides N-terminal pyroE formation and C-terminal lysine clipping, the levels of other modifications, such as deamidation and oxidation, revealed by the peptide mapping are minor for both samples. The aged DART-A is consistent with its reference standard in terms of types and their corresponding degrees of modifications.

### 1.7. DART-A DP Formulation Long Term Stability

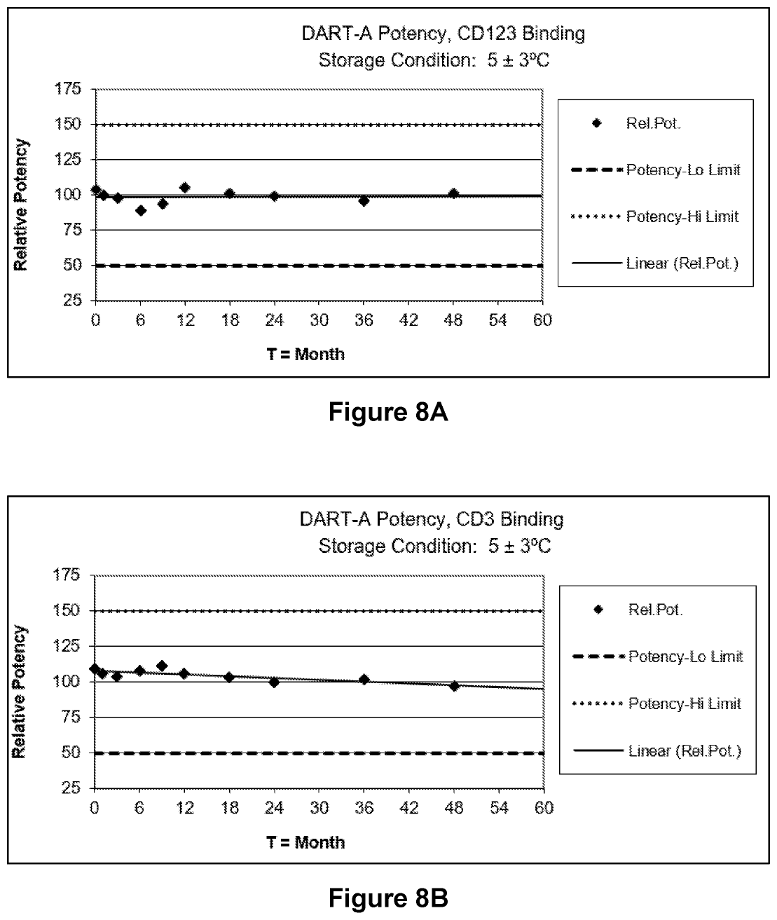

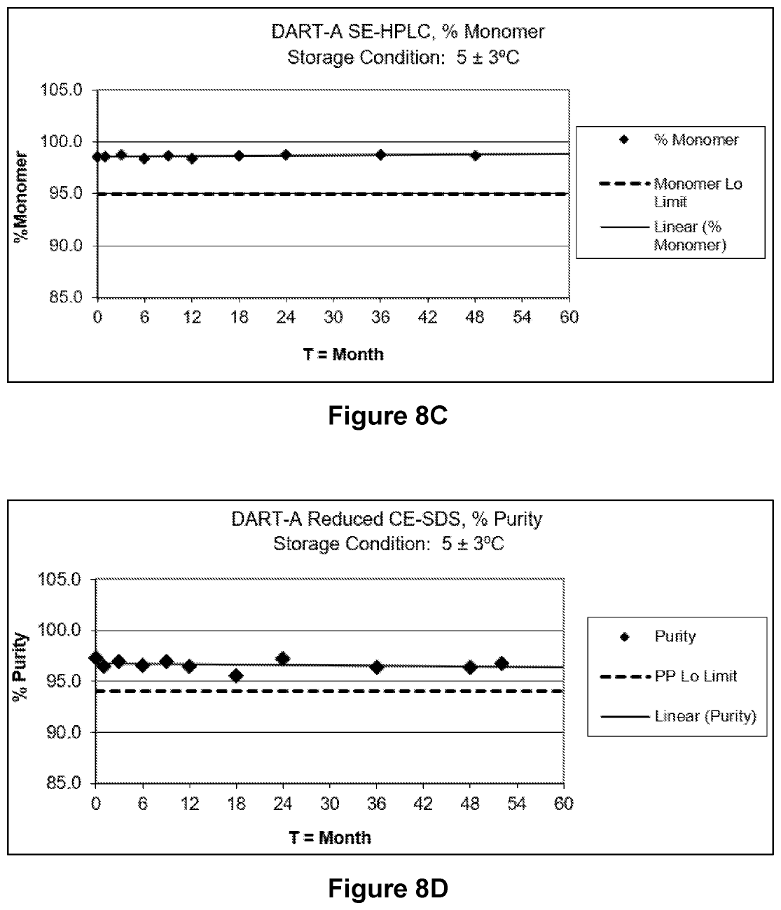

The stability of the DART-A DP formulation stored at 2-8° C. for 48 months was evaluated by ELISA binding, SE-HPLC and CE-SDS to identify any reduction in monomers and/or formation of HMW aggregates and/or LMW degradation products that may affect DART-A functionality. The results of these studies are plotted in FIGS. 8A-8D and show that the DART-A DP formulation exhibits acceptable physical stability on long term storage at the recommended storage conditions. Based on the studies performed to date, mass spectrometry-based characterization and the GMP lot stability, the DART-A DP formulation was recommended for clinical studies and commercialization.

### Example 2

### Development of an Aqueous Stabilizer Solution for Administration of the DART-A DP Formulation

As described above, a preferred aqueous stabilizer solution for stabilizing DART-A was developed as a liquid formulation in vials. However, DART-A is active at very low concentrations and the DART-A DP formulation is therefore to be diluted prior to administration. For this purpose, the DART-A DP formulation is diluted in a custom stabilizer solution to inhibit non-specific binding of DART-A to syringes or ambulatory cassettes and IV tubing during dose preparation and administration as the DART-A DP formulation is administered as a continuous 24-hour intravenous (IV) infusion.

A first stable aqueous stabilizer solution for stabilizing DART-A, Stabilizer 1, was prepared and is composed of 10 mM sodium phosphate, 150 mM sodium chloride, 0.1 mg/mL PS80, 9.0 mg/mL BA and 0.1 mg/mL rHA at pH 6.0. Stabilizer 1 is designed to be combined with the DART-A DP formulation for intravenous administration using two syringe pumps or two ambulatory pumps.

Modifications to the DART-A DP formulation and Stabilizer 1 were evaluated to enable single pump administration. As described in detail below, the new aqueous stabilizer solution, Stabilizer 2, was developed based on alternate preservative screening studies to determine the effective concentration of preservatives that would: (a) be considered safe at the proposed KVO (keep vein open) flow rate, (b) inhibit microbial growth in the dose prepared solution and (c) not affect DART-A DP formulation stability.

### 2.1 Development of Stabilizer 1 and Stabilizer 2 Solutions—Study Designs and Results

The list of analytical testing methods used during the development of the Stabilizer 1 and Stabilizer 2 are listed below in Table 13.

TABLE 13
Analytical Tests for Stabilizer 1 and Stabilizer 2

| Attribute Tested | Method* |
|---|---|
| Protein Concentration | Absorbance at 280 nm |
| Sub-visible Particle Count | Light Obscuration Method |
| | (HIAC) |
| pH | pH |
| Appearance | Visual Appearance |
| Protein Recovery ^{A} | ELISA (rhIL3Rα) |
| Protein Aggregation | FLR SE-HPLC |
| Benzyl Alcohol Recovery | RP-HPLC |
| Potency | CD3 and CD123 Binding |
| Osmolality | Osmolality |

^{A} The ELISA quantitation assay is an assay that can quantify ng/mL concentrations of DART-A.
*All methodsare detailed below in Example 5.

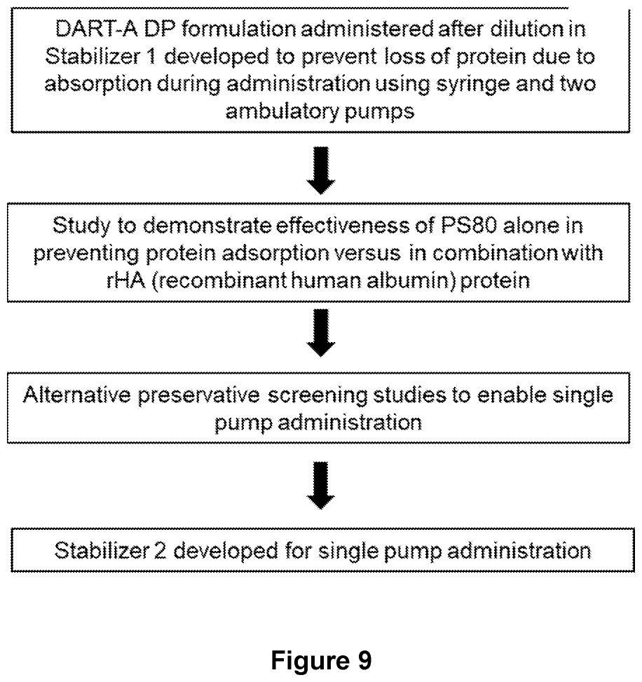

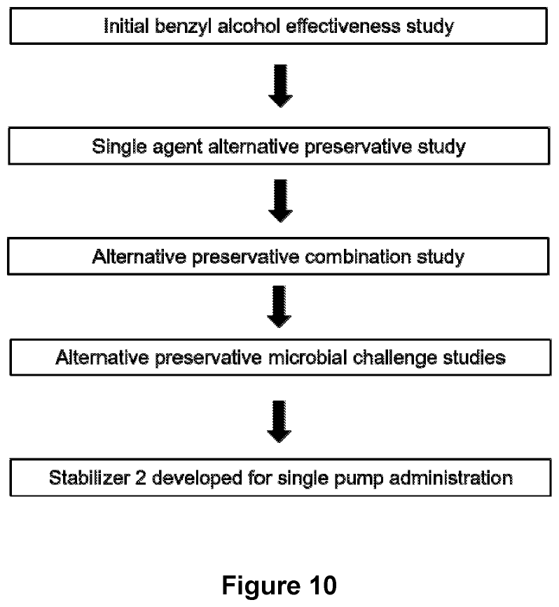

The formulation development for the stabilizer was performed in stages as described in FIGS. 9-10.

Stabilizer 1 is composed of 10 mM sodium phosphate, 150 mM sodium chloride, 0.1 mg/mL PS80, 9.0 mg/mL BA, and 0.1 mg/mL rHA at pH 6.0 and is designed to be combined with the DART-A DP formulation for intravenous administration using two syringe pumps or two ambulatory pumps. BA serves to inhibit microbial growth during storage for continuous administration. However, BA may cause increased protein aggregation and particle formation. To inhibit potential particle formation and adsorption of protein to the administration flow path surfaces, PS80 and rHA were added to the stabilizer solution. To transition from a two-pump administration configuration to a single ambulatory pump configuration, additional formulation development studies evaluating potential blocking agents and alternative preservatives were needed. These studies are described in further detail below.

### 2.2. Selection of Blocking Agent

The Phase 1 Stabilizer 1 solution contains 0.1 mg/mL rHA to limit non-specific DART-A protein binding to the surfaces of the administration components. rHA is a common blocking agent that acts by binding to the reactive sites before protein addition. To lower the manufacturing cost of goods (COGs), the surfactant PS80 (which was already in solution) was evaluated as a substitute blocking agent. FIG. 9 summarizes the studies performed to evaluate the effectiveness of PS80 alone in inhibiting particle formation and adsorption.

Four levels of PS80 were tested during this study to evaluate the blocking activity of the surfactant. Briefly, test stabilizer solutions (10 mM sodium phosphate at pH 6.0, 150 mM sodium chloride, 1.6 mg/mL BA and 0, 0.05, 0.1, or 0.2 mg/mL PS80) were added to a normal saline containing IV bag. After thorough mixing, the DART-A DP formulation was added to the same IV bag. The bag containing the dosing solution was incubated at 25±2° C. and samples were collected at 0, 24 and 48 hours (the longest proposed administration time). Each sample was analyzed using FLR/SE-HPLC and verified using ELISA to quantify DART-A protein recovery. The results of the study can be seen below in Table 14.

TABLE 14
DART-A Protein Recovery within Various PS80 Containing
Formulations

| | T₀ | | 24 Hr | | 48 Hr | |
|---|---|---|---|---|---|---|
| PS80 Conc. | % Recovery | | % Recovery | | % Recovery | |
| (mg/mL) | FLR | ELISA | FLR | ELISA | FLR | ELISA |
| 0 + 1.6 | ND | 37.9 | ND | <LLOQ | ND | <LLOQ |
| mg/mL BA | | | | | | |
| 0.05 + 1.6 | 111.8 | 88.0 | 117.3 | 88.0 | 126.0 | 89.9 |
| mg/mL BA | | | | | | |
| 0.1 + 1.6 | 97.5 | 96.1 | 87.7 | 91.6 | 99.1 | 88.8 |
| mg/mL BA | | | | | | |
| 0.2 + 1.6 | 92.6 | 93.7 | 97.9 | 99.4 | 88.3 | 90.6 |
| mg/mL BA | | | | | | |
| 0.1 Alone | 108.7 | 91.8 | Not | Not | Not | Not |
| | | | Tested | Tested | Tested | Tested |

From the above ELISA quantitation assay results, DART-A recovery utilizing 0.01% PS80 and 1.6 mg/mL BA is above 88% after 48 hours. These results are within typical variation range of this type of bioassay (50%-150%). In addition, the absence of benzyl alcohol had no effect on the ability of the PS80 to inhibit protein adsorption as recovery is above 91%. These results indicate that levels of 0.05 mg/mL PS80 or 0.1 mg/mL PS80 provides sufficient blocking activity.

### 2.3. Preservative Selection Studies to Enable Single Pump Administration

The Stabilizer 1 solution contains 0.9% BA to act as a preservative. In changing from a two-pump administration to a one-pump administration, the BA concentration is desirably lowered to meet patient safety standards. These standards state that the maximum allowable daily intake of BA desirably should not exceed 5 mg/kg. Using these standards and planning for various clinical scenarios (i.e. low patient weight, keep the vein open flow rate), the target BA concentration for single pump administration was determined as below.
5 mg/kg/day is the maximum allowable BA dose
Assuming 40 kg patient, maximum amount of BA per day: 200 mg
Pump delivery to keep vein open: 5 mL/hr
Continuous infusion over 24 hours is desired→Minimum flow rate is 120 mL/day
Minimum BA concentration in solution is 1.7 mg/mL (200 mg/120 mL) or 0.17%

The selection of stabilizer preservatives was evaluated in a series of studies. FIG. 10 summarizes the different studies performed and each study is described in more detail below.

### 2.4. Preservative Effectiveness of Lower Benzyl Alcohol Concentrations

In this study, five different BA concentrations (listed in Table 15 below) were evaluated to determine the lowest concentration of BA required to inhibit microbial growth over 48 hours.

TABLE 15
Five BA Concentrations Tested and Rationale
Behind Choosing These Concentrations

| | BA Conc. | BA Conc. | |
|---|---|---|---|
| Conc. # | (%) | (mg/mL) | Rationale |
| 1 | 0.90 | 9.0 | Concentration in Stabilizer 1 |
| 2 | 0.24 | 2.4 | Slightly above target concentration to help |
| | | | determine future study stabilizer solutions |
| 3 | 0.17 | 1.7 | Target stabilizer solution concentration |
| 4 | 0.10 | 1.0 | Slightly below target concentration to help |
| | | | determine future study stabilizer solutions |
| 5 | 0.00 | 0.0 | Negative Control |

The single ambulatory pump dose preparation scheme and administration configuration was simulated. For this study saline, PS80, rHA and BA were combined before adding the DART-A DP formulation. Ten vials of reconstituted Pseudomonas aeruginosa (ATCC 9027) were pooled before being aliquoted to create the final formulations. To samples were collected from the mixing bottles upon completion of the formulation preparation steps. Corresponding pump cassettes were filled with the remaining stabilizer solutions and incubated at 32±2° C. The 24-hour and 48-hour time points were thoroughly mixed within the cassettes to ensure a uniform P. aeruginosa suspension before sample collection. After collection, samples were immediately subjected to bioburden testing. The microbial challenge testing results can be seen below in Table 16.

TABLE 16
P. Aeruginosa Colony Count in BA Containing Formulations
Over 48 Hours

| Sample ID | 0 Hours | 24 Hours | 48 Hours |
|---|---|---|---|
| 0.9% BA + PS80 | 0 | 0 | 0 |
| 0.24% BA + PS80 | 20 | 4 | 0 |
| 0.17% BA + PS80 | 18 | TNTC | TNTC |
| 0.0% BA + PS80 | 23 | TNTC | TNTC |
| 0.9% BA + PS80 + rHA | 0 | 0 | — |
| 0.24% BA + PS80 + rHA | 17 | 2 | — |
| 0.17% BA + PS80 + rHA | 17 | TNTC | TNTC |
| 0.0% BA + PS80 + rHA | 18 | TNTC | TNTC |

TNTC: Too numerous to count, represents significant microbial growth

The above results suggest that the effective preservative concentration of BA lies between 0.24% and 0.17% for P. aeruginosa. However, this effective BA level is still above the average daily intake for a 40 kg patient under the proposed single pump administration configuration. The results also show that the presence of rHA in the stabilizer solution does not affect microbial growth and its removal from the Stabilizer 1 solution will not alter the preservative's effectiveness.

### 2.5. Alternative Preservative Evaluation Studies

In this study, three preservatives (BA, meta-cresol and MP) were evaluated by comparing their effect on DART-A stability and aggregation under accelerated stress conditions (40±2° C.). To create the final formulations, each component was combined gravimetrically. The final compositions of the stabilizer solutions are listed below in Table 17.

TABLE 17
Table of Final Stabilizer Solutions Tested Evaluating DART-A
Protein Aggregation in the Presence of Alternative Preservatives

| | | Preservative | DART-A | PS80 | NaCl |
|---|---|---|---|---|---|
| Formulation | Preservative | Concentration | Concentration | Concentration | Concentration |
| # | Name | (%) | (mg/mL) | (mg/mL) | (mg/mL) |
| 1 | BA | 0.24 | 0.1 | 0.1 | 8.8 |
| 2 | Cresol | 0.30 | 0.1 | 0.1 | 8.8 |
| 3 | MP | 0.10 | 0.1 | 0.1 | 8.8 |

Each stabilizer solution was aliquoted into glass vials and incubated at 40±2° C. and analyzed after 0, 1 and 2 weeks. The results of each solution can be seen below in Table 18.

TABLE 18
DART-A Stability and Aggregation Results Within Alternative
Preservatives

| | | Results | Time (Weeks) | | |
|---|---|---|---|---|---|
| Formulation | Test | Reported | 0 | 1 | 2 |
| 0.24% Benzyl | Appearance | | Clear, | Clear, | Clear, |
| Alcohol | | | colorless, free | colorless, | colorless, |
| Containing | | | of particles | few particles | few particles |
| Stabilizer | pH | pH | 6.3 | 6.3 | 6.2 |
| Solution | Osmolality | mOsm | 324 | 326 | 328 |
| | SE-HPLC | % Main | 97.8 | 97.9 | 95.0 |
| | | % HMW | 1.8 | 2.0 | 3.2 |
| | | % LMW | 0.4 | 0.1 | 1.8 |
| | Particle | >2 μm | 1,005 | 4,748 | 10,973 |
| | Count | >5 μm | 180 | 480 | 647 |
| | | >10 μm | 38 | 102 | 85 |
| | | >25 μm | 2 | 8 | 5 |
| 0.1% | Appearance | | Clear, | Clear, | Clear, |
| Methylparaben | | | colorless, free | colorless, | colorless, |
| Containing | | | of particles | few particles | few particles |
| Stabilizer | pH | pH | 6.2 | 6.1 | 6.0 |
| Solution | Osmolality | mOsm | 309 | 308 | 310 |
| | SE-HPLC | % Main | 96.9 | 96.0 | 94.3 |
| | | % HMW | 2.2 | 3.8 | 5.2 |
| | | % LMW | 0.9 | 0.2 | 0.6 |
| | Particle | >2 μm | 323 | 8,644 | 10,401 |
| | Count | >5 μm | 114 | 248 | 347 |
| | | >10 μm | 64 | 64 | 68 |
| | | >25 μm | 11 | 4 | 3 |
| 0.1% Cresol | Appearance | | Cloudy, white, | Clear, | Clear, |
| Containing | | | many particles | colorless, | colorless, |
| Stabilizer | pH | pH | 6.3 | 6.3 | 6.2 |
| Solution | Osmolality | mOsm | 322 | 324 | 326 |
| | SE-HPLC | % Main | 95.7 | 96.5 | 95.6 |
| | | % HMW | 3.3 | 2.4 | 2.8 |
| | | % LMW | 1.0 | 1.2 | 1.6 |
| | Particle | >2 μm | 36,773 | 67,536 | 56,547 |
| | Count | >5 μm | 3,445 | 35,705 | 29,898 |
| | | >10 μm | 5,707 | 7,335 | |
| | | >25 μm | 1 | 13 | 53 |

The above results show that pH and osmolality remain constant while the number of visible particles increase within all three stabilizer solutions after incubation at 40±2° C. for two weeks. However, the cresol-containing solution contains more visible and sub-visible particles compared to solutions containing BA and MP and fails USP specifications due to the number of particles ≥10 μm. In addition, the MP-containing solution showed an increase in DART-A protein aggregation after two weeks that is not observed in the other two solutions. Based on the above studies, combinations containing target levels of BA (≤0.17%) and low levels of MP (≤0.1%) were further evaluated by assessing their effect on DART-A protein aggregation. The solution combinations were created in a manner similar to the single agent study where each component was combined gravimetrically. The final compositions of the combinations tested are listed below in Table 19.

TABLE 19
Table of Final Alternative Preservative Combinations Tested to
Evaluate DART-A Protein Stability

| | Benzyl Alcohol | Methylparaben | DART-A | | |
|---|---|---|---|---|---|
| | Concentration | Concentration | Concentration | PS80 Concentration | NaCl Concentration |
| # | (mg/mL) | (mg/mL) | (μg/mL) | (mg/mL) | (mg/mL) |
| 1 | 1.7 | 0.5 | 0.4 | 0.1 | 8.8 |
| 2 | 1.7 | 0.1 | 0.4 | 0.1 | 8.8 |
| 3 | 1.7 | 0.0 | 0.4 | 0.1 | 8.8 |
| 4 | 1.2 | 0.5 | 0.4 | 0.1 | 8.8 |
| 5 | 1.2 | 0.1 | 0.4 | 0.1 | 8.8 |
| 6 | 1.2 | 0.0 | 0.4 | 0.1 | 8.8 |

All solutions were staged at 5±3° C. and 25±2° C. and analyzed at 0, 1, 3, 5, and 7 days. The results of each solution can be seen below in Table 20 and Table 21.

TABLE 20
DART-A Protein Stability in BA and MP Containing Stabilizer
Solutions at 5 ± 3° C.

| Analysis | T₀ | Day 7 | T₀ | Day 7 | T₀ | Day 7 |
|---|---|---|---|---|---|---|
| pH | 6.0 | 6.2 | 6.0 | 6.2 | 6.0 | 6.2 |
| | 0.17% BA + | | 0.17% BA + | | 0.17% BA + | |
| | 0.05% MP | | 0.01% MP | | 0.0% MP | |
| Osmolality (mOsm) | 327 | 326 | 327 | 324 | 324 | 324 |
| HIAC ≥ 2 μm | 57 | 128 | 53 | 168 | 93 | 158 |
| HIAC ≥ 5 μm | 25 | 37 | 22 | 50 | 40 | 42 |
| HIAC ≥ 10 μm | 10 | 22 | 10 | 15 | 20 | 3 |
| HIAC ≥ 25 μm | 3 | 2 | 2 | 2 | 3 | 0 |
| SE-HPLC (% HMW) | IR | 1.5 | IR | 0.8 | IR | 1.1 |
| SE-HPLC (% LMW) | 0.0 | 0.0 | 0.0 | 0.0 | 0.0 | 0.0 |
| Appearance** | CCN | CCF | CCN | CCF | CCN | CCF |
| | 0.12% BA + | | 0.12% BA + | | 0.12% BA + | |
| | 0.05% MP | | 0.01% MP | | 0.0% MP | |
| Osmolality (mOsm) | 323 | 323 | 320 | 319 | 318 | 318 |
| HIAC ≥ 2 μm | 152 | 177 | 85 | 193 | 83 | 308 |
| HIAC ≥ 5 μm | 50 | 52 | 37 | 68 | 30 | 103 |
| HIAC ≥ 10 μm | 27 | 12 | 13 | 23 | 10 | 25 |
| HIAC ≥ 25 μm | 2 | 2 | 0 | 2 | 0 | 5 |
| SE-HPLC (% HMW) | IR | 1.7 | IR | 1.8 | IR | 1.7 |
| SE-HPLC (% LMW) | 0.0 | 0.0 | 0.0 | 0.0 | 0.0 | 0.0 |
| Appearance** | CCV | CCF | CCN | CCF | CCV | CCF |

**CCN: clear, colorless, no visible particles CCV: clear, colorless, very few visible particles;
CCF: clear, colorless, few visible particles;
IR: Invalid Results

TABLE 21
DART-A Protein Stability in BA and MP Containing Stabilizer
Solutions at 25 ± 2° C.

| Analysis | T₀ | Day 7 | T₀ | Day 7 | T₀ | Day 7 |
|---|---|---|---|---|---|---|
| pH | 6.0 | 6.2 | 6.0 | 6.2 | 6.0 | 6.2 |
| | 0.17% BA + | | 0.17% BA + | | 0.17% BA + | |
| | 0.05% MP | | 0.01% MP | | 0.0% MP | |
| Osmolality (mOsm) | 327 | 327 | 327 | 326 | 324 | 325 |
| HIAC ≥ 2 μm | 57 | 98 | 53 | 90 | 93 | 82 |
| HIAC ≥ 5 μm | 25 | 30 | 22 | 38 | 40 | 32 |
| HIAC ≥ 10 μm | 10 | 7 | 10 | 18 | 20 | 7 |
| HIAC ≥ 25 μm | 3 | 0 | 2 | 3 | 3 | 0 |
| SE-HPLC (% HMW) | 12.8 | 2.4 | 13.0 | 2.1 | 12.3 | 2.2 |
| SE-HPLC (% LMW) | 0.0 | 0.1 | 0.0 | 0.1 | 0.0 | 0.2 |
| Appearance** | CCN | CCF | CCN | CCF | CCN | CCF |
| | 0.12% BA + | | 0.12% BA + | | 0.12% BA + | |
| | 0.05% MP | | 0.01% MP | | 0.0% MP | |
| Osmolality (mOsm) | 323 | 323 | 320 | 319 | 318 | 320 |
| HIAC ≥ 2 μm | 152 | 82 | 85 | 88 | 83 | 98 |
| HIAC ≥ 5 μm | 50 | 23 | 37 | 28 | 30 | 35 |
| HIAC ≥ 10 μm | 27 | 10 | 13 | 7 | 10 | 10 |
| HIAC ≥ 25 μm | 2 | 0 | 0 | 0 | 0 | 3 |
| SE-HPLC (% HMW) | 13.1 | 2.3 | 12.5 | 2.2 | 11.7 | 2.0 |
| SE-HPLC (% LMW) | 0.0 | 0.2 | 0.0 | 0.1 | 0.0 | 0.1 |
| Appearance** | CCV | CCM | CCN | CCM | CCV | CCF |

**CCN: clear, colorless, no visible particles CCV: clear, colorless, very few visible particles;
CCF: clear, colorless, few visible particles;
CCM: clear, colorless, many visible particles

The above results indicate that no significant changes in pH or osmolality were observed across all stabilizer solutions. In addition, all stabilizer solutions met the USP specification for sub-visible particle counts. Few visible particles were observed in all stabilizer solutions after seven days. Though the initial levels of HMW species were not available due to invalid analytical test results, the levels of HMW species after 7 days were acceptable in all solutions. Therefore, from a product quality standpoint all these solution conditions were considered acceptable.

### 2.6. Microbial Challenge Testing Evaluating the Combination of Benzyl Alcohol and Methylparaben as a Preservative

Microbial challenge testing was performed to evaluate the preservative effect of stabilizer solutions containing ≤0.17% BA and ≤0.1% MP. Microbial challenge testing was chosen as it more accurately represents the proposed dose preparation and dilution scheme where doses will be prepared aseptically and the number of microbes that may be introduced to the solution would be low. Microbial challenge testing was performed in two stages. The first stage determined the effective concentrations of BA and MP while the second stage tested the final formulation according to USP specifications. Each stage is described in detail below.

### 2.7. Determination of Effective Benzyl Alcohol and Methylparaben Concentrations

In this study, combinations of BA and MP (listed in Table 22 below) were evaluated to determine the effective concentration of BA and MP required to inhibit microbial growth over 168 hours. These combinations were tested in both P. aeruginosa (bacteria) and C. albicans (fungi) to compare preservative effectiveness across organisms.

TABLE 22
Seven Combinations Tested and Rationale Behind Choosing These
Concentrations

| | Concentration (%) | | |
|---|---|---|---|
| Combination | BA | MP | Rationale |
| 1 | 0.90 | 0.00 | Dose is known to be |
| | | | effective—negative control |
| 2 | 0.24 | 0.00 | Lowest concentration of BA that |
| | | | was previously shown to be |
| | | | effective against P. Aeruginosa |
| 3 | 0.20 | 0.00 | Slightly above target BA |
| | | | concentration to help determine |
| | | | future administration options |
| 4 | 0.17 | 0.00 | Target BA stabilizer solution |
| | | | concentration |
| 5 | 0.17 | 0.05 | Target BA stabilizer solution |
| | | | concentration |
| | | | Target MP concentration. |
| 6 | 0.17 | 0.01 | Target BA stabilizer |
| | | | solution concentration |
| | | | MP concentration below target |
| | | | to limit protein aggregation |
| 7 | 0.12 | 0.05 | Slightly below target BA |
| | | | concentration so |
| | | | patient ADI limits are |
| | | | not reached Target |
| | | | MP concentration |
| 8 | 0.00 | 0.00 | Positive control |

For the study, the single ambulatory pump dose preparation scheme was simulated. P. aeruginosa and C. albicans microbes were reconstituted and pooled before being added to the final dosing solutions. Each solution was added to its corresponding cassette and To samples were collected immediately upon cassette filling. This procedure accounted for any changes in microbial growth patterns that may be associated with syringe stress exposure. After To collection, corresponding cassettes were incubated at 32±2° C. The 24-, 72- and 168-hour time points were thoroughly mixed to ensure a uniform microbe suspension before sample collection. After collection, samples were immediately subjected to microbial challenge testing. The microbial challenge testing results can be seen below in Table 23 and Table 24.

TABLE 23
P. Aeruginosa Colony Count in BA and MP Containing Stabilizer
Solutions Over 168 Hours

| Sample ID | 0 Hours | 24 Hours | 72 Hours | 168 Hours |
|---|---|---|---|---|
| 0.9% BA | 0 | 0 | 0 | 0 |
| 0.24% BA | 3 | 7 | 15 | TNTC |
| 0.20% BA | 3 | 10 | NA | Overgrown |
| 0.17% BA | 5 | 196 | NA | Overgrown |
| 0.17% BA + 0.05% MP | 5 | 5 | NA | Overgrown |
| 0.17% BA + 0.01% MP | 3 | 2 | NA | Overgrown |
| 012% BA + 0.05% MP | 4 | 0 | 1 | 0 |
| 0% BA + 0% MP | 2 | Overgrown | Overgrown | Overgrown |

TNTC: Too numerous to count, represents significant microbial growth

TABLE 24
C. Albicans Colony Count in BA and MP Containing Stabilizer
Solutions Over 168 Hours

| Sample ID | 0 Hours | 24 Hours | 72 Hours | 168 Hours |
|---|---|---|---|---|
| 0.9% BA | 16 | 0 | 0 | 0 |
| 0.17% BA | 23 | >50 | >50 | >2,500 |
| 0.17% BA + 0.05% MP | 22 | 14 | >50 | >2,500 |
| 0.17% BA + 0.01% MP | 22 | >50 | >50 | >2,500 |
| 012% BA + 0.05% MP | 19 | 17 | >50 | >2,500 |
| 0% BA + 0% MP | 23 | >50 | >50 | >2,500 |

Plates reported as “>50” were observed to be TNTC (Too Numerous to Count), from a 5 mL plated sample volume
Plates reported as “>2,500” were observed to be TNTC from a 0.1 mL plated sample volume
TNTC: Too numerous to count, represents significant microbial growth

The results from Table 23 and Table 24 showed that the BA concentration in the Stabilizer 1 solution (0.9%) is both bactericidal and fungicidal while the single pump configuration target BA concentration (0.17%) alone does not inhibit growth. The above results also show that the combination of MP and BA increases the antimicrobial properties of the stabilizer solution compared to BA alone. However, the bacteriostatic and fungistatic properties of the 0.17% BA+0.05% MP combination were only observed over 24 hours. Since the BA concentration in the stabilizer solution is limited due to patient safety concerns, to increase the preservative effectiveness duration, the MP concentration in the stabilizer solution could be increased to 0.1%. However, when the concentrated stabilizer solution is presented as a 20 mL additive volume, this level of MP leads to high solution pH (≥9.0), solution turbidity, and PS80 immiscibility. To address these issues, a series of stabilizer solution optimizing studies were performed to determine the final concentrated stabilizer solution.

The first study explored lower levels of PS80 in the concentrated stabilizer solution to determine if the turbidity and solution instability or immiscibility issues can be addressed. The results showed that DART-A protein recovery was significantly diminished in final dosing solutions that contain PS80 concentrations below 0.003%. However, this concentration of PS80 remains turbid at room temperature. A subsequent study evaluating larger concentrated stabilizer solution additive volume presentations was performed to determine if lowering the levels of all excipients in the concentrated stabilizer solution helps address the high turbidity issue. Results of this study showed that a 40 mL additive stabilizer volume with final levels of PS80 above 0.003% did not form droplets upon storage and was only slightly opalescent. To determine optimal concentrated stabilizer solution pH, studies varying the formulation buffer strength were performed. The first study showed that increasing the sodium phosphate levels in the concentrated stabilizer solution effectively lowers solution pH. However, below pH 8.4, the target level of MP does not remain in solution. As a result, variable concentrations of MP were evaluated in a subsequent study. These results showed that 30-50% of the target MP concentration stays in solution at pH≤8.0. Based on the results of the previous PS80 and MP studies, a final study was performed evaluating variable concentrations of MP and PS80 to determine the optimal combination that addresses solution turbidity at an acceptable pH. The results of this study showed that a concentrated Stabilizer 2 solution, composed of 20 mM sodium phosphate, 13.2 mg/mL BA, 4.25 mg/mL MP and 0.25 mg/mL PS80 is acceptable and only slightly opalescent upon storage with a final solution pH of 8.2. When 40 mL of this stabilizer solution is diluted in a 250 mL saline bag, the resulting dosing solution will contain 0.17% BA and 0.055% MP. This combination previously showed bacteriostatic and fungistatic effects for only 24 hours. However, the antimicrobial effects of MP are diminished in high pH environments and the stabilizer solution re-formulation significantly lowered the pH of the final dosing solution. As a result, the preservative effectiveness of the re-formulated Stabilizer 2 solution having final preservative levels of 0.17% BA and 0.05% MP in the dosing solution, was examined through microbial challenge testing of USP recommended microbes.

### 2.8. Microbial Challenge Testing to Define Final Preservative Concentrations

To meet current USP standards for preservatives, no growth must be observed over the desired administration time when the formulation is tested against certain USP recommended microbes. Based on previous studies, C. albicans and A. brasiliensis microbes were the most resistant to the preservatives in the dosing solution; as a result, only these two microbes were tested. Only the 0.17% BA+0.05% MP stabilizer solution was tested and compared to a no preservative control. Microbial growth was monitored over 72 hours with sampling time points at 0, 48, and 72 hours. Dose preparation, setup and methods were carried out in a similar manner to previous microbial challenge tests performed. The results of this study can be seen below in Table 25.

TABLE 25
Microbial Challenge Results of 0.17 BA + 0.05% MP Stabilizer
Solution for All USP Recommended Microbes

| | | 0 Hours | | 48 Hours | | 72 Hours | |
|---|---|---|---|---|---|---|---|
| | Dosing | Plate | Plate | Plate | Plate | Plate | Plate |
| Microbe | Solution | 1 | 2 | 1 | 2 | 1 | 2 |
| C. Albicans | 0.0% BA + | 85 | 87 | >250 | >250 | >250 | >250 |
| | 0.0% MP | | | | | | |
| C. Albicans | 0.17% BA + | 46 | 35 | 11 | 10 | 15 | 10 |
| | 0.05% MP | | | | | | |
| A. Brasiliensis | 0.17% BA + | 46 | 35 | 11 | 10 | 15 | 10 |
| | 0.05% MP | | | | | | |

From the results in Table 25, the combination of 0.17% BA+0.05% MP in the dosing solution is fungistatic against both A. brasiliensis and C. albicans as the 48-hour and 72-hour counts are not 0.5 log greater than 0-hour count. As a result, Stabilizer 2, which provides preservative concentrations of 0.17% BA and 0.05% MP in the final dosing solution, will enable single pump administration of DART-A over 48 hours.

### 2.9. Confirmatory Microbial Challenge Testing of all Five USP Recommended Microbes

To confirm the anti-microbial properties of Stabilizer 2 once diluted in saline (i.e., in the dosing solution), microbial growth testing was performed by SGS Life Sciences. The USP recommended microbes required for testing are listed below in Table 26.

TABLE 26
Five USP Recommended Microbes for Microbial
Challenge Testing

| | | ATCC |
|---|---|---|
| Name | Strain | Number |
| Pseudomonas | Bacteria | 9027 |
| Aeruginosa | (Gram Negative) | |
| Staphylococcus | Bacteria | 6538 |
| Aureus | (Gram Positive) | |
| Escherichia Colt | Bacteria | 8739 |
| | (Gram Negative) | |
| Candida Albicans | Fungi | 10231 |
| Aspergillus | Fungi | 16404 |
| Brasiliensis | | |

A high dosing solution (150 kg patient at 1,000 ng/kg cohort) was prepared to represent the maximum preservative dilution. In addition, the target concentration of BA and MP was adjusted to lower preservative concentrations of 0.157% and 0.05% to demonstrate preservative effectiveness at slightly lower levels. Microbial growth studies were performed on all five USP recommended microbes with a target concentration between 10 and 100 CFU/mL. The microbial growth results are listed below in Table 27.

TABLE 27
Microbial Growth Results of Five USP Recommended Microbes
Using Single Pump Compatibility Dose Preparation Scheme

| Organism | Quantification | 0 Hours | 48 Hours | 72 Hours | 120 Hours |
|---|---|---|---|---|---|
| P. | CFU/g | 650 | <100 | <100 | <100 |
| Aeruginosa | Log(CFU/g) | 2.81 | <2.00 | <2.00 | <2.00 |
| E. Coli | CFU/g | 1000 | <100 | <100 | <100 |
| | Log(CFU/g) | 3.00 | <2.00 | <2.00 | <2.00 |
| S. Aureus . | CFU/g | 400 | <100 | <100 | <100 |
| | Log(CFU/g) | 2.60 | <2.00 | <2.00 | <2.00 |
| C. Albicans | CFU/g | 450 | <100 | <100 | <100 |
| | Log(CFU/g) | 2.65 | <2.00 | <2.00 | <2.00 |
| A. | CFU/g | 850 | <100 | <100 | <100 |
| Brasiliensis | Log(CFU/g) | 2.93 | <2.00 | <2.00 | <2.00 |

Based on the above results, the dilution of Stabilizer 2 slightly below the intended target concentration meets the criteria for no growth over 120 hours as the log growth values for each microbe do not increase by a value >0.5 log compared to the initial concentration of microbe. These studies demonstrate that Stabilizer 2 diluted using the dose preparation scheme designed for single pump administration successfully limits microbial growth over a period of 120 hours.

### 2.10. Allowable Ranges of Excipients in the Final Stabilizer 2 Solution

To facilitate patient compliance and comfort, a four-day continuous administration of the formulation from the IV bag or cassettes after dose preparation can be implemented. Transitioning to four-day administration will require the KVO rate be decreased to 2.5 mL/hr which allows for more preservative to be in solution without reaching acceptable daily intake (ADI) levels. ADI values based on administration from 250 mL bag over 4 days will allow for the following upper limits of allowable excipient range in the stabilizer solution.
Upper BA Limit: 25.8 mg/mL
Upper MP Limit: 51.7 mg/mL

However, BA levels above 15.5 mg/mL result in solution instability or immiscibility and MP levels above 5.3 mg/mL produce a final solution above pH 8.4. Therefore, the proposed allowable range for BA and MP in the final Stabilizer 2 solution are as follows:
BA Target Concentration: 13.2 mg/mL (Allowable Range: 11.6 to 15.5 mg/mL)
MP Target Concentration: 4.25 mg/mL (Allowable Range: 3.9 to 5.3 mg/mL)

Since transitioning to four-day administration involves lowering the KVO rate, the minimum anticipated DART-A dosing solution concentration (corresponding to dosing a 40 kg patient at a dose of 30 ng/kg) must increase from 10 to 20 ng/mL. Protein recovery studies (evaluated by ELISA (rhIL3Rα) simulating clinical dose preparation and administration at this 20 ng/mL protein concentration were performed with lower than target (0.25 mg/mL) concentrations of PS80. The results of these studies are summarized below in Table 28.

TABLE 28
DART-A Recovery After Storage and Administration

| | | Target | | |
|---|---|---|---|---|
| Final PS80 | | diabody | Actual | % |
| Concentration | Time Point | Concentration | Concentration | Recovery |
| 0.1 mg/mL | T₀ | 20 ng/mL | 19.9 ng/mL | — |
| | Post 24 Hr | | | |
| | Storage and | 20 ng/mL | 19.3 ng/mL | 97.0 |
| | 96 Hr | | | |
| | Admin | | | |
| 0.12 mg/mL | T₀ | 20 ng/mL | 21.6 ng/mL | — |
| | Post 24 Hr | | | |
| | Storage and | 20 ng/mL | 20.6 ng/mL | 95.4 |
| | 96 Hr | | | |
| | Admin | | | |

Protein recovery studies show full recovery of this higher 20 ng/mL dosing solution after 24 hours of storage and 96 hours of administration when utilizing a 0.10 mg/mL PS80 stabilizer formulation. Four-day administration will only allow for a slight change in the upper PS80 specification as this limit is driven by solution appearance (i.e., miscibility). Assuming a 250 mL saline bag is used for dose preparation, the proposed allowable range for PS80 in the final stabilizer solution is as follows:
PS80 Target Concentration: 0.25 mg/mL (Allowable Range: 0.10 to 0.35 mg/mL)

### 2.11. Conclusions

The above studies support a DART-A DP formulation with a final composition of 0.1 mg/mL in 10 mM sodium phosphate, 150 mM sodium chloride, 0.1 mg/mL PS80, pH 6.0. Such DART-A DP formulation may be a 5 mL fill in a 5 cc vial.

The above studies further support a first stabilizer solution, Stabilizer 1, composed of 10 mM sodium phosphate, 150 mM sodium chloride, 0.1 mg/mL PS80, 9.0 mg/mL BA and 0.1 mg/mL rHA at pH 6.0, to be combined with the DART-A DP formulation for intravenous administration using two syringe pumps or two ambulatory pumps. Stabilizer 1 is particularly useful for use in pediatric patients, patients with low body weight, and/or patients that require a higher IV flow rate (e.g., greater than about 5 mL/hr). Stabilizer 1, composed of 10 mM sodium phosphate, 150 mM sodium chloride, and 0.1 mg/mL PS80, at pH 6.0, and lacking BA is particularly preferred for use in pediatric patients. Such Stabilizer 1 solution lacking BA, is particularly suitable for administration within 24 hours after being combined with the DART-A DP formulation.

Multiple changes to Stabilizer 1 were required to support single ambulatory pump administration. The first change involved removing the current blocking agent rHA. The second formulation change involves the BA preservative, which is too concentrated in Stabilizer 1 for single ambulatory pump administration. Based on the studies described above, a second stabilizer solution, Stabilizer 2, composed of 20 mM sodium phosphate, 13.2 mg/mL BA, 4.25 mg/mL MP and 0.25 mg/mL PS80 at pH 8.2 will support single pump administration. When diluted in a 250 mL saline bag (having a nominal volume of 270 mL), the resulting dosing solution contains 0.03 mg/mL PS80, 1.7 mg/mL BA and 0.55 mg/mL MP. The DART-A DP formulation is subsequently added to create the final dosing solution and is suitable for four days of continuous IV administration using a single ambulatory pump.

### Example 3

### DART-A Dual-Pump Ambulatory Infusion Compatibility Study Utilizing Stabilizer 1 Solution

This example summarizes the compatibility of the DART-A DP formulation (diluted in Stabilizer 1) with the infusion components and the DART-A recovery in a dual pump ambulatory infusion configuration. The infusion configuration uses two ambulatory pumps with pump 1 delivering the DART-A DP formulation (diluted in Stabilizer 1) and pump 2 delivering saline to maintain a combined flow rate of at least 10 mL/hr.

The objectives of the study were:
(A) To evaluate the compatibility of the DART-A DP formulation, diluted in Stabilizer 1, with the infusion components and the overall DART-A recovery during continuous infusion at room temperature for 24, 48, and 72 hours;
(B) To test BA content of Stabilizer 1 and evaluate the stability of DART-A in the medication cassette under shaking at room temperature for 24, 48, and 72 hours; and
(C) To evaluate the stability of DART-A in the medication cassette under shaking at elevated temperature (37° C.) for 72 hours.

### 3.1. Study Design

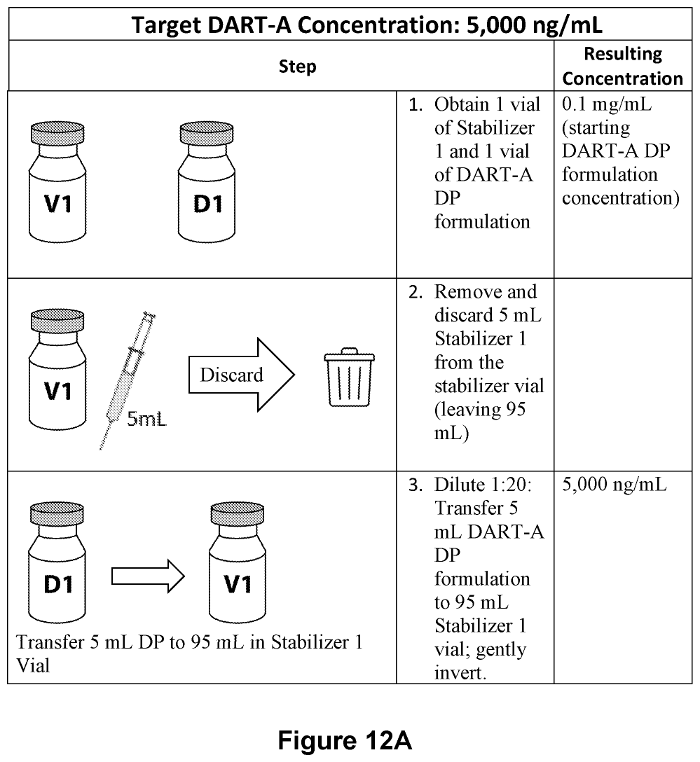

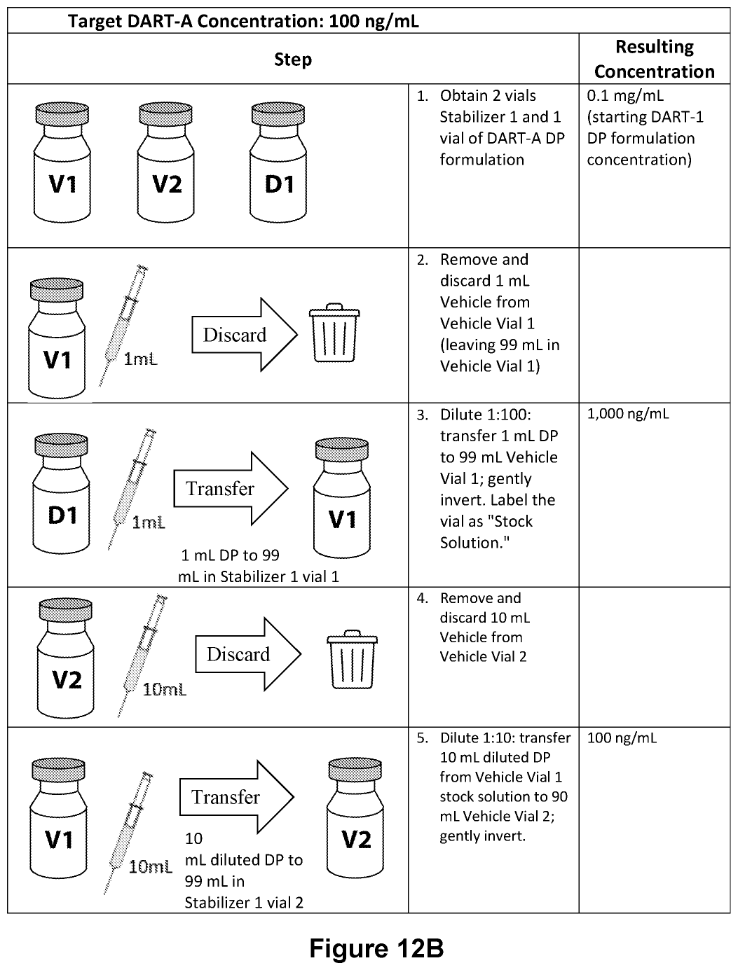

An infusion configuration using two ambulatory pumps was designed for continuous administration of the DART-A DP dosing solution. As shown in FIG. 11, the DART-A DP formulation (diluted in Stabilizer 1) was loaded in the medication cassette of ambulatory pump 1 and flowed through the administration set (tubing) at a flow rate of about 1 mL/hr or less into port B of the 3-way stopcock. Saline solution (0.9% sodium chloride injection USP) was loaded in the medication cassette of ambulatory pump 2 and flowed through the administration set (tubing) at 5 mL/hr into port A of the 3-way stopcock. The DART-A DP formulation and saline mixed and flowed through an extension set with a 0.2 μm in-line filter into the Central Venous Catheter (CVC) for administration into the subject. This 2-pump infusion configuration is necessary because the infusion rate of the DART-A DP dosing solution is at 1 mL/hr or less and the recommended flow rate is >10 mL/hr to keep the CVC port open without any blood clotting. Pump 2 delivers saline at 10 mL/hr to maintain the combined flow rate of at least 10 mL/hr. Dosing solutions to be loaded onto the medication cassettes were prepared by diluting the DART-A DP formulation in Stabilizer 1 as shown in FIGS. 12A-12B.

This study used a bracketed approach to cover a wide dose range by testing doses of 30 ng/kg/day to 1000 ng/kg/day. Table 29 shows the dose calculations used for a subject of 80 kg body weight as an example to determine the dose concentration, dose volume and the flow rate. The dose calculations for other subjects can be calculated similarly using their corresponding body weights.

TABLE 29
Dose Preparation Table Illustrating the Calculations Of
DART-A For Administration To A Subject Of 80 Kg Body Weight

| | Dose | | | | |
|---|---|---|---|---|---|
| | DART- | Total Dose | DART-A | Dose | |
| Subject Body | A/kg/day | DART- | Dose Conc. | volume/ | Flow rate |
| Weight (kg) | (ng) | A/day (ng) | (ng/mL) | day (mL) | (mL/hr) |
| 80 | 30 | 2400 | 100 | 24 | 1.0 |
| 80 | 100 | 8000 | 500 | 16 | 0.7 |
| 80 | 300 | 24000 | 1000 | 24.0 | 1.0 |
| 80 | 500 | 40000 | 2000 | 20.0 | 0.8 |
| 80 | 700 | 56000 | 2000 | 28.0 | 1.2 |
| 80 | 900 | 72000 | 4000 | 18.0 | 0.8 |
| 80 | 1000 | 80000 | 5000 | 16.0 | 0.7 |

### 3.2. Sampling Plan

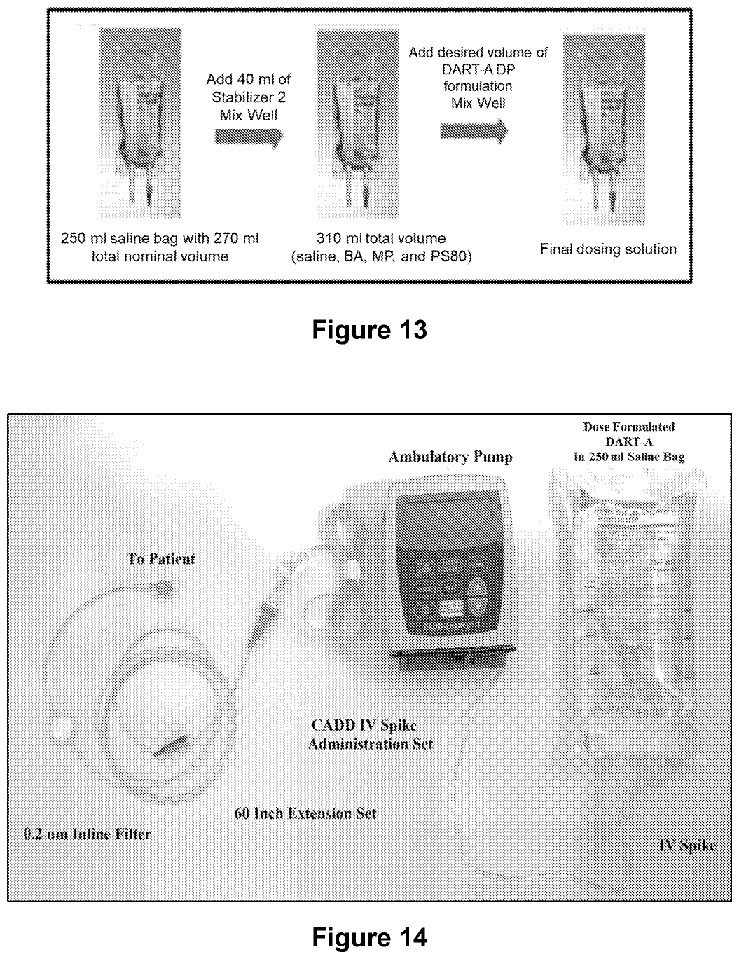

Three studies were carried out to evaluate the compatibility of DART-A DP formulation with the infusion components, drug recovery and the stability of the DART-A loaded in the medication cassette during infusion at room temperature (22° C.±2° C.), and at an elevated temperature (37° C.) under shaking for up to 72 hours. FIG. 13 describes the five (5) sampling points (#1-#5) used in these studies.

### 3.2.1. Study 1

Study 1 evaluated the compatibility and the overall DART-A recovery at the point of entry, sampling point #5, and during continuous infusion at room temperature (22° C.±2° C.) for 24, 48, and 72 hours.

### 3.2.2. Study 2

The DART-A DP formulation was diluted in Stabilizer 1 to prepare the DART-A dosing solution. Stabilizer 1 contains 0.9% BA as the antimicrobial preservative. Study 2 tested the BA content and evaluated the stability of the DART-A in the medication cassette at room temperature (22° C.±2° C.) under shaking (100 rpm) for 24, 48, and 72 hours. Stability of the DART-A was measured using SE-HPLC. Since the highest DART-A DP formulation dose (5000 ng/mL) had the most diluted preservative concentration in the stabilizer and represents the worst-case scenario, only the highest dose samples were tested for BA concentration. Samples were collected at sampling point #2 as shown in FIG. 11.

### 3.2.3. Study 3

Study 3 evaluated the stability of the diluted DART-A in the medication cassette at 37° C. under shaking (100 rpm) for up to 72 hours. Samples were collected at sampling point #2, as shown in FIG. 11, at the 72-hour time point.

### 3.3. Results and Discussion

The results of the compatibility studies above are summarized in Table 30 below.

TABLE 30
Study 1—Ambulatory Infusion Compatibility Study—DART-A
Recovery

| | | DART-A | | DART-A | DART-A |
|---|---|---|---|---|---|
| | | Concentration | DART-A | Monomer | Recovery |
| | | (ng/mL) | Recovery | Peak Area | based on |
| | | derived from | based on | in SE-HPLC | Monomer |
| Target | Dose | quantitation | assay derived | FLR | Peak Area in |
| Concentration | Samples | assay | concentration | (EU * min) | SE-HPLC |
| Low | Expected or | 10.6 | NA | 46006 | NA |
| Concentration | Control (T₀^{A}) | | | | |
| (100 ng/mL) | T = 24 hours | 6.7 | 63.2% | 37026 | 80.5% |
| | T = 48 hours | 12.9 | 121.7% | 44708 | 97.2% |
| | T = 72 hours | 11.0 | 103.8% | 49122 | 106.8% |
| High | Expected or | 313.7 | NA | 1814623 | NA |
| Concentration | Control (T₀^{A}) | 235.2 | 75.0% | 1483536 | 81.8% |
| (5000 ng/mL) | T =24 hours | | | | |
| | T = 48 hours | 294.3 | 93.8% | 1792023 | 98.8% |
| | T =72 hours | 254.4 | 81.1% | 1804667 | 99.5% |

^{A}T₀ = Time (T) = 0

The low concentration as prepared in the study was determined by DART-A quantitation assay (evaluated by ELISA (rhIL3Rα) to be 116.7 ng/mL. As shown in FIG. 11 and Table 29, DART-A infuses at 1.0 mL/hr and gets diluted by saline that infuses at 10 mL/hr. The expected DART-A concentration at To is 116.7 ng/mL×1.0 mL/hr÷(1.0 mL/hr+10 mL/hr)=10.6 ng/mL.

The high concentration as prepared in the study was determined by DART-A quantitation assay (evaluated by ELISA (rhIL3Rα) to be 5067.5 ng/mL. As shown in FIG. 11 and Table 29, DART-A infuses at 0.66 mL/hr and gets diluted by saline that infuses at 10 mL/hr. The expected DART-A concentration at To is 5067.5 ng/mL×0.66 mL/hr÷(0.66 mL/hr+10 mL/hr)=313.7 ng/mL.

For both low and high concentration infusions, samples were collected at 24, 48, and 72 hours from sampling point #5. The DART-A quantitation assay, as above, was used to determine DART-A concentration and SE-HPLC with fluorescence detector (FLR SE-HPLC) was used to analyze DART-A monomer peak area. The data was calculated against the expected TO sample to obtain the DART-A recovery. As shown in Table 30, DART-A recovery based on quantitation assay was between 63.2% and 121.7% for the low dose, and between 75.0% and 93.8% for the high dose. Similar to other binding ELISA assays, these ranges are within the typical variation of this type of bioassay (50%-150%). DART-A recoveries for these same samples based on SE-HPLC monomer peak areas were between 80.5% and 106.8% for the low dose, and between 81.8% and 99.5% for the high dose.

Samples collected at 24, 48, and 72 hours from sampling point #5 in Study 1 were also subjected to particle count testing. As shown in Table 31, the particle counts for all samples were low and met USP specifications.

TABLE 31
Study 1—Ambulatory Infusion Compatibility Study—Particle
Counts

| | | Particle | Particle |
|---|---|---|---|
| Target | | Counts/mL | Counts/mL |
| Concentration | Dose Samples | ≥10 μm | ≥25 μm |
| Low Concentration | Saline control | 1 | 0 |
| (100 ng/mL) | T = 0 | 5 | 0 |
| | T = 24 hours | 4 | 1 |
| | T = 48 hours | 7 | 0 |
| | T = 72 hours | 1 | 0 |
| High | Saline control | 1 | 0 |
| Concentration | T = 0 | 14 | 1 |
| (5000 ng/mL) | T = 24 hours | 2 | 1 |
| | T = 48 hours | 0 | 0 |
| | T = 72 hours | 0 | 0 |

Samples collected at 24, 48, and 72 hours from sampling point #2 in Study 2 were analyzed by DART-A quantitation assay and SE-HPLC with fluorescence detector. As shown in Table 32, all samples remained active as demonstrated by DART-A's intact binding activity in the quantitation assay and were stable as shown by minimal decrease of monomer peak area in SE-HPLC assay. DART-A recoveries assessed by both assays were between 91.1% and 110.8%.

TABLE 32
Study 2—Stability and Recovery Of DART-A Loaded In Medication
Cassette At 25° C. Incubation Under 100 Rpm Shaking For Up To 72 Hours

| | | | | DART-A Monomer | |
|---|---|---|---|---|---|
| | | DART-A | DART-A | Peak Area | DART-A Recovery |
| | | Concentration | Recovery | in SE- | based on |
| | | (ng/mL) | based on | HPLC | Monomer |
| Target | Dose | derived from | assay-derived | FLR | Peak Area |
| Concentration | Samples | quantitation assay | concentration | (EU * min) | in SE-HPLC |
| Low | T = 0 | 110.1 | NA | 355660 | NA |
| Concentration | T = 24 | 115.5 | 104.9% | 379757 | 106.8% |
| (100 ng/mL) | hours | | | | |
| | T = 48 | 103.8 | 94.3% | 377121 | 106.0% |
| | hours | | | | |
| | T = 72 | 106.2 | 96.5% | 394028 | 110.8% |
| | hours | | | | |
| High | T = 0 | 5398.7 | NA | 41031471 | NA |
| Concentration | T = 24 | 5241.2 | 97.1% | 40529514 | 98.8% |
| (5000 ng/mL) | hours | | | | |
| | T = 48 | 4967.5 | 92.0% | 41438367 | 101.0% |
| | hours | | | | |
| | T = 72 | 4916.7 | 91.1% | 41582544 | 101.3% |
| | hours | | | | |

Samples collected at 24, 48, and 72 hours from sampling point #2 of the high concentration conditions in Study 2 were also subjected to BA content analysis. As shown in Table 33, BA content dropped slightly possibly due to evaporation and/or adsorption but remained at least 0.75% even after 25° C. incubation under 100 rpm shaking for up to 72 hours. This BA concentration is above the preservative effectiveness concentration tested in a previous study, in which 0.61% of BA concentration provided sufficient anti-microbial activity.

TABLE 33
Study 2—Benzyl Alcohol content in the diluted DART-A DP
formultaion loaded in medication cassette at 25° C. incubation under
100 rpm shaking for up to 72 hours

| | | Expected | Tested | |
|---|---|---|---|---|
| | | Benzyl | Benzyl | |
| | | Alcohol | Alcohol | Benzyl |
| Target | Dose | Concentration | Concentration | Alcohol |
| Concentration | Samples | (%) | (%) | Recovery |
| Low | T = 0 | 0.86% | 0.91% | NA |
| Concentration | T = 24 hours | 0.86% | 0.78% | 90.7% |
| (5000 ng/mL) | T = 48 hours | 0.86% | 0.76% | 88.4% |
| | T = 72 hours | 0.86% | 0.78% | 87.2% |

Samples collected in Study 3 were subjected to analysis by DART-A quantitation assay and SE-HPLC with fluorescence detection. As shown in Table 34, after 37° C. incubation under 100 rpm shaking for up to 72 hours, samples remained active as demonstrated by the DART-A's intact binding activity in the quantitation assay and were stable as evidenced by minimal decrease of monomer peak area in SE-HPLC assay. DART-A recoveries assessed by both assays were between 82.0% and 95.1%.

TABLE 34
Study 3—Stability and Recovery Of DART-A loaded In Medication
cassette at 37° C. incubation under 100 rpm shaking for up to 72 Hours

| | | DART-A | | | |
|---|---|---|---|---|---|
| | | Concentration | DART-A | DART-A Monomer | DART-A Recovery |
| | | (ng/mL) | Recovery | Peak Area | based on |
| | | derived from | based on | in SE-HPLC | Monomer |
| Target | Dose | quantitation | assay-derived | FLR | Peak Area |
| Concentration | Samples | assay | concentration | (EU * min) | in SE-HPLC |
| Low | T = 0 | 111.4 | NA | 397095 | NA |
| Concentration | T = 72 | 99.4 | 89.2% | 368950 | 92.9% |
| (100 ng/mL) | hours | | | | |
| | T = 0 | 5682.9 | NA | 41031489 | NA |
| | T = 72 | 4661.6 | 82.0% | 39005201 | 95.1% |
| | hours | | | | |

### 3.4. Conclusions

Based on the results from this study, the dual-pump ambulatory infusion configuration enables continuous administration of the DART-A DP dosing solution at room temperature with acceptable DART-A recovery for 24, 48, and 72 hours. The particle counts for all samples collected during continuous infusion were all very low and met USP specifications. The BA content in Stabilizer 1 used for DART-A DP dosing solution preparation remained at effective levels when the worst-case dilution scenario was tested. DART-A loaded in the medication cassette remained active and stable during continuous infusion at room temperature and even at elevated temperature (37° C.) under shaking (100 rpm) for up to 72 hours. These studies demonstrate that, the diluted DART-A DP formulation is compatible with the infusion components and can be used with the dual pump ambulatory infusion configuration for continuous administration of the DART-A DP formulation at room temperature. DART-A may be administered by loading the diluted DART-A DP formulation into the medication cassette on Day 1 to cover the drug dose needed for the first 2 days, and re-loading on day 3 to cover the drug dose needed for the next 2 days of treatment. The saline, pre-filled into the medication cassettes, is loaded onto the ambulatory pump 2 daily during the treatment cycle.

### Example 4

### DART-A Single Pump Compatibility Study Utilizing Stabilizer 2 Solution

This Example summarizes a compatibility and microbial challenge study performed using Stabilizer 2 for preparation of the DART-A DP dosing solution. The stability of DART-A in a single pump configuration at 20 ng/mL and at 0.1 mg/mL doses bracketing potential low and high dose concentrations was evaluated up to 72 hours after dose preparation and 96 hours post infusion and was shown to be acceptable. Based on the results of the study, 40 mL of Stabilizer 2 solution will be diluted in a 250 mL normal saline containing IV bag (having a nominal volume of 270 mL) before adding the DART-A DP formulation, to prepare the final dosing solution for administration using the single pump configuration. After storage for up to 72 hours, the dosing solution can be continuously administered at room temperature over 96 hours from the saline bag to support four-day continuous administration.

The DART-A DP formulation may be administered by a continuous IV infusion over four days using an ambulatory or infusion pump in a dosing solution preparation using Stabilizer 2 in 0.9% Sodium Chloride, USP (normal saline). Stabilizer 2 is added to a normal saline IV bag prior to adding the DART-A DP formulation to prevent adsorption of the diabody to the IV bag and IV tubing. When Stabilizer 2 is diluted in normal saline during dose preparation, the dosing solution used for administration will contain 1.7 mg/mL BA, 0.55 mg/mL MP, and 0.032 mg/mL PS80. PS80 is included in the formulation to prevent adsorption of the DART-A to the surfaces of IV bags and lines, and both BA and MP are included to prevent microbial growth during dose preparation, storage and administration.

After dilution during dose preparation, final dosing solutions will contain DART-A concentrations ranging from 20 ng/mL to about 1250 ng/mL to enable two to four-day (48-96 hour) administration of doses from 30 ng/kg/day to about up to 500 ng/kg/day. The amount calculated for dilution of DART-A is based upon the subject's weight (minimum and maximum ranges were used for simulation studies) and the desired dosing (Table 35).

Studies were performed to support the stability and compatibility of the DART-A DP formulation diluted in normal saline bags containing Stabilizer 2 and administration of this DART-A DP dosing solution using ambulatory and infusion pump IV configurations. The purpose of the studies was to demonstrate that the DART-A DP formulation is compatible with normal saline bags containing Stabilizer 2 in the desired dose range and with the intended infusion components.

TABLE 35
Illustrative Delivery of DART-A in Ambulatory Pump Configuration

| | | | Dosing | Delivered |
|---|---|---|---|---|
| | Patient | Total | Solution | Volume |
| Dose | Weight | Dose | Concentration | (mL) Per Day for |
| (ng/kg/day) | (kg) | (ng/day) | (ng/mL) | Four Days |
| 30 | 40 | 1200 | 20.0 | 60 |
| 500 | 150 | 75000 | 1250.0 | 60 |

Dosing solution preparation will require a two-step process involving addition of Stabilizer 2 to a normal saline bag followed by addition of the DART-A DP formulation. Representative low and high final dosing solution concentrations are summarized in Table 36.

TABLE 36
Dose Preparation Scheme for Low and High Doses

| | | Nominal | | | |
|---|---|---|---|---|---|
| | | Volume (mL) in | Volume | Volume | Total |
| Dose | Dose | 250 mL Normal | Stabilizer | DART-A | Volume |
| (ng/kg/day) | | Saline IV Bag | (mL) | (mL) | (mL) |
| 30 | 20 | 270.0 | 40 | 0.06 ^{A} | 310.06 |
| 500 | 1,250 | 270.0 | 40 | 3.90 ^{A} | 313.90 |

^{A} Dose prepared using 0.1 mg/mL DART-A DP formulation

The objectives of the studies were as follows:
(A) To evaluate the stability and recovery of diluted DART-A when the dose is prepared using Stabilizer 2 and stored at room temperature for 72 hours;
(B) To test the levels of the Stabilizer 2 solution preservatives when the prepared dosing solution is stored at room temperature for 72 hours, followed by administration; and
(C) To evaluate the compatibility and recovery of DART-A diluted in Stabilizer 2 solution with the single ambulatory pump infusion components during continuous infusion at room temperature 48 hours (10 ng/mL and 1,250 ng/mL) and 96 hours (20 ng/mL and 0.1 mg/mL) after room temperature storage.

A bracketed approach was used to test the highest concentration (1,250 ng/mL) and the lowest concentration (20 ng/mL), which correspond to the representative therapeutic doses of 500 ng/kg/day and 30 ng/kg/day, respectively. In addition, dosing solution concentrations of 10 ng/mL and 0.1 mg/mL (100,000 ng/mL) were also prepared and evaluated. The lower concentration of 10 ng/mL was evaluated to demonstrate the compatibility of the dosing solution below the minimum target dose for a 40 kg patient. The 0.1 mg/mL dosing solution concentration is outside of the target administration dosing range but was used as a dose concentration to evaluate to meet the limit of quantitation (LOQ) threshold of the size exclusion chromatography (SE-HPLC) method to characterize percent HMW species and capillary isoelectric focusing (cIEF) method to characterize drug product charge variants.

### 4.1. Study Design

Dose preparation for the compatibility study followed the proposed single ambulatory pump dose preparation scheme described in FIG. 13. As part of the compatibility study, DART-A protein and preservative stability was monitored during room temperature storage for 72 hours. After storage, administration followed the proposed single ambulatory pump dose administration configuration shown in FIG. 14.

Only the CADD administration set with IV spike was tested. Continuous administration of 240 mL of dosing solution (5 mL/hr for 48 hours or 2.5 mL/hr for 96 hours) was performed using the bracketing strategy for protein concentration. The methods utilized for dose preparation and administration are described in the following section.

### 4.2. Dosing Solution Preparation

Dosing solutions were prepared by adding 40 mL of Stabilizer 2 to a 250 mL normal saline bag (having a nominal volume of 270 mL) followed by the addition of a calculated amount of DART-A DP formulation to achieve the target drug concentration described above. The dosing solution was stored in the normal saline bag for 72 hours at room temperature while minimizing exposure to light during storage.

B. Braun eXcel normal saline bags were used for this study. Standard practice dictates that normal saline bags contain greater than the 250 mL volume stated on the packaging. The nominal fill volume, based on manufacturer specifications, is 270 mL for each B. Braun eXcel bag. This volume was used to calculate the excipient concentrations of Stabilizer 2 during formulation development to ensure the final concentrations of preservatives are at or below ADI levels. Dosing solution preparation will require a two-step process involving dilution of Stabilizer 2 in a saline bag followed by addition of DART-A DP formulation.

Each dose was prepared by adding 40 mL of Stabilizer 2 to the IV bag. Stabilizer 2 vials were shaken well before addition to the IV bag. After the stabilizer solution addition, each saline bag was gently mixed by inverting the bag for five minutes. After mixing, the predetermined volume of DART-A DP formulation (see Table 34) was added to each bag using either a 1 mL syringe (low dose preparations) or 5 mL syringe (high dose preparations). Each bag was thoroughly mixed again by inverting the bag for five minutes. After mixing, all the air was removed from the saline bag using a new 60 mL syringe.

After dose preparation, 15 mL from each bag was collected for appearance, pH, osmolality, and subvisible particulates T=0 hour analysis. All other analysis methods were completed upon generation of all samples. Each bag was incubated at room temperature for 72 hours. After 72 hours of storage, 15 mL of solution was collected for analysis from each of the bags before being infused to test the compatibility of the dosing solution with the administration components.

### 4.3. Dosing Solution Administration

After storage, the prepared dosing solution at each concentration was connected to the ambulatory pump via an IV bag spike coupled with a 0.22 μm filtered, non-DEHP PVC IV administration extension set and infused continuously over the course of 48 hours at 10 ng/mL and 1,250 ng/mL and 96 hours at room temperature for the lowest starting dose for four-day administration which is 20 ng/mL and also at 0.1 mg/mL.

Compatibility during dose administration was evaluated using Smiths Medical CADD Legacy-1 ambulatory pumps which were programmed to deliver the DART-A dose at either 5 mL/hr for 48 hours or 2.5 mL/hr for 96 hours. As seen in FIG. 14 above, a Smiths Medical CADD administration set with IV bag spike was used to directly connect the saline bag containing the dosing solution to the ambulatory pump. Infused DART-A DP dosing solution was collected in a 250 mL glass bottle that was pre-coated with Stabilizer 2 diluted in saline to prevent non-specific protein binding during sample collection. Excess Stabilizer 2 diluted in saline was removed from the bottle and allowed to dry before sample collection. After half of the infusion was complete, a new, empty, pre-coated 250 mL glass bottle replaced the previous collection bottle. This ensured the final, infused dosing solution accurately represented the hold time of a 48-hour or 96-hour sample. Samples were taken at the time of dose preparation (T=0 hours) and from the final dispensed pool in the collection vessel (post infusion sample, infusion T=48 hours and infusion T=96 hours) and tested for visual appearance, subvisible particulates, pH, osmolality, preservative concentration by reverse phase chromatography (RP-HPLC), protein recovery by fluorescent size exclusion chromatography (FLR SE-HPLC) and ELISA assay, and potency by CD3 and CD123 binding. Samples at 0.1 mg/mL concentration was analyzed for potency using a reporter gene bioassay.

### 4.4. Analytical Testing

All dosing solutions are analyzed upon dose preparation (T=0 hours), after room temperature storage (T=72 hours), and after infusion (Infusion time, T=48 hours or T=96 hours). The analytical tests that were used to analyze compatibility are listed below in Table 37 (NT: attribute was not tested at specified dose).

TABLE 37
Compatibility Attributes Tested for Each Dosing Solution

| | | | | | 0.1 |
|---|---|---|---|---|---|
| | | | | | mg/mL |
| | | | | 1250 | Simulated |
| | | | 20 | ng/mL | Dose |
| | | 10 ng/mL | ng/mL | Low | Dose |
| Attribute | | Low | Dose | High | DP Dose |
| Tested | Method | Solution | Solution | Solution | Solution |
| pH | pH | X | X | X | NTA |
| Appearance | Appearance | X | X | X | NT |
| Osmolality | Osmolality | X | X | X | NT |
| Protein | ELISA | X | X | NT | X |
| Recovery | | | | | |
| Protein | FLR SE- | NT | NT | X | NT |
| Recovery | HPLC | | | | |
| Subvisible | Light | X | X | X | NT |
| Particulate | Obscuration | | | | |
| Count | Method | | | | |
| | (HIAC) | | | | |
| Benzyl Alcohol | Reverse | X | X | X | NT |
| Recovery | Phase | | | | |
| | (RP)HPLC | | | | |
| Methylparaben | Reverse | X | X | X | NT |
| Recovery | Phase | | | | |
| | (RP)HPLC | | | | |
| Protein | CD3 and | NT | NT | X | NT |
| Potency | CD 123 | | | | |
| | Binding | | | | |
| Protein | Reporter | NT | X | NT | X |
| Potency | Gene | | | | |
| | BioAssay | | | | |
| Charge | Capillary | | | | |
| Variants | Isoelectric | NT | NT | NT | X |
| | Focusing | | | | |
| | (cIEF) | | | | |
| Protein Purity | FLR SE- | NT | NT | NT | X |
| | HPLC | | | | |

### 4.5 Results

The compatibility and stability results of the low and high dose solutions after 72-hour room temperature storage in IV bag followed by infusion using a single pump configuration at room temperature are listed below in Table 38.

TABLE 38
Compatibility and In-use Stability of DART-Dilute in Normal
Saline IV Bag Containing Stabilizer 2 Solution

| Analytical methods | | | |
|---|---|---|---|
| | 10 ng/mL | | |
| | | | Post Infusion |
| | | Post Storage | (Infusion Time = 48 |
| | T = 0 | (T = 72 Hrs) | Hrs) |
| pH | 8.1 | 8.1 | 8.1 |
| Visual Appearance ^{A} | C, L | C, L | C, L |
| | FNP, FPP | FNP, FPP | FNP, FPP |
| Osmolality (mOsm/kg) | 275 | 275 | 274 |
| % Protein recovery B | 100 | 99.8 | 88.3 |
| Subvisible Particles by | | | |
| USP <788> ^{C} | | | |
| ≥2 μm/mL | 206 | 192 | 140 |
| ≥10 μm/mL | 7 | 10 | 2 |
| ≥25 μm/mL | 1 | 1 | 1 |
| Preservative recovery by RP- | | | |
| HPLC | | | |
| % Benzyl alcohol D | 100 | 100.4 | 98.2 |
| % methyl 4-hydroxybenzoate | 100 | 101.0 | 98.2 |
| sodium ^{D} | | | |
| % Potency, CD3 binding ^{E} | NT ^{F} | NT ^{F} | NT ^{F} |
| % Potency, CD123 binding ^{E} | NT ^{F} | NT ^{F} | NT ^{F} |
| | 20 ng/mL | | |
| | | | Post Infusion |
| | | Post Storage | (Infusion Time = 96 |
| | T = 0 | (T =72 Hrs) | Hrs) |
| pH | 8.1 | 8.1 | 7.9 |
| | C, L | C, L | C, L |
| Visual Appeamnce ^{A} | FNP, FPP | FNP, FPP | FNP, FPP |
| Osmolality (mOsm/kg) | 276 | 276 | 276 |
| % Protein recovery ^{B} | 100 | 101.6 | 93.7 |
| Subvisible Particles by | | | |
| USP <788> ^{C} | | | |
| ≥2 μm/mL | 344 | 412 | 85 |
| ≥10 μm/mL | 8 | 8 | 9 |
| ≥25 μm/mL | 0 | 0 | 3 |
| Preservative recovery by RP- | | | |
| HPLC | | | |
| % Benzyl alcohol ^{D} | 100 | 99.7 | 97.0 |
| % methyl 4-hydroxybenzoate | 100 | 99.2 | 95.1 |
| sodium ^{D} | | | |
| % Potency, CD3 binding ^{E} | NT ^{F} | NT ^{F} | NT ^{F} |
| % Potency, CD123 binding ^{E} | NT ^{F} | NT ^{F} | NT ^{F} |
| | 1250 ng/mL | | |
| | | | Post Infusion |
| | | Post Storage | (Infusion Time = 48 |
| | T = 0 | (T = 72 Hrs) | Hrs) |
| pH | 8.0 | 8.0 | 8.0 |
| Appearance ^{A} | C, L | C, L | C, L |
| | FNP, FPP | FNP, FPP | FNP, FPP |
| Osmolality (mOsm/kg) | 275 | 275 | 276 |
| % Protein recovery ^{B} | 100 | 95.5 | 92.4 |
| Subvisible Particles by | | | |
| USP <788> ^{C} | | | |
| ≥2 μm/mL | 664 | 146 | 188 |
| ≥10 μm/mL | 21 | 7 | 4 |
| ≥25 μm/mL | 1 | 1 | 1 |
| Preservative recovery by RP- | | | |
| HPLC | | | |
| % Benzyl alcohol ^{D} | 100 | 99.9 | 99.3 |
| % methyl 4-hydroxybenzoate | 100 | 100.9 | 98.4 |
| sodium ^{D} | | | |
| % Potency, CD3 binding ^{E} | 111 | 101 | 92 |
| % Potency, CD123 binding ^{E} | 146 | 140 | 123 |
| free from | | | |

^{A} Visual appearance: Clear (C) and Colorless (L); Particulates: FNP = Essentially free from visible foreign particles, FPP = Essentially proteinaceous particles.
^{B} DART-A recovery of the post-infusion sample is expressed as a percentage relative to the corresponding T = 0 sample. Protein recovery was calculated using FLR SE-HPLC peak areafor the 1,250 ng/mL dose or by ELISA quantitation (CD3 binding) forthe other doses.
^{C} Sub-visible particulates weremeasured by USP <788> light obscuration (Beckman CoulterHIAC).
^{D} Preservative recovery is expressed as the % Monomer, measured by RP-HPLC.
^{E} Potency is reported as Relative Potency (%), calculated relative to a DART-A reference standard.
^{F} NT: Not tested as dose concentrationis below LOQ for the qualified analytical method.

All solutions were observed to be clear, colorless with no visible particulates upon dose preparation (T=0 hours), after storage (T=72 hours), and post infusion (infusion T=48 hours or infusion T=96 hours). Protein recovery was calculated using SE-HPLC method for the 1,250 ng/mL dosing solution or by ELISA (CD3 binding) for all other solutions. The % protein recovery (protein concentration calculated relative to T=0) of DART-A post-infusion was acceptable for dosing solutions tested. In addition, no significant changes in pH, visual appearance, and osmolality in post-infusion samples were observed when compared to T=0. Subvisible particle counts for particles ≥2 μm, ≥10 μm and ≥25 μm did not increase in post-infusion samples for dosing solutions compared to T=0. Overall, the results in Table 37 show no significant product changes in pH, protein recovery, potency, sub-visible particles and visual appearance when dosing solutions are administered with an ambulatory infusion pump. Longer infusion time of 96 hours at lowest potential dose concentration of 20 ng/mL for four-day administration had no impact on product quality.

The DART-A characterization results for the 0.1 mg/mL final solution are shown below in Table 39.

TABLE 39
DART-A (0.1 mg/mL) Characterization Results After 72-Hour
Room Temperature Storage and Administration

| | | Time Point | | |
|---|---|---|---|---|
| | | | | Post Infusion |
| Test | | T = 0 | Post Storage | (Infusion Time = |
| Performed | Attribute Tested | Hrs | (Time = 72 Hrs) | 96 Hrs) |
| % Protein | Concentration (mg/mL) | 0.098 | 0.096 | 0.092 |
| Recovery ^{A} | % T₀ | 100 | 97.8 | 94.4 |
| Reporter Gene | Relative Potency (%) ^{B} | 136 | 88 | 80 |
| BioAssay | | | | |
| cIEF ^{C} | % Main Peak | 67.3 | 67.0 | 65.2 |
| | % AV | 28.3 | 29.1 | 31.4 |
| | % BV | 4.4 | 3.9 | 3.4 |
| | % Main Peak | 98.4 | 98.4 | 98.2 |
| FLR SE-HPLC ^{D} | % HMW | 0.8 | 0.8 | 0.9 |
| | % LMW | 0.9 | 0.9 | 0.9 |

^{A} Protein recovery was calculated by ELISA quantitation (CD3 binding)
^{B} Potency is reported as Relative Potency (%), calculated relative to the DART-A ReferenceStandard
^{C} AV = acidic variants, BV = basic variants
^{D} HMW = high molecular weight species, LMW =low molecular weight species

Table 39 shows no changes in the LMW and LMW by SE-HPLC at this concentration. Charge heterogeneity, as monitored by cIEF, shows minor changes within the variability of the analytical method. The relative potency values measured using the reporter gene bioassay are within the acceptance criteria (50-150%) for the method. No change in visual appearance was observed during storage and administration. Microbial growth data for the dose formulated solutions evaluated using the USP recommended microbes supports the use of the dosing solution for four-day continuous administration.

### 4.6. Conclusions

The compatibility study demonstrates that the DART-A DP formulation is stable when diluted in Stabilizer 2-stabilized normal saline solution bags at the lowest (20 ng/mL) and highest (1,250 ng/mL) dose concentrations tested and stored for 72 hours at room temperature. The compatibility study also shows that the dosing solution is compatible when administered by infusion over the course of 96 hours at room temperature using a single pump. Microbial growth data supports the use of the dosing solution for four-day continuous administration.

### Example 5

### Four and Seven Day Administration of DART-A Utilizing Stabilizer 2 Solution

This Example summarizes one representative scheme for a compatibility and microbial challenge study using Stabilizer 2 for preparation of the DART-A DP dosing solution for 4-day and 7-day administration of DART-A. The stability of DART-A in an IV bag or cassette with at-home single ambulatory pump at 30-500 ng/kg/day doses will be evaluated for 4-day and 7-day administration using a low flow rate of about 0.3 mL/hour to about 0.5 mL/hour. The stability of DART-A in a single pump configuration at 3250 ng/mL (3.25 mg/mL) doses will be evaluated up to 24 hours after dose preparation and 4 days (96 hours) and 7 days (168 hours) on storage. Based on the results of the study, Stabilizer 2 solution will be diluted in a normal saline containing IV bag before adding the DART-A DP formulation, to prepare the final dosing solution for administration using the single pump configuration. After storage for up to 24 hours, the dosing solution can be continuously administered at room temperature over 4 days (96 hours) or 7 days (168 hours) from the saline bag to support 4-day or 7-day continuous administration.

### Four-Day Administration

The DART-A DP formulation may be administered by a continuous IV infusion over 4 days using an ambulatory pump equipped with an IV bag or cassette. The amount calculated for dilution of the DART-A DP formulation is based upon the subject's weight (an average body weight of 78 kg was used for simulation studies). Table 40 describes an exemplary scheme of the steps for preparing the dosing solution. The DART-A DP formulation is diluted in a dosing solution using Stabilizer 2 in 0.9% Sodium Chloride, USP (normal saline). Normal saline (45 mL) is added to an empty IV bag. Stabilizer 2 (8 mL) is then added to the IV bag prior to adding the DART-A DP formulation (1.95 mL) to prevent adsorption of the diabody to the IV bag and IV tubing. When Stabilizer 2 is diluted in normal saline during dose preparation, the dosing solution (60 mL total volume) used for administration contains about 1.8 mg/mL BA, about 0.56 mg/mL MP, and about 0.033 mg/mL PS80. After dilution during dose preparation, the final dosing solution will contain a DART-A concentration of about 3250 ng/mL ng/mL to enable 4-day administration of a 500 ng/kg/day dose.

TABLE 40
Exemplary Four-Day Administration of DART-A
With Stabilizer 2 in a 60 mL Volume Usin
g an IV Bag or Cassette and an Ambulatory Pump

| | |
|---|---|
| Patient Weight | 78 kg |
| DART-A Dose | 500 ng/kg/day |
| Administration Flow | 0.5 mL/hour |
| Rate | |
| Total Dose Volume | 48 mL |
| Delivered Over 4 Days | |
| Total Minimum IV | |
| Bag Volume Including | 60 mL |
| Prime Volume | |
| Dose Preparation | 1. Add 45 mL of normal saline (0.9% |
| Steps | sodium chloride) to an empty IV bag. |
| | 2. Add 8 mL of Stabilizer 2 to the IV bag |
| | containing normal saline. |
| | 3. Add 1.95 mL DART-A DP formulation |
| | (0.1 mg/mL) to the IV bag. |
| | 4. Add 5.05 mL of normal saline to bring |
| | the final volume of the dosing |
| | solution in the IV bag to 60 mL |
| | DART A: 3250 ng/mL |
| | (3.25 mg/mL; 0.325%) |
| Final Concentrations | Benzyl Alcohol: 1.8 mg/mL (0.18%) |
| in Solution | Methylparaben: 0.56 mg/mL (0.056%) |
| | Polysorbate 80: 0.033 mg/mL (0.0033%) |

### Seven Day Administration

The DART-A DP formulation may also be administered by a continuous IV infusion over 7 days using an ambulatory pump equipped with an IV bag or cassette. The amount calculated for dilution of DART-A is based upon the subject's weight (an average body weight of 78 kg was used for simulation studies). Table 41 describes an exemplary scheme of the steps for preparing the dosing solution. The DART-A DP formulation is diluted in a dosing solution using Stabilizer 2 in 0.9% Sodium Chloride, USP (normal saline). Normal saline (70 mL) is added to an empty IV bag. Stabilizer 2 (18 mL) is then added to the IV bag prior to adding the DART-A DP formulation (3.25 mL) to prevent adsorption of the diabody to the IV bag and IV tubing. When Stabilizer 2 is diluted in normal saline during dose preparation, the dosing solution (100 mL total volume) used for administration will contain about 2.4 mg/mL BA, about 0.77 mg/mL MP, and about 0.047 mg/mL PS80. After dilution during dose preparation, the final dosing solution will contain a DART-A concentration of about 3250 ng/mL to enable 7-day administration of a 500 ng/kg/day dose.

TABLE 41
Exemplary Seven-Day Administration of
DART-A With Stabilizer 2 in a 100 mL
Volume Using an IV Bag or Cassette and an Ambulatory Pump

| | |
|---|---|
| Patient Weight | 78 kg |
| DART-A Dose | 500 ng/kg/day |
| Flow rate | 0.5 mL/hour |
| Total Dose Volume | 84 mL |
| Delivered Over 4 Days | |
| Total Minimum IV | 100 mL |
| Bag Volume Including | |
| Prime Volume | |
| Dose Preparation | 1. Add 70 mL of normal saline (0.9% |
| Steps | sodium chloride) to an empty IV bag. |
| | 2. Add 18 mL of Stabilizer 2 to the IV |
| | bag containing normal saline. |
| | 3. Add 3.25 mL DART-A DP |
| | formulation to the IV bag. |
| | 4. Add 8.75 mL of normal saline to bring |
| | the final volume of the dosing |
| | solution in the IV bag to 100 ml. |
| Final Concentrations | DART A: 3250 ng/mL (3.25 mg/mL; 0.325%) |
| in Solution | Benzyl Alcohol: 2.4 mg/mL (0.24%) |
| | Methylparaben: 0.77 mg/mL (0.077%) |
| | Polysorbate 80: 0.047 mg/mL (0.0047%) |

Studies are ongoing to support the stability and compatibility of the DART-A DP formulation diluted in normal saline bags containing Stabilizer 2 and 4-day and 7-day administration of this DART-A DP dosing solution using ambulatory IV bag and cassette configurations as shown in Table 40 and Table 41. The purpose of the studies is to demonstrate that the DART-A DP formulation is compatible with normal IV bags containing Stabilizer 2 at the desired 30-500 ng/kg/day dose and with the intended infusion components for 4-day and 7-day administration at a flow rate of about 0.3 mL/hour to about 0.5 mL/hour. The objectives of the studies are to evaluate the compatibility and recovery of DART-A diluted in Stabilizer 2 solution with the single ambulatory pump infusion components during continuous infusion at room temperature for 4 days (96 hours) and 7 days (168 hours) after room temperature storage.

### 5.1. Study Design

Dose preparation for the compatibility study may follow the proposed single ambulatory pump dose preparation scheme as described in FIG. 13. As part of the compatibility study, DART-A protein and preservative stability will be monitored during room temperature storage for 24 hours. After storage, administration may follow the proposed single ambulatory pump dose administration configuration shown in FIG. 14.

Continuous administration of 48 mL of dosing solution (0.5 mL/hr for 4 days (96 hours)) for 4-day administration or continuous administration of 84 mL of dosing solution (0.5 mL/hr for 7 days (168 hours)) will be performed. The methods which will be utilized for dose preparation and administration are described in the following section.

### 5.2. Dosing Solution Preparation

Dosing solutions will be prepared by as shown in Table 40 (4-day administration) and Table 41 (7-day administration) to achieve the target drug concentration suitable for a 78 kg patient at the 500 ng/kg/day dose. The dosing solution will be stored in the normal IV bag for 24 hours at room temperature while minimizing exposure to light during storage.

Dosing solution preparation will require a three-step process involving addition of normal saline to an empty intravenous (IV) bag, dilution of Stabilizer 2 in the normal saline containing IV bag followed by addition of DART-A DP formulation. Stabilizer 2 vials will be shaken well before addition to the IV bag. After the stabilizer solution addition, each IV bag will be gently mixed by inverting the bag for five minutes. After mixing, the predetermined volume of DART-A DP formulation (see Table 40 or Table 41) will be added to each bag. Each bag will be thoroughly mixed again by inverting the bag for five minutes. After mixing, all the air will be removed from the IV bag using a syringe.

After dose preparation, an aliquot from each bag will be collected for appearance, pH, osmolality, and subvisible particulates T=0 hour analysis. All other analysis methods will be completed upon generation of all samples. Each bag will be incubated at room temperature for 24 hours to 8 days. After the completion of the storage time, an aliquot of dosing solution will be collected from each of the bags for analysis before being infused to test the compatibility of the dosing solution with the administration components.

### 5.3. Dosing Solution Administration and Analytical Testing

An example of how the prepared dosing solution can be administered is described. After storage, the prepared dosing solution will be connected to the ambulatory pump via an IV bag spike coupled with a filtered, administration extension set and infused continuously over the course of 4 days (96 hours) or 7 days (168 hours) at room temperature. Compatibility during dose administration will be evaluated using ambulatory pumps (e.g., Smiths Medical CADD Legacy-1 ambulatory pumps) which will be programmed to deliver the DART-A dose at either 0.3 mL/hour or 0.5 mL/hour for 4 days (96 hours) or 0.3 mL/hour or 0.5 mL/hour for 7 days (168 hours). As shown in FIG. 14 above, a Smiths Medical CADD administration set with IV bag spike may be used to directly connect the saline bag containing the dosing solution to the ambulatory pump. Infused DART-A DP dosing solution will be collected in a glass bottle that is rinsed with Stabilizer 2 diluted in saline to remove foreign particles during sample collection. Excess Stabilizer 2 diluted in saline will be removed from the bottle and allowed to dry before sample collection. After half of the infusion is complete, a new, empty, glass bottle will replace the previous collection bottle. This will ensure that the final, infused dosing solution accurately represented the hold time of a 4-day (96 hour) or 7-day (168 hour) sample. Samples will be taken at the time of dose preparation (T=0 hours) and from the final dispensed pool in the collection vessel (post-infusion sample, infusion T=4 days/96 hours and infusion T=7 days/168 hours) and analytical testing will be performed essentially as described herein (see in particular, Example 7) for visual appearance, subvisible particulates, pH, osmolality, preservative concentration by reverse phase chromatography (RP-HPLC), protein recovery by fluorescent size exclusion chromatography (FLR SE-HPLC) and ELISA assay, and potency by reporter gene bioassay.

### Example 6

### Four and Seven Day Administration of a Bispecific Diabody Utilizing Stabilizer 3 Solution

This Example summarizes one representative scheme for a compatibility and microbial challenge study using Stabilizer 3 (comprising 150 mM sodium chloride and 0.1 mg/mL PS80, pH 6.0) for preparation of the a bispecific diabody dosing solution, such as a DART-A DP dosing solution, for 4-day and 7-day administration of the bispecific diabody (e.g., DART-A). The stability of the bispecific diabody in an IV bag or cassette with at-home ambulatory pump at 30-500 ng/kg/day doses will be evaluated for 4-day and 7-day administration using a low flow rate of about 0.3 mL/hour to about 0.5 mL/hour. The stability of the bispecific diabody in a single pump configuration at 25 ng/mL to 3250 ng/mL (3.25 mg/mL) doses will be evaluated up to 24 hours after dose preparation and 4 days (96 hours) and 7 days (168 hours) on storage. Based on the results of the study, Stabilizer 3 solution will be diluted in a bacteriostatic saline containing IV bag before adding the bispecific diabody formulation, to prepare the final dosing solution for administration using the single pump configuration. After storage for up to 24 hours, the dosing solution can be continuously administered at room temperature over 4 days (96 hours) or 7 days (168 hours) from the saline bag to support 4-day or 7-day continuous administration.

Stability Studies

Stability studies were performed to evaluate the compatibility and overall recovery of a representative three chain bispecific diabody (“BD”). The BD comprises an Fc domain and is capable of binding CD3 and B7-H3 during continuous infusion at room temperature (22° C.±2° C.) for 6 hours. The bispecific diabody formulation was diluted with Stabilizer 3 to prepare the bispecific diabody dosing solution. The study tested the stability of the bispecific diabody in a disposable syringe. Samples were collected at T=0 and at 6 hours. Stability of the bispecific diabody was measured using SE-HPLC. Sample collected were also subjected to particle count testing. As shown in Table 42 and Table 43, the particle counts for all samples were low and met USP specifications.

TABLE 42
Six-Hour Stability of a Bispecific Diabody (BD) Diluted in Stabilizer 3
and Administered at 8 mL/Hour with a Single Pump

| | | | BD Recovery | Particle | Particle |
|---|---|---|---|---|---|
| Dose | | | (Relative | Counts/mL | Counts/mL |
| Concentration | Dose Samples | BD Concentration | T = 0) | ≥10 μm | ≥25 μm |
| 10,000 μg/mL | Dose | 9.90 mg/mL | NA | 37 | 6 |
| | Preparation | | | | |
| | (T =0) | | | | |
| | Post infusion | 9.93 mg/mL | 100.3% | 17 | 2 |
| 1 μg/mL | Dose | | | | |
| | Preparation | 0.89 μg/mL | NA | 3 | 0 |
| | (T = 0) | | | | |
| | Post infusion | 0.99 μg/mL | 111.2% | 3 | 2 |

TABLE 43
Six-Hour Stability of a Bispecific Diabody (BD) Diluted in Stabilizer 3
and Administered at 30 mL/Hour with a Single Pump

| Dose | Dose | BD Recovery | | |
|---|---|---|---|---|
| (Relative Theoretical | Counts/mL | Counts/mL | Particle | Particle |
| Concentration | Samples | Concentration) | ≥10 μm | ≥25 μm |
| 1,000 μg/mL | Dose | 88% | 13 | 5 |
| | Preparation | | | |
| | (T =0) | | 7 | 0 |
| | Post infusion | 101% | | |
| 1 μg/mL | Dose | | 19 | 1 |
| | Preparation | 111% | 0 | |
| | (T =0) | | 1 | 1 |
| | Post infusion | 106% | | |

These short-term (6-hour) stability studies demonstrate that a low-dose, diluted bispecific diabody formulation is compatible with both Stabilizer 3 and the infusion components. The diluted bispecific diabody formulation and Stabilizer 3 can be used with the single pump configuration for continuous administration of the bispecific diabody formulation at room temperature with acceptable bispecific diabody recovery and low particle counts.

### Four-Day Administration

The bispecific diabody formulation (e.g., DART-A DP formulation) may be administered by a continuous IV infusion over 4 days using a single ambulatory pump equipped with an IV bag or cassette. The amount calculated for dilution of the bispecific diabody formulation is based upon the subject's weight (an average body weight of 78 kg was used for simulation studies). Table 44 describes an exemplary scheme of the steps for preparing a DART-A DP dosing solution. The DART-A DP formulation is diluted in a dosing solution using Stabilizer 3 in bacteriostatic saline (0.9% BA, 0.9% Sodium Chloride, USP). Bacteriostatic saline (45 mL) is added to an empty IV bag. Stabilizer 3 (8 mL) is then added to the IV bag prior to adding the DART-A DP formulation (1.95 mL) to prevent adsorption of DART-A to the IV bag and IV tubing. When Stabilizer 3 is diluted in bacteriostatic saline during dose preparation, the dosing solution (60 mL total volume) used for administration contains about 7.5 mg/mL BA and about 0.013 mg/mL PS80. After dilution during dose preparation, the final dosing solution will contain a DART-A concentration of about 3250 ng/mL ng/mL to enable 4-day administration of a 500 ng/kg/day dose.

TABLE 44
Exemplary Four-Day Administration of DART-A
With Stabilizer 3 in a 60 mL Volume Using an IV
Bag or Cassette and an Ambulatory Pump

| | |
|---|---|
| Patient Weight | 78 kg |
| DART-A Dose | 500 ng/kg/day |
| Administration Flow | 0.5 mL/hour |
| Rate | |
| Total Dose Volume | 48 mL |
| Delivered Over 4 Days | |
| Total Minimum IV | 60 mL |
| Bag Volume Including | |
| Prime Volume | |
| Dose Preparation | 1. Add 45 mL of bacteriostatic saline (0.9% |
| Steps | BA in normal saline) to an empty IV bag. |
| | 2. Add 8 mL of Stabilizer 3 to the IV bag |
| | containing bacteriostatic saline. |
| | 3. Add 1.95 mL DART-A DP formulation |
| | (0.1 mg/mL) to the IV bag. |
| | 4. Add 5.05 mL of bacteriostatic |
| | saline to bring the final |
| | volume of the dosing solution |
| | in the IV bag to 60 mL |
| Final Concentrations | DART A: 3250 ng/mL (3.25 mg/mL; 0.325%) |
| in Solution | Benzyl Alcohol: 7.5 mg/mL (0.75%) |
| | Polysorbate 80: 0.013 mg/mL (0.0013%) |

### Seven-Day Administration

The bispecific diabody formulation (e.g., DART-A DP formulation) may also be administered by a continuous IV infusion over 7 days using a single ambulatory pump equipped with an IV bag or cassette. The amount calculated for dilution of the bispecific diabody is based upon the subject's weight (an average body weight of 78 kg was used for simulation studies). Table 45 describes an exemplary scheme of the steps for preparing a DART-A DP dosing solution. The DART-A DP formulation is diluted in a dosing solution using Stabilizer 3 in bacteriostatic saline (0.9% BA, 0.9% Sodium Chloride, USP). Bacteriostatic saline (70 mL) is added to an empty IV bag. Stabilizer 3 (18 mL) is then added to the IV bag prior to adding the DART-A DP formulation (3.25 mL) to prevent adsorption of DART-A to the IV bag and IV tubing. When Stabilizer 3 is diluted in bacteriostatic saline during dose preparation, the dosing solution (100 mL total volume) used for administration will contain about 7.5 mg/mL BA and about 0.013 mg/mL PS80. After dilution during dose preparation, the final dosing solution will contain a DART-A concentration of about 3250 ng/mL to enable 7-day administration of a 500 ng/kg/day dose.

TABLE 45
Exemplary Seven-Day Administration of DART-A
With Stabilizer 3 in a 100 mL Volume
Using an IV Bag or Cassette and an Ambulatory Pump

| | |
|---|---|
| Patient Weight | 78 kg |
| DART-A Dose | 500 ng/kg/day |
| Flow rate | 0.5 mL/hour |
| Total Dose Volume | 84 mL |
| Delivered Over 4 Days | |
| Total Minimum IV | 100 mL |
| Bag Volume Including | |
| Prime Volume | |
| Dose Preparation | 1. Add 70 mL of bacteriostatic saline (0.9% |
| Steps | BA in normal saline) to an empty IV bag. |
| | 2. Add 18 mL of Stabilizer 3 to the IV bag |
| | containing bacteriostatic saline. |
| | 3. Add 3.25 mL DART-A DP formulation |
| | to the IV bag. |
| | 4. Add 8.75 mL of bacteriostatic saline |
| | to bring the final volume of the dosing |
| | solution in the IV bag to 100 ml. |
| Final Concentrations | DART A: 3250 ng/mL (3.25 mg/mL; 0.325%) |
| in Solution | Benzyl Alcohol: 7.5 mg/mL (0.75%) |
| | Polysorbate 80: 0.013 mg/mL (0.0013%) |

Studies are ongoing to support the stability and compatibility of the DART-A DP formulation diluted in bacteriostatic saline bags containing Stabilizer 3 and 4-day and 7-day administration of this DART-A DP dosing solution using ambulatory IV bag and cassette configurations as shown in Table 44 and Table 45. The purpose of the studies is to demonstrate that the DART-A DP formulation is compatible with bacteriostatic saline bags containing Stabilizer 3 at the desired 30-500 ng/kg/day dose and with the intended infusion components for 4-day and 7-day administration at a flow rate of about 0.3 mL/hour to about 0.5 mL/hour. The objectives of the studies are to evaluate the compatibility and recovery of DART-A diluted in Stabilizer 3 solution with the single ambulatory pump infusion components during continuous infusion at room temperature for 4 days (96 hours) and 7 days (168 hours) after room temperature storage.

### 6.1. Study Design

Dose preparation for the compatibility study may follow the proposed single ambulatory pump dose preparation scheme as described in FIG. 13. As part of the compatibility study, the DART-A protein and preservative stability will be monitored during room temperature storage for 24 hours. After storage, administration may follow the proposed single ambulatory pump dose administration configuration shown in FIG. 14.

Continuous administration of 48 mL of dosing solution (0.5 mL/hr for 4 days (96 hours)) for 4-day administration or continuous administration of 84 mL of dosing solution (0.5 mL/hr for 7 days (168 hours)) will be performed. The methods which will be utilized for dose preparation and administration are described in the following section.

### 6.2. Dosing Solution Preparation

Dosing solutions will be prepared by as shown in Table 44 (4-day administration) and Table 45 (7-day administration) to achieve the target drug concentration suitable for a 78 kg patient at the 500 ng/kg/day dose. The dosing solution will be stored in the normal IV bag for 24 hours at room temperature while minimizing exposure to light during storage.

Dosing solution preparation will require a three-step process involving addition of normal saline to an empty intravenous (IV) bag, dilution of Stabilizer 3 in the normal saline containing IV bag followed by addition of DART-A DP formulation. Stabilizer 3 vials will be shaken well before addition to the IV bag. After the stabilizer solution addition, each IV bag will be gently mixed by inverting the bag for five minutes. After mixing, the predetermined volume of the DART-A DP formulation (see Table 44 or Table 45) will be added to each bag. Each bag will be thoroughly mixed again by inverting the bag for five minutes. After mixing, all the air will be removed from the IV bag using a syringe.

After dose preparation, an aliquot from each bag will be collected for appearance, pH, osmolality, and subvisible particulates T=0 hour analysis. All other analysis methods will be completed upon generation of all samples. Each bag will be incubated at room temperature for 24 hours to 8 days. After the completion of the storage time, an aliquot of dosing solution will be collected from each of the bags for analysis before being infused to test the compatibility of the dosing solution with the administration components.

### 6.3. Dosing Solution Administration and Analytical Testing

An example of how the prepared dosing solution can be administered is described. After storage, the prepared dosing solution will be connected to the ambulatory pump via an IV bag spike coupled with a filtered, administration extension set and infused continuously over the course of 4 days (96 hours) or 7 days (168 hours) at room temperature. Compatibility during dose administration will be evaluated using ambulatory pumps (e.g., Smiths Medical CADD Legacy-1 ambulatory pumps) which will be programmed to deliver the DART-A dose at either 0.3 mL/hour or 0.5 mL/hour for 4 days (96 hours) or 0.3 mL/hour or 0.5 mL/hour for 7 days (168 hours). As shown in FIG. 14 above, a Smiths Medical CADD administration set with IV bag spike may be used to directly connect the bacteriostatic saline bag containing the dosing solution to the ambulatory pump. Infused DART-A DP dosing solution will be collected in a glass bottle that is rinsed with Stabilizer 3 diluted in bacteriostatic saline to remove foreign particles during sample collection. Excess Stabilizer 3 diluted in bacteriostatic saline will be removed from the bottle and allowed to dry before sample collection. After half of the infusion is complete, a new, empty, glass bottle will replace the previous collection bottle. This will ensure that the final, infused dosing solution accurately represented the hold time of a 4-day (96 hour) or 7-day (168 hour) sample. Samples will be taken at the time of dose preparation (T=0 hours) and from the final dispensed pool in the collection vessel (post-infusion sample, infusion T=4 days/96 hours and infusion T=7 days/168 hours) and analytical testing will be performed essentially as described herein (see in particular, Example 6) for visual appearance, subvisible particulates, pH, osmolality, preservative concentration by reverse phase chromatography (RP-HPLC), protein recovery by fluorescent size exclusion chromatography (FLR SE-HPLC) and ELISA assay, and potency by reporter gene bioassay.

### Example 7

### Materials and Methods

### 7.1. HIAC Liquid Particle Counting

Particle count analysis was carried out according to USP <787>. Analysis was performed only on samples stored at 5±3° C. In summary, each sample was warmed to room temperature before a 60-minute degassing. Thermo Scientific Standards and RODI water were analyzed to ensure proper machine function. To minimize carryover, samples were analyzed from low to high concentration. In between samples, the probe was rinsed with water.

7.2. Differential Scanning calorimetry (DSC)

Differential Scanning calorimetry measures the energetics of protein unfolding during a temperature ramp. Peaks represent apparent melting temperatures and the area under the peaks represent the enthalpy of the thermal transition. Data were collected for duplicate samples over 20° C. to 90° C. range using temperature ramp rate of 60° C./hour.

7.3. Capillary Isoelectric Focusing (cIEF)

cIEF analysis was performed to characterize drug product charge variants. The configuration included a iCE System with Alcott 720NV Autosampler. Samples were prepared to a final concentration of 0.1 mg/mL and centrifuged at 5±3° C. for 40 minutes prior to analysis.

### 7.4. Liquid Chromatography Electrospray Ionization Mass Spectrometry (LC-ESIMS)

Intact molecule was analyzed with electrospray ionization mass spectrometry (ESI-MS) following a LC separation, for RS and aged DP samples. Prior to analysis, PS80 in aged DP was removed with a detergent-OUT Tween spin column. Samples were then analyzed by LC-ESI-MS. The mass spectrum of multiply charged ions of the protein generated by electrospray ionization (ESI) were deconvoluted using MaxEnt1 algorithm to provide the molecular weight of the intact molecule.

7.5. Size Exclusion High Performance Liquid Chromatography with Fluorescence Detection (FLR SE-HPLC)

FLR SE-HPLC analysis was performed to measure protein purity, monomer purity and protein stability. The FLR SE-HPLC configuration included a fluorescence (FLR) detector (λ_{X}=280 nm, λ_{E}=340 nm), a mobile phase composition of 350 mM Mobile Phase (175 mMNaH2PO4, 175 mMNa2SO, pH 6.3) and a TosoBio TSK G3000SW_{XL} size exclusion column. Samples were diluted to 0.1 mg/mL and stored at 5±3° C. for 24 to 96 hours prior to analysis. Eight μg of protein (or 100 μL is sample concentration was below 0.1 mg/mL) was injected for each 30-minute run. HMW and LMW species and monomer peaks were monitored.

### 7.6. Tryptic Peptide Mapping

Reduced peptide mapping experiments were carried out using LC-MS on Waters Acuity UPLC H-Class Bio coupled to Waters Xevo G2-XS QTof instrument. All experiments were acquired using MSE acquisition to confirm peptide sequence and to elucidate amino acid modifications. The samples were processed using the Waters UNIFI Biopharmaceutical System Solution.

7.7. ELISA (rhIL3Rα)

In brief, the assay plate was coated with soluble recombinant human IL-3 receptor alpha (rhIL3Rα) overnight. After blocking the non-specific sites with 5% bovine serum albumin (BSA) in phosphate buffered saline (PBS), the plate was incubated with DART-A standard calibrators, quality controls and test samples. The DART-A present in the standard calibrators, quality controls and test samples were captured by the immobilized rhIL3Rα. The captured DART-A was detected by the addition of 2A5-Biotin (biotinylated antibody recognizing the E-coil/K-coil anti-(EK) antibody heterodimerization region of DART proteins) and Sulfo-TAG labeled Streptavidin (an electrochemiluminescence (ECL) label). After addition of MSD Read Buffer, the plate was inserted into the Sector Imager 2400 Plate Reader. When a voltage was applied to the plate electrodes, the bound Sulfo-TAG label produces an ECL signal, which was captured by the plate reader. A quantitative measure of the ECL signal emitted by each well was recorded, and the ECL counts emitted by the standard calibrators were used to generate a standard curve using a 4-parameter logistic (4PL) fit. The concentrations of DART-A samples were then interpolated from the samples' ECL counts and the equation describing the standard curve.

### 7.8. CD3 Binding Assays

The indirect enzyme-linked immunosorbent assay (ELISA) was used to evaluate the potency of the anti-CD3 arm of DART-A, by quantifying binding of DART-A to an immobilized, soluble recombinant human CD3 (shCD3-Fos-Jun) in an ELISA. The two CD3 chains (delta and gamma) contain either the Fos protein or the Jun protein to enhance proper folding and functionality of the CD3 complex. Specificity of the ELISA for DART-A is provided by recognition of the CD3 antigen by DART-A, and subsequent detection of bound DART-A using a specific antibody for the E/K coil. The soluble CD3-Fos-Jun, was coated on the surface of an ELISA plate. DART-A sample was added and allowed to bind to the shCD3-Fos-Jun. Detection of bound DART-A was accomplished with a biotin conjugated antibody (1F5-Bt), which recognizes the E/K coil of the DART molecule. Quantification of bound probe antibody was achieved by addition of a colorimetric AP substrate (AP-Yellow 1 Component Substrate). Oxidation of AP-Yellow by AP yields a colored product that can be read at 405±10 nm. The assay was quantified by the intensity of color, measured using a spectrophotometer set to an optimal wavelength of 405±10 nm. Data were fitted to a four-parameter model with the following variable constraints: the maximum, minimum and slope of each dose response curve is set to be shared between all dose response curves on the plate; the EC₅₀ was determined independently for each curve. Each assay plate contained a DART-A Calibration Reference Standard sample with one to two test articles and a second set of reference standard (positive control) to control for intra plate variability. An assay run may consist of multiple plates, provided that a Calibration and a positive control Reference Standard samples are included on each plate. The Reportable Result is the Relative Potency, calculated as EC₅₀ values of the DART-A Reference Standard calibration curve divided by the EC₅₀ of the Test Article.

### 7.9. CD123 Binding Assays

The indirect enzyme-linked immunosorbent assay (ELISA) was used to evaluate the potency of the anti-CD123 arm of DART-A, by quantifying binding of DART-A to an immobilized, recombinant Interleukin 3 Receptor (rhIL3-R) in an ELISA. Specificity of the ELISA for DART-A is provided by recognition of the IL3R antigen by DART-A, and subsequent detection of bound DART-A using a specific antibody for the E/K coil. The soluble IL3R was coated on the surface of an ELISA plate. DART-A sample was added and allowed to bind to the IL3-R. Detection of bound DART-A was accomplished with a biotin conjugated antibody (1F5-Bt), which recognizes the E/K coil of the DART molecule. Quantification of bound probe antibody was achieved by addition of a colorimetric AP substrate (AP-Yellow 1 Component Substrate). Oxidation of AP-Yellow by AP yields a colored product that can be read at 405±10 nm. The assay was quantified by the intensity of color, measured using a spectrophotometer set to an optimal wavelength of 405±10 nm. Data were fitted to a four-parameter model with the following variable constraints: the maximum, minimum and slope of each dose response curve is set to be shared between all dose response curves on the plate; the EC₅₀ was determined independently for each curve. Each assay plate contained a DART-A Calibration Reference Standard sample with one to two test articles and a second set of reference standard (positive control) to control for intra plate variability. An assay run may consist of multiple plates, provided that a Calibration and a positive control Reference Standard samples are included on each plate. The Reportable Result is the Relative Potency, calculated as EC₅₀ value of the DART-A Reference Standard calibration curve divided by the EC₅₀ of the Test Article.

### 7.10. Reporter Gene BioAssay

Antibody-based or antibody-like drugs can be used to target and kill cancer cells through Antibody dependent cell-mediated cytotoxicity (ADCC). This ADCC-like Reporter Bioassay uses Jurkat effector cell lines (Promega, Inc.) that have been engineered with a NFAT response element linked to a luciferase gene whose activation reflects and measures ADCC activity. The Kasumi-3 cells are human lymphoblasts expressing the CD123 receptors and were used in this bioassay as target for DART-A. When DART-A is added in the presence of the Jurkat effector cells and Kasumi3 target cells, it binds to the CD123 receptors on the Kasumi cells and the CD3 receptor on the Jurkat Effector cells. This crosslinking leads to the activation of the Jurkat effector cell and the NFAT pathway. The NFAT response element is than able to drive expression of the firefly Luciferase reporter gene which after addition of a detection substrate (Bio-Glo), can accurately and directly be quantified using a luminescence reader.

For this assay, one test article may be run on a single assay plate, along with the DART-A Reference Standard. Additional Test Articles may be run by including multiple plates. The DART-A Reference Standard must be included on each assay plate and is diluted along with the test articles to concentrations ranging from 150 to 0.0006 ng/mL (final concentration 50 to 0.002 ng/mL). Single use thaw and go vials of cells were diluted to specified concentration and are incubated in a 96 well plate with DART-A dilutions for 20-24 hr at 37° C. The ADCC reporter gene activation of the Jurkat effector cells was then assessed using Bio-Glo via the measured amount of luminescence emitted by the enzymatic reaction. Data were fitted to a 4-parameter constrained model and the EC50, the concentration of test article inducing half of the response, was determined and compared to the Reference Standard's EC50. The Reportable Result is the Relative Potency, calculated for each Test Article relative to the DART-A Reference Standard on the same assay plate.

### 7.11. RP-HPLC For BA and MP Recovery

Preservative recovery was measured using Reverse Phase (RP) HPLC analysis. The RP-HPLC configuration included a photodiode array (PDA) detector (λ_{BA}=254 nm, λ_{MP}=340 nm), a mobile phase composition of 40% acetonitrile, 0.1% formic acid and a Zorbax® 5 μm Eclipse-XDB-C18 80 Å liquid chromatography column. Samples were diluted ten-fold. The column temperature was set to 40±5° C. 50 μL of solution was injected for each 10-minute run.

### 7.12. Microbial Challenge Testing

Microbial Challenge Testing was performed to evaluate the preservative effect of stabilizer solutions. In short, high concentration (1250 ng/mL) dosing solution was prepared according Table 36. USP<51> recommended microbes were introduced to the solution with a target concertation between 10 and 100 CFU/mL. Solutions were stored at the USP<51> recommended temperature for each microbe and at predetermined time points, samples were collected. Testing of samples was performed according to USP<1227>. The threshold for no microbial growth was established to be not greater than 0.5 log colony count compared to the initial concentration of microbe.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A stable aqueous pharmaceutical formulation comprising a diabody, a sodium phosphate buffer, sodium chloride, and polysorbate (PS80).

2. The stable aqueous pharmaceutical formulation of claim 1, wherein said sodium phosphate has a concentration of about 5 mM to about 30 mM.

3. The stable aqueous pharmaceutical formulation of claim 2, wherein said concentration of sodium phosphate is about 10 mM.

4. The stable aqueous pharmaceutical formulation of any one of claims 1-3, wherein said PS80 has a concentration of about 0.05 mg/mL to about 0.3 mg/mL.

5. The stable aqueous pharmaceutical formulation of claim 4, where said concentration of PS80 is about 0.1 mg/mL.

6. The stable aqueous pharmaceutical formulation of any one of claims 1-5, wherein said sodium chloride has a concentration of about 100 mM to about 300 mM.

7. The stable aqueous pharmaceutical formulation of claim 6, wherein the concentration of sodium chloride is about 150 mM.

8. The stable aqueous pharmaceutical formulation of any one of claims 1-7, wherein said formulation has a pH of about 5.5 to about 7.0.

9. The stable aqueous pharmaceutical formulation of claim 8, wherein said pH is about 6.0.

10. The stable aqueous pharmaceutical formulation of any one of claims 1-11, wherein said formulation comprises about 10 mM sodium phosphate, about 150 mM sodium chloride, and about 0.1 mg/mL PS80, and wherein said formulation has a pH of about 6.0.

11. The stable aqueous pharmaceutical formulation of any one of claims 1-11, wherein said diabody has a concentration of about 0.01 mg/mL to about 1 mg/mL.

12. The stable aqueous pharmaceutical formulation of any one of claims 1-11, wherein said concentration of diabody is about 0.1 mg/mL.

13. The stable aqueous pharmaceutical formulation of any one of claims 1-12, wherein said diabody is a covalently bonded bispecific diabody having two, three, or four polypeptide chains.

14. The stable aqueous pharmaceutical formulation of any one of claims 1-13, wherein said diabody is a CD123×CD3 diabody.

15. The stable aqueous pharmaceutical formulation of claim 14, wherein said CD123×CD3 diabody comprises:
a) a first polypeptide chain having the amino acid sequence of SEQ ID NO:42; and
b) a second polypeptide chain having the amino acid sequence of SEQ ID NO:44; and
wherein the first and said second polypeptide chains are covalently bonded to one another by a disulfide bond.

16. The stable aqueous pharmaceutical formulation of any one of claims 1-15, wherein said formulation comprises about 0.1 mg/mL of said diabody, about 10 mM sodium phosphate, about 150 mM sodium chloride, and about 0.1 mg/mL PS80, and wherein the pH of said formulation is about 6.0.

17. The stable aqueous pharmaceutical formulation of any one of claims 1-16, wherein said solution maintains monomeric purity of said diabody for about 3 months at 25° C.

18. The stable aqueous pharmaceutical formulation of any one of claims 1-16, wherein said solution maintains monomeric purity of said diabody for about 48 months at 2-8° C.

19. A container comprising the stable aqueous pharmaceutical formulation of any one of claims 1-18.

20. The container of claim 19, wherein said container is a glass vial.

21. A sealed package comprising the stable aqueous pharmaceutical formulation of any one of claims 1-18 or the container of any one of claim 19 or 20.

22. An aqueous stabilizer solution for stabilizing a diabody, comprising sodium phosphate, PS80, benzyl alcohol (BA), and methylparaben (MP).

23. The aqueous stabilizer solution of claim 20, wherein said sodium phosphate has a concentration of about 15 mM to about 25 mM.

24. The aqueous stabilizer solution of claim 21, wherein said concentration of sodium phosphate is about 20 mM.

25. The aqueous stabilizer solution of any one of claims 22-24, wherein said BA has a concentration of about 11.5 mg/mL to about 15.5 mg/mL.

26. The aqueous stabilizer solution of claim 25, wherein said concentration of BA is about 13.2 mg/mL.

27. The aqueous stabilizer solution of any one of claims 22-26, wherein said MP has a concentration of about 3.5 mg/mL to about 5.5 mg/mL.

28. The aqueous stabilizer solution of claim 27, wherein said concentration of MP is about 4.25 mg/mL.

29. The aqueous stabilizer solution of any one of claims 22-28, wherein said PS80 has a concentration of about 0.1 mg/mL to about 0.4 mg/mL.

30. The aqueous stabilizer solution of claim 29, wherein said concentration of PS80 is about 0.25 mg/mL.

31. The aqueous stabilizer solution of any one of claims 22-30, wherein said solution has a pH of about 7.7 to about 8.7.

32. The aqueous stabilizer solution of claim 21, wherein said pH is about 8.2.

33. The aqueous stabilizer solution of any one of claims 22-32, wherein said stabilizer solution comprises about 20 mM sodium phosphate, about 13.2 mg/mL BA, about 4.25 mg/mL MP and about 0.25 mg/mL PS80, and wherein the pH of said solution is about 8.2.

34. The aqueous stabilizer solution of any one of claims 22-33, wherein said diabody is a covalently bonded bispecific diabody having two, three, or four polypeptide chains.

35. The aqueous stabilizer solution of any one of claims 22-34, wherein said diabody is a CD123×CD3 diabody.

36. The aqueous stabilizer solution of claim 35, wherein said CD123×CD3 diabody comprises:
a) a first polypeptide chain having the amino acid sequence of SEQ ID NO:42; and
b) a second polypeptide chain having the amino acid sequence of SEQ ID NO:44; and
wherein the first and said second polypeptide chains are covalently bonded to one another by a disulfide bond.

37. The aqueous stabilizer solution of any one of claims 22-36, wherein said solution maintains monomeric purity of said diabody for about 5-7 days at about 25° C.

38. The aqueous stabilizer solution of any one of claims 22-37, wherein said solution prevents microbial growth for about 5-7 days at about 25° C.

39. A container comprising the aqueous stabilizer solution of any one of claims 22-38.

40. The container of claim 39, wherein said container is a glass vial.

41. A sealed package comprising said aqueous stabilizer solution of any one of claims 23-38 or the container of any one of claim 39 or 40.

42. An aqueous stabilizer solution for stabilizing a diabody, comprising sodium chloride and a PS80.

43. The aqueous stabilizer solution of claim 42, wherein said sodium chloride has a concentration of about 100 mM to about 300 mM.

44. The aqueous stabilizer solution of claim 43, wherein the concentration of said sodium chloride is about 150 mM.

45. The aqueous stabilizer solution of any one of claims 42-44, wherein said PS80 has a concentration of about 0.05 mg/mL to about 0.3 mg/mL.

46. The aqueous stabilizer solution of claim 45, wherein the concentration of said PS80 is about 0.10 mg/mL.

47. The aqueous stabilizer solution of any one of claims 42-46, wherein said solution has a pH of about 5.5 to about 7.0.

48. The aqueous stabilizer solution of claim 47, wherein said pH is 6.0.

49. The aqueous stabilizer solution of any one of claims 42-48, wherein said solution comprise about 150 mM sodium chloride, about 0.1 mg/mL PS80, and wherein the pH of said solution is about 6.0.

50. The aqueous stabilizer solution of any one of claims 42-49, wherein said diabody is a covalently bonded bispecific diabody having two, three, or four polypeptide chains.

51. The aqueous stabilizer solution of any one of claims 42-50, wherein said diabody is a CD123×CD3 diabody.

52. The aqueous stabilizer solution of claim 51, wherein said CD123×CD3 diabody comprises:
a) a first polypeptide chain having the amino acid sequence of SEQ ID NO:42; and
b) a second polypeptide chain having the amino acid sequence of SEQ ID NO:44; and
wherein the first and said second polypeptide chains are covalently bonded to one another by a disulfide bond.

53. The aqueous stabilizer solution of any one of claims 42-52, wherein said solution maintains monomeric purity of said diabody for about 3-5 days at about 25° C.

54. The aqueous stabilizer solution of any one of claims 42-53, wherein said solution prevents microbial growth for about 3-5 days at about 25° C.

55. A container comprising the aqueous stabilizer solution of any one of claims 42-54.

56. The container of claim 55, wherein said container is a glass vial.

57. A sealed package comprising said aqueous stabilizer solution of any one of claims 42-54 or the container of any one of claim 55 or 56.

58. An aqueous stabilizer solution for stabilizing a diabody, comprising one or more of sodium phosphate, sodium chloride, a PS80, and a BA.

59. The aqueous stabilizer solution of claim 58, wherein said sodium phosphate has a concentration of about 5 mM to about 30 mM.

60. The aqueous stabilizer solution of claim 59, wherein the concentration of said sodium phosphate is about 10 mM.

61. The aqueous stabilizer solution of any one of claim 58 or 59, wherein said sodium chloride has a concentration of about 100 mM to about 300 mM.

62. The aqueous stabilizer solution of claim 61, wherein the concentration of said sodium chloride is about 150 mM.

63. The aqueous stabilizer solution of any one of claims 58-62, wherein said BA has a concentration of about 7.0 mg/mL to about 11.0 mg/mL.

64. The aqueous stabilizer solution of claim 63, wherein the concentration of said BA is about 9.0 mg/mL.

65. The aqueous stabilizer solution of any one of claims 58-64, wherein said PS80 has a concentration of about 0.05 mg/mL to about 0.3 mg/mL.

66. The aqueous stabilizer solution of claim 65, wherein the concentration of said PS80 is about 0.10 mg/mL.

67. The aqueous stabilizer solution of any one of claims 58-66, further comprising recombinant human albumin (rHA).

68. The aqueous stabilizer solution of claim 67, wherein said rHA has a concentration of about 0.05 mg/mL to about 0.15 mg/mL.

69. The aqueous stabilizer solution of claim 68, wherein the concentration of said rHA is about 0.10 mg/mL.

70. The aqueous stabilizer solution of any one of claims 58-69, wherein said solution has a pH of about 5.5 to about 7.0.

71. The aqueous stabilizer solution of claim 70, wherein said pH is 6.0.

72. The aqueous stabilizer solution of any one of claims 58-71, wherein said solution comprises:
a) about 10 mM sodium phosphate, about 150 mM sodium chloride, about 9.0 mg/mL BA, about 0.1 mg/mL PS80, and about 0.1 mg/mL rHA, and wherein the pH of said solution is about 6.0; or
b) about 10 mM sodium phosphate, about 150 mM sodium chloride, about 0.1 mg/mL PS80, and wherein the pH of said solution is about 6.0.

73. The aqueous stabilizer solution of any one of claims 58-72, wherein said diabody is a covalently bonded bispecific diabody having two, three, or four polypeptide chains.

74. The aqueous stabilizer solution of any one of claims 58-73, wherein said diabody is a CD123×CD3 diabody.

75. The aqueous stabilizer solution of claim 74, wherein said CD123×CD3 diabody comprises:
a) a first polypeptide chain having the amino acid sequence of SEQ ID NO:42; and
b) a second polypeptide chain having the amino acid sequence of SEQ ID NO:44; and
wherein the first and said second polypeptide chains are covalently bonded to one another by a disulfide bond.

76. The aqueous stabilizer solution of any one of claims 58-75, wherein said solution maintains monomeric purity of said diabody for about 3-5 days at about 25° C.

77. The aqueous stabilizer solution of any one of claims 58-76, wherein said solution prevents microbial growth for about 3-5 days at about 25° C.

78. A container comprising the aqueous stabilizer solution of any one of claims 58-77.

79. The container of claim 78, wherein said container is a glass vial.

80. A sealed package comprising said aqueous stabilizer solution of any one of claims 58-77 or the container of any one of claim 78 or 79.

81. A kit comprising:
a) a container A comprising a stable aqueous pharmaceutical formulation, said formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and said formulation has a pH of about 5.5 to about 7.0; and
b) a container B comprising an aqueous stabilizer solution for stabilizing said diabody, said solution comprising about 15 mM to about 25 mM sodium phosphate buffer, about 11.5 mg/mL to about 15.5 mg/mL BA, about 3.5 mg/mL to about 5.5 mg/mL MP and about 0.1 mg/mL to about 0.4 mg/mL PS80, and said solution has a pH of about 7.7 to about 8.7; and
c) instructions for combining the contents of containers A and B for administration of the combined solution to a subject in need thereof.

82. A kit comprising:
a) a container A comprising a stable aqueous pharmaceutical formulation, said formulation comprising about 0.01 mg/mL to about 1 mg/mL of a diabody, about 5 mM to about 30 mM sodium phosphate buffer, about 100 mM to about 300 mM sodium chloride and about 0.05 mg/mL to about 0.3 mg/mL PS80, and said formulation has a pH of about 5.5 to about 7.0; and
b) (i) a container B comprising an aqueous stabilizer solution for stabilizing said diabody, said solution comprising about 5 mM to about 30 mM sodium phosphate, about 100 mM to about 300 mM sodium chloride, about 7.0 mg/mL to about 11.0 mg/mL BA, about 0.05 mg/mL to about 0.15 mg/mL PS80, and said solution has a pH of about 5.5 to about 7.0; or
(ii) a container B comprising an aqueous stabilizer solution for stabilizing said diabody, said solution comprising about 5 mM to about 30 mM sodium phosphate, about 100 mM to about 300 mM sodium chloride, about 0.05 mg/mL to about 0.15 mg/mL PS80, and said solution has a pH of about 5.5 to about 7.0; or
(iii) a container B comprising an aqueous stabilizer solution for stabilizing said diabody, said solution comprising about 100 mM to about 300 mM sodium chloride, about 0.05 mg/mL to about 0.15 mg/mL PS80, and said solution has a pH of about 5.5 to about 7.0; and optionally,
c) instructions for combining the contents of containers A and B for administration of the combined solution to a subject in need thereof.

83. The kit of any one of claim 81 or 82, wherein said diabody is a covalently bonded bispecific diabody having two, three, or four polypeptide chains.

84. The kit of any one of claims 81-83, wherein said diabody is a CD123×CD3 diabody.

85. The kit of claim 84, wherein said diabody comprises:
a) a first polypeptide chain having the amino acid sequence of SEQ ID NO:42; and
b) a second polypeptide chain having the amino acid sequence of SEQ ID NO:44; and
wherein the first and said second polypeptide chains are covalently bonded to one another by a disulfide bond.

86. The kit of any of claims 81-85, wherein said stable aqueous pharmaceutical formulation in container A comprises about 0.1 mg/mL of said diabody, about 10 mM sodium phosphate, about 150 mM sodium chloride, and about 0.1 mg/mL PS80, and wherein said formulation has a pH of about 6.0.

87. The kit of any one of claim 81 or 83-86, wherein said aqueous stabilizer solution in container B comprises about 20 mM sodium phosphate, about 13.2 mg/mL BA, about 4.25 mg/mL MP and about 0.25 mg/mL PS80.

88. The kit of any one of claims 82-86, wherein said wherein said aqueous stabilizer solution in container B comprises:
Ii) about 10 mM sodium phosphate, about 150 mM sodium chloride, about 9.0 mg/mL BA, about 0.1 mg/mL PS80, and wherein said solution has a pH of about 6.0; or
(ii) about 10 mM sodium phosphate, about 150 mM sodium chloride, about 0.1 mg/mL PS80, and wherein said solution has a pH of about 6.0; or
(iii) about 150 mM sodium chloride, about 0.1 mg/mL PS80, and wherein said solution has a pH of about 6.0.

89. The kit of any one of claims 82-86, wherein said aqueous stabilizer solution further comprises rHA at a concentration of about 0.05 mg/mL to about 0.15 mg/mL.

90. The kit of claim 89, wherein said concentration of rHA is about 0.1 mg/mL.

91. The kit of any one of claims 81-90, wherein said subject is a human patient.

92. The kit of any one of claims 81-91, wherein said container A and container B are glass vials.

93. A sealed package comprising said kit and optionally instructions for storage and/or use of said kit of any one of claims 81-92.

94. A method of administering a diabody to a subject in need thereof comprising using the kit of any one of claim 81, 83-85, 87, or 91-93, wherein said aqueous stabilizer solution of the container B comprises said sodium phosphate, PS80, BA, MP, and has a pH of about 7.7 to about 8.7;
and wherein in the method:
(a) the aqueous stabilizer solution of container B is placed in a container C and mixed;
(b) the stable aqueous pharmaceutical formulation of container A is placed in the container C and mixed to obtain a dosing solution;
(c) the container C containing the dosing solution is attached to a device for administration to the subject.

95. The method of claim 94, wherein said container C comprises saline for intravenous infusion.

96. A method of administering a diabody to a subject in need thereof comprising using the kit of any one of claim 82-86, or 88-93, wherein the aqueous stabilizer solution of the container B comprises one or more of sodium phosphate, sodium chloride, BA, PS80, and optionally rHA, and has a pH of about 5.5 to about 7.0;
and wherein in the method:
(a) the stable aqueous pharmaceutical formulation of the container A is placed in the aqueous stabilizer solution of the container B and mixed to obtain a dosing solution;
(b) optionally diluting the dosing solution;
(c) placing the dosing solution in a container C; and
(d) attaching the container C containing the final dosing solution to a device for administration to the subject.

97. The method of claim 96, wherein said container C comprises saline or bacteriostatic saline for intravenous infusion.

98. The method of any one of claims 94-97, wherein said administration is by infusion pump.

99. The method of any one of claims 94-98, wherein said administration is ambulatory.

100. The method of any one of claims 94-98, wherein said device is a single ambulatory pump.

101. The method of any one of claims 94-98, wherein said device is a dual ambulatory pump.

102. The method of any one of claims 94-98, wherein said device is a syringe pump.

103. The method of any one of claims 94-102, wherein said administration is by continuous infusion for at least about 24 hours.

104. The method of any one of claims 94-102, wherein said administration is by continuous infusion for at least about 48 hours.

105. The method of any one of claims 94-102, wherein said administration is by continuous infusion for at least about 96 hours.

106. The method of any one of claims 94-102, wherein said administration is by continuous infusion for at least about 7 days.

107. The method of any one of claims 94-106, wherein said administration occurs at a flow rate of about 0.10 mL/hour to about 2.5 mL/hour.

108. The method of any one of claims 94-106, wherein said administration occurs at a flow rate of about 0.5 mL/hour to about 10.0 mL/hour.

109. The method of any one of claims 94-102, wherein said administration is by continuous infusion for at least 24 hours at a flow rate of about 0.1 mL/hour to about 2.0 mL/hour.

110. The method of any one of claims 94-102, wherein said administration is by continuous infusion for at least 48 hours at a flow rate of about 0.5 mL/hour to about 6 mL/hour.

111. The method of any one of claims 94-102, wherein said administration is by continuous infusion for at least 96 hours at a flow rate of about 0.6 mL/hour to about 3.0 mL/hour.

112. The method of any one of claims 94-102, wherein said administration is by continuous infusion for at least 96 hours at a flow rate of about 0.3 mL/hour to about 3.0 mL/hour.

113. The method of any one of claim 94-102 or 112, wherein said administration is by continuous infusion for at least 96 hours at a flow rate of about 0.5 mL/hour.

114. The method of any one of claims 94-102, wherein said administration is by continuous infusion for at least 7 days at a flow rate of about 0.3 mL/hour to about 3.0 mL/hour.

115. The method of any one of claim 94-102 or 114, wherein said administration is by continuous infusion for at least 7 days at a flow rate of about 0.5 mL/hour.

116. The method any one of claims 94-115, wherein said flow rate prevents vein occlusion in said subject.

117. The method of any one of claims 94-116, wherein said diabody is administered to said subject at a treatment dosage selected from the group consisting of 30-500 ng/kg/day.

118. The method of any one of claim 94-95, 97-99, or 102-117 wherein said dosing solution comprises 40 mL of aqueous stabilizer solution.

119. The method of any one of claim 94-95, 97-99, or 102-118, wherein said dosing solution comprises about 0.03 mg/mL to about 0.04 mg/mL PS80, about 1.7 mg/mL to about 2.1 mg/mL BA, and about 0.55 mg/mL to about 0.7 mg/mL MP.

120. The method of any one of claims 96-118, wherein said dosing solution comprises about 100 mM to about 300 mM sodium chloride, about 0.05 mg/mL to about 0.15 mg/mL PS80, and said solution has a pH of about 5.5 to about 7.0.

121. The method of any one of claims 94-120, wherein said patient is a human subject.

122. A method of treating a hematologic malignancy comprising administering a diabody to a subject in need thereof, according to the method of administering of any one of claims 94-121 or using the kit of any one of claims 81-92.

123. The use of the kit of any one of claims 81-92 for the treatment of a hematologic malignancy.

124. The method of claim 122, or the use of claim 123, wherein said hematologic malignancy is selected from the group consisting of: acute myeloid leukemia (AML, chronic myelogenous leukemia (CIVIL, including blastic crisis of CIVIL and Abelson oncogene associated with CML (Bcr-ABL translocation, myelodysplastic syndrome (MDS, acute B lymphoblastic leukemia (B-ALL, acute T lymphoblastic leukemia (T-ALL, chronic lymphocytic leukemia (CLL, including Richter's syndrome or Richter's transformation of CLL, hairy cell leukemia (HCL, blastic plasmacytoid dendritic cell neoplasm (BPDCN, non-Hodgkin's lymphoma (NHL, including mantle cell lymphoma (MCL) and small lymphocytic lymphoma (SLL, Hodgkin's lymphoma, systemic mastocytosis, and Burkitt's lymphoma.

125. The method or the use of claim 124, wherein said hematologic malignancy is AML.

126. The method or the use of claim 124, wherein said hematologic malignancy is BPDCN.

127. The method or the use of claim 124, wherein said hematologic malignancy is MDS.

128. The method or the use of claim 124, wherein said hematologic malignancy is T-ALL.

129. The method or the use of any one of claims 122-128, wherein said subject is a human subject.
